(19) United States
(12) Patent Application Publication
(10) Pub. No.: US 2026/0234223 A1
(43) Pub. Date: Aug. 13, 2026

(54) Method For Prevention or Treatment of Coronavirus Infection

(71) Applicant: Leyden Laboratories B.V., Leiden (NL)

(72) Inventors: Jaap Goudsmit, Amsterdam (NL); Michael Alan Shaw, Leiden (NL)

(21) Appl. No.: 19/150,738

(22) PCT Filed: Jan. 25, 2024
(86) PCT No.: PCT/EP2024/051834

§ 371 Date: Jul. 24, 2025

(30) Foreign Application Priority Data
Jan. 25, 2023 (EP) 23153354.8
Aug. 9, 2023 (EP) 23190707.2

(51) Int. Cl. C07K 16/104; A61K 9/00; A61K 39/00; A61P 31/14

(52) U.S. Cl. CPC C07K 16/104; A61K 9/0043; A61P 31/14; A61K 2039/505; A61K 2039/543

(57) ABSTRACT

The invention is in the field of medical treatment and relates to a method for treating coronavirus infection. In particular, the present invention relates to methods for prophylactic and/or therapeutic treatment of coronavirus infection by means of the mucosal administration of antibodies against coronavirus.

### CROSS REFERENCE TO RELATED APPLICATIONS

This application is a national phase entry under 35 U.S.C. § 371 of International Patent Application PCT/EP2024/051834, filed Jan. 25, 2024, designating the United States of America and published in English as International Patent Publication WO2024/156832 on Aug. 2, 2024, which claims the benefit under Article 8 of the Patent Cooperation Treaty to European Patent Application Serial No. 23153354.8, filed Jan. 25, 2023, and to European Patent Application Serial No. 23190707.2, filed Aug. 9, 2023, the entireties of which are hereby incorporated by reference.

### INCORPORATION BY REFERENCE

The ST.26 XML Sequence listing named “11018-US Sequence Listing ST26”, created on Mar. 11, 2024, and having a size of 577,536 bytes, is hereby incorporated herein by this reference in its entirety.

### FIELD OF THE INVENTION

The invention is in the field of medical treatment and relates to a method for treating β-coronavirus infections in animals and humans, including Severe Acute Respiratory Syndrome Coronavirus 2 (SARS-CoV-2).

In particular, the present invention relates to methods for prophylactic and/or therapeutic treatment of SARS-CoV-2 by means of mucosal administration, especially intranasal and/or oral inhalation of antibodies against β-coronavirus, in particular SARS-CoV-2.

### BACKGROUND OF THE INVENTION

The SARS-CoV-2 virus causes the disease COVID-19 in humans and is widespread having a significant impact on human society. We should expect the SARS-CoV-2 virus and its variants of concern to continue to cause frequent infections, as well as severe disease and death. As of January 2023, the ongoing COVID-19 pandemic caused by SARS-CoV-2, has resulted in more than 6.7 million worldwide deaths since 2019.

In recent years, outbreaks of other coronaviruses have caused severe illness and deaths. As scientists develop therapeutic antibodies and vaccines against SARS-CoV-2, the danger remains of future novel variants of SARS-CoV-2 and novel coronaviruses emerging. These novel coronaviruses can lead to morbidity, mortality, and could cause pandemics. It remains vitally important to identify broadly protective therapies that can combat current and emerging coronaviruses in the future.

In addition to SARS-CoV-2, six other coronaviruses are known to cause disease in humans: the betacoronaviruses HCoV-OC43 (human coronavirus OC43), HCoV-HKU1 (human coronavirus HKU1), SARS-CoV (severe acute respiratory syndrome coronavirus) and MERS-COV (Middle East respiratory syndrome coronavirus). Spillover events, where humans get infected with coronaviruses that circulate in animal reservoirs, are common. Recently, three betacoronaviruses crossed from animals to humans, including SARS-CoV-2 causing serious outbreaks.

Furthermore, two coronaviruses previously linked only to animal infection were recently detected in humans who presented with flu-like symptoms.

Coronaviruses derive their name from their crown-like appearance. Coronaviruses are a large group of viruses that have spike proteins on their surface, resembling crown-like thorns. The antibodies elicited by natural infection or by COVID-19 vaccines used for mass immunization between 2020-2023 primarily target the variable receptor binding domain (RBD) present on these spike proteins.

### Structure of the Spike Protein

Coronavirus infection is a multistep process that involves enzymatic cleavage and rearrangement of the surface spike protein. The spike protein has an S1 subunit with the receptor-binding domain and an S2 subunit involved in fusion of the viral and cell membranes thereby facilitating cell entry.

The viral spike protein of SARS-CoV-2 facilitates viral entry by binding primarily to the angiotensin-converting enzyme 2 (ACE2) receptor on human cells.

The SARS-CoV-2 spike contains two cleavage sites: a furin cleavage site at the boundary of the S1 and S2 subunits, and an S2′ site that is highly conserved among different coronaviruses.

### S1 Subunit

The SARS-CoV-2 spike protein uses the RBD on the S1 subunit to engage the target cell's ACE2 receptor.

The S1 subunit is more accessible and remains the main target of many neutralizing antibodies. The S1 subunit, however, is more genetically variable than the S2 subunit; especially when subjected to the selective pressure from antibodies. This propensity towards genetic variability can lead to viral variants with the predominant changes occurring on the S1 subunit. Changes in the receptor-binding domain in SARS-CoV-2 variants of concern have resulted in frequent reinfections, as protection by the antibodies elicited by previous infection and/or vaccines dramatically reduced.

### S2 Subunit

The viral spike components essential to infection also involve the structurally complex S2 subunit. The S2 subunit contains dynamic elements essential for fusion with the host cell. Once the receptor has bound, the S1 subunit is discarded and the membrane enzyme transmembrane serine protease 2 (TMPRSS2) or endosomal cathepsins cleave the S2 site.

This cleaving leads to insertion of the fusion peptide into the cell membrane culminating in viral fusion.

These S2 subunit elements are less disposed towards genetic variability than the RBD, which so far has been capable of retaining or even increasing binding capabilities to ACE2 despite a variety of mutations.

The S2 domain sites yield poorly accessible targets for novel therapeutics to protect against a wider range of coronaviruses.

### The Stem Helix

The stem helix at the base of the viral spike protein is even more difficult to access than elements on the S2 subunit but historically benefits from better-preserved amino acid sequencing.

### Treatments and Prevention

Limited options for the viral treatment of SARS-CoV-2 exist in the form of small molecule or antibody antiviral drugs. For prevention of SARS-CoV-2 infection, few interventions exist. Most are non-pharmaceutical interventions such as social distancing and mask wearing. These methods are non-specific, and efficacy largely depends on compliance. Thus, there is a need for different treatments, especially treatments that simultaneously target the multitude of SARS-CoV-2 variants of concern such as, for example, broad-neutralizing antibodies (bnAbs).

Although treatment of SARS-CoV-2 infection with one, two or more antibodies is a possibility, a treatment comprising a single antibody or combination of antibodies would be considerably more cost-efficient. The currently dominant SARS-CoV-2 Omicron subvariant BA.5 is resistant to most monoclonal antibody therapeutics. Betacoronaviruses, including SARS-CoV-2, also infect a range of species know to have frequent human contact, thus increasing the risk of zoonotic transfer and new outbreaks with SARS-CoV-2 variants or viruses that previously have not infected humans and for which no therapies exist.

There is therefore an urgent need to develop therapeutic mAbs that broadly target β-coronaviruses, as well as new SARS-CoV-2 variants of concern that are not neutralized by antibodies elicited by immunization and/or natural infection.

It remains vitally important to identify broadly protective therapies that can combat novel coronaviruses. We have surprisingly developed a novel array of broadly protective antibodies which are disclosed herein.

### SUMMARY OF THE INVENTION

The inventors have discovered that an antibody having complementarity-determining regions (CDR) as disclosed herein can advantageously be used in the treatment of a coronavirus infection, especially in the prophylactic treatment of coronavirus infection.

The inventors have surprisingly established when treating a coronavirus infection, mucosal administration of an antibody having CDRs as disclosed herein is a more potent prophylactic route of administration method compared with intraperitoneal administration.

The inventors have surprisingly established when treating a coronavirus infection, intranasal administration of an antibody having CDRs as disclosed herein is a more potent prophylactic route of administration method compared with intraperitoneal administration.

Significantly, the mucosal administration method of treatment of the present invention is effective against SARS-Cov-2 and variants of concern and does not require the use of additional antibodies.

A mucosal administration treatment method of the invention is effective even at low dosages and counteracts body weight loss.

### Complementarity-Determining Regions (CDRs)

Preferably, CDR regions are according to Kabat et al., (1991) as described in Sequences of Proteins of Immunological Interest.

In a preferred embodiment, the invention provides an antibody that comprises a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628.

In a preferred embodiment, the invention provides an antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628, wherein the antibody is administered to mucosal epithelium.

In a preferred embodiment, the invention provides a method for the treatment of a coronavirus infection in an individual, the method comprising administering to an individual in need thereof an antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628, wherein the antibody is administered to mucosal epithelium.

In a preferred embodiment, the invention provides a mucosal composition comprising an antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628.

In a preferred embodiment, the invention provides an antibody for use in a method for treatment of a coronavirus infection in an individual, wherein the antibody comprises a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628, wherein the antibody is administered to mucosal epithelium.

In a preferred embodiment, the invention provides an antibody, or a method or composition comprising the antibody, wherein the antibody comprises a heavy chain variable domain that comprises a heavy chain CDR1 region comprising SEQ ID NO: 029, a heavy chain CDR2 region comprising SEQ ID NO: 136, and a heavy chain CDR3 region comprising SEQ ID NO: 256, a light chain variable domain that comprises a light chain CDR1 region comprising SEQ ID NO: 372, a light chain CDR2 region comprising SEQ ID NO: 432, and a light chain CDR3 region comprising SEQ ID NO: 573.

In a preferred embodiment, the invention provides an antibody, or a method or composition comprising the antibody, wherein the antibody comprises a heavy chain variable domain that comprises a heavy chain CDR1 region consisting of SEQ ID NO: 029, a heavy chain CDR2 region consisting of SEQ ID NO: 136, and a heavy chain CDR3 region consisting of SEQ ID NO: 256, a light chain variable domain that comprises a light chain CDR1 region consisting of SEQ ID NO: 372, a light chain CDR2 region consisting of SEQ ID NO: 432, and a light chain CDR3 region consisting of SEQ ID NO: 573.

In a preferred embodiment, an antibody is disclosed herein for use in a method for the prophylactic treatment of a coronavirus infection, in an individual, the antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628, and wherein between 0.1 mg and 20 mg of the antibody is administered to the mucosa.

In a preferred embodiment, an antibody is disclosed herein for use in a method for the prophylactic treatment of a coronavirus infection, in an individual, the antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628, and wherein between 0.1 mg and 20 mg of the antibody is administered intranasally.

In a preferred embodiment, an antibody is disclosed herein for use in a method for the prophylactic treatment of a coronavirus infection, in an individual, the antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628, and wherein between 0.1 mg and 20 mg of the antibody is administered by oral inhalation.

In a preferred embodiment, a composition comprising an antibody is disclosed herein, the antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628, for use in a method of prevention or treatment of a coronavirus infection, wherein the administration route comprises at least one of pulmonary administration, nasal administration and oropharyngeal administration and wherein the nominal dose of the antibody is between 0.1 mg and 20 mg.

Preferably, an antibody as disclosed herein comprises a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302.

Preferably, an antibody as disclosed herein comprises a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628.

Preferably, in a method for treatment of the invention, the antibody comprises a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302.

Preferably, in a method for treatment of the invention, the antibody comprises a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628.

In an alternative preferred embodiment, the invention provides an antibody that comprises a heavy chain variable domain that comprises a heavy chain CDR1 region comprising any one of SEQ ID NO: 004 to 009, 015 to 020, 030 to 034, 039 to 044, 047 to 052, 050 to 054, a heavy chain CDR2 region comprising any one of SEQ ID NO: 082 to 087, 103 to 108, 125 to 130, 137 to 141, 147 to 152, 132 to 136, and a heavy chain CDR3 region comprising any one of SEQ ID NO: 210 to 215, 221 to 226, 245 to 250, 257 to 261, 286 to 291, 252 to 256, a light chain variable domain that comprises a light chain CDR1 region comprising any one of SEQ ID NO: 319 to 324, 330 to 335, 350 to 355, 373 to 377, 383 to 388, 358 to 362, a light chain CDR2 region comprising any one of SEQ ID NO: 433 to 437, 443 to 448, 454 to 459, 465 to 470, 476 to 481, 475 to 479, and a light chain CDR3 region comprising any one of SEQ ID NO: 529 to 534, 540 to 545, 574 to 578, 601 to 606, 612 to 617, 569 to 573.

### Embodiments Having >1 CDRs

Preferably, an antibody as disclosed herein comprises a heavy chain variable domain that comprises a heavy chain CDR2 region comprising any one of SEQ ID NO: 082 to 087, 103 to 108, 125 to 130, 136 to 141, 147 to 152, 191 to 196.

### Route of Administration

In a preferred embodiment, the invention provides an antibody as disclosed herein, or a method or composition comprising the antibody as disclosed herein, wherein the antibody is administered by mucosal administration.

In a preferred embodiment, the invention provides an antibody as disclosed herein, or a method or composition comprising the antibody as disclosed herein, wherein the antibody is administered by intravenous administration.

In a preferred embodiment, the invention provides an antibody as disclosed herein, or a method or composition comprising the antibody as disclosed herein, wherein the antibody is administered by at least one of oral inhalation, nasal administration, ocular administration, vaginal administration, rectal administration and oropharyngeal administration.

In a preferred embodiment, the antibody as disclosed herein is administered intranasally.

Such an antibody is useful for the treatment of coronavirus infection in an individual. Preferably, the method for treatment of coronavirus infection is a method for prophylactic and/or therapeutic treatment of a coronavirus infection. Preferably, the antibody is provided to an individual infected with coronavirus. Preferably, the antibody is provided to the individual prophylactically. Preferably, the antibody is provided to the individual after infection but prior to onset of symptoms.

In a preferred embodiment, the disclosure provides a method of treating coronavirus infection in an individual, said method comprising mucosal administration, preferably administering intranasally and/or by oral inhalation, to an individual in need thereof an antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628.

In a preferred embodiment, the individual is infected with coronavirus or at risk of coronavirus infection.

In a preferred embodiment, the disclosure provides an antibody as disclosed herein for use in the manufacture of a medicament for use in the treatment of a coronavirus infection.

### CLAUSES

The following numbered clauses each represent preferred embodiments of the invention and are part of the description.

### Clause 1

A method for the treatment of a coronavirus infection in an individual, the method comprising administering to an individual in need thereof an antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628, wherein the antibody is administered to the mucosa.

### Clause 2

A mucosal composition comprising an antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628.

### Clause 3

An antibody for use in a method of prevention or treatment of a coronavirus infection in an individual, wherein the antibody comprises a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628, wherein the antibody is administered to mucosa.

### Clause 4

A method, composition or antibody according to any preceding Clause, wherein the antibody comprises a heavy chain variable domain that comprises a heavy chain CDR1 region comprising SEQ ID NO: 029, a heavy chain CDR2 region comprising SEQ ID NO: 136, and a heavy chain CDR3 region comprising SEQ ID NO: 256, a light chain variable domain that comprises a light chain CDR1 region comprising SEQ ID NO: 372, a light chain CDR2 region comprising SEQ ID NO: 432, and a light chain CDR3 region comprising SEQ ID NO: 573.

### Clause 5

A method, composition or antibody according to any preceding Clause, wherein the method for treatment of coronavirus virus infection is a method for prophylactic and/or therapeutic treatment of a coronavirus, preferably wherein the method for treatment is for the prophylactic treatment of a SARS-CoV-2 infection.

### Clause 6

A method, composition or antibody according to any preceding Clause, wherein the antibody comprises a heavy chain variable domain having the sequence of SEQ ID NO:629 having at most 15, preferably 0, 1, 2, 3, 4, 5, 6, 7, 8, 9 or 10 amino acid insertions, deletions, substitutions which are not in the heavy chain CDRs.

### Clause 7

A method, composition or antibody according to any preceding Clause, wherein the antibody comprises a light chain variable domain having the sequence of SEQ ID NO:630 having at most 15, preferably 0, 1, 2, 3, 4, 5, 6, 7, 8, 9 or 10 amino acid insertions, deletions, substitutions which are not in the light chain CDRs.

### Clause 8

A method, composition or antibody according to any preceding Clause, wherein the antibody is an IgG antibody, preferably an IgG1 antibody.

### Clause 9

A method, composition or antibody according to any preceding Clause, wherein the antibody is provided to the individual prophylactically.

### Clause 10

A method, composition or antibody according to any preceding Clause, wherein the administration route comprises at least one of oral inhalation, nasal administration, ocular administration and oropharyngeal administration.

### Clause 11

A method, composition or antibody according to any preceding Clause, wherein the antibody is administered at least once or at least twice monthly.

### Clause 12

A method, composition or antibody according to any preceding Clause, wherein the antibody is administered to the individual at a dosage of between 0.01 mg and 20 mg.

### Clause 13

A composition according to any preceding Clause, comprising an antibody in a single dose unit of between 0.01 mg and 20 mg, preferably between 0.1 mg and 15 mg or preferably 0.5 mg and 10 mg, wherein the antibody is as defined in any one of Clauses 1 to 12.

### Clause 14

A composition according to any preceding Clause, wherein the composition is self-administered.

### Clause 15

A medicament delivery device comprising a composition according to any preceding Clause.

### Dosing

Preferably, between 0.01 mg and 20 mg of the antibody is administered to an individual, more preferably between 0.1 mg and 20 mg of the antibody is administered to an individual.

The flat dose, or nominal dose, of the antibody is preferably between 0.01 mg and 17 mg, preferably between 0.011 mg and 16 mg, preferably between 0.012 mg and 15 mg, preferably between 0.013 mg and 14 mg, preferably between 0.014 mg and 13 mg, preferably between 0.015 mg and 12 mg, preferably between 0.016 mg and 11 mg, preferably between 0.017 mg and 10 mg, preferably between 0.018 mg and 9 mg, preferably between 0.020 mg and 8 mg, preferably between 0.023 mg and 7 mg, preferably between 0.025 mg and 6 mg, preferably between 0.030 mg and 5 mg, preferably between 0.040 mg and 4 mg, preferably between 0.050 mg and 3 mg, preferably between 0.075 mg and 2 mg, or preferably between 0.10 mg and 1 mg.

The flat dose, or nominal dose, of the antibody is preferably between 0.010 mg and 5.0 mg, preferably between 0.020 mg and 4.5 mg, preferably between 0.030 mg and 4.0 mg, preferably between 0.040 mg and 3.5 mg, preferably between 0.050 mg and 3.0 mg, preferably between 0.060 mg and 2.5 mg, preferably between 0.070 mg and 2.0 mg, preferably between 0.080 mg and 1.5 mg, preferably between 0.090 mg and 1.0 mg, or preferably between 0.100 mg and 0.5 mg.

The flat dose, or nominal dose, of the antibody is preferably between 0.010 mg and 15.0 mg, preferably between 0.020 mg and 14.5 mg, preferably between 0.030 mg and 4.0 mg, preferably between 0.040 mg and 13.5 mg, preferably between 0.050 mg and 13.0 mg, preferably between 0.060 mg and 12.5 mg, preferably between 0.070 mg and 12.0 mg, preferably between 0.080 mg and 11.5 mg, preferably between 0.090 mg and 11.0 mg, or preferably between 0.100 mg and 10.5 mg.

The flat dose, or nominal dose, of the antibody to be delivered to a human subject is preferably between 0.1 μg and 10 μg, preferably between 0.2 μg and 9 μg, preferably between 0.3 μg and 8 μg, preferably between 0.4 μg and 7 μg, preferably between 0.5 μg and 6 μg, preferably between 0.6 μg and 5 μg, preferably between 0.7 μg and 4 μg, preferably between 0.8 μg and 3 μg, preferably between 0.9 μg and 2 μg or preferably around 1 μg.

The flat dose, or nominal dose, of the antibody to be delivered to a human subject is preferably between 1 mg and 11 mg, preferably between 2 mg and 12 mg, preferably between 3 mg and 13 mg, preferably between 4 mg and 14 mg, preferably between 5 mg and 15 mg, preferably between 6 mg and 16 mg, preferably between 7 mg and 17 mg, preferably between 8 mg and 18 mg, preferably between 9 mg and 19 mg, or preferably between 10 mg and 20 mg.

The flat dose, or nominal dose, of the antibody to be delivered to a human subject is preferably between 1 mg and 20 mg, preferably between 2 mg and 19 mg, preferably between 3 mg and 18 mg, preferably between 4 mg and 17 mg, preferably between 5 mg and 16 mg, preferably between 6 mg and 15 mg, preferably between 7 mg and 14 mg, preferably between 8 mg and 13 mg, preferably between 9 mg and 12 mg, or preferably between 10 mg and 11 mg.

The flat dose, or nominal dose, of the antibody to be delivered to a human subject is preferably between 11 mg and 20 mg, preferably between 10 mg and 19 mg, preferably between 9 mg and 18 mg, preferably between 8 mg and 17 mg, preferably between 7 mg and 16 mg, preferably between 6 mg and 15 mg, preferably between 5 mg and 14 mg, preferably between 4 mg and 13 mg, preferably between 3 mg and 12 mg, preferably between 2 mg and 11 mg, or preferably between 1 mg and 10 mg.

In a preferred embodiment, the disclosure further provides a composition formulated for mucosal administration comprising an antibody as disclosed herein in a single dose unit of between 0.1 mg and 20 mg, preferably between 5 mg and 15 mg or preferably 7.5 mg and 12.5 mg.

In a preferred embodiment, the disclosure further provides a composition formulated for intranasal administration comprising an antibody as disclosed herein in a single dose unit of between 0.1 mg and 20 mg, preferably between 5 mg and 15 mg or preferably 7.5 mg and 12.5 mg.

In a preferred embodiment, the disclosure further provides a composition formulated for oral inhalation comprising an antibody as disclosed herein in a single dose unit of between 0.1 mg and 20 mg, preferably between 5 mg and 15 mg or preferably 7.5 mg and 12.5 mg.

### Timing and Intervals

Preferably, the antibody is administered at least once or at least twice per month.

Preferably, the antibody is administered at least once or at least twice per week.

The weekly flat dose, or nominal dose, of the antibody is between 0.010 mg and 17 mg, preferably between 0.011 mg and 16 mg, preferably between 0.012 mg and 15 mg, preferably between 0.013 mg and 14 mg, preferably between 0.014 mg and 13 mg, preferably between 0.015 mg and 12 mg, preferably between 0.016 mg and 11 mg, preferably between 0.017 mg and 10 mg, preferably between 0.018 mg and 9 mg, preferably between 0.020 mg and 8 mg, preferably between 0.023 mg and 7 mg, preferably between 0.025 mg and 6 mg, preferably between 0.030 mg and 5 mg, preferably between 0.040 mg and 4 mg, preferably between 0.050 mg and 3 mg, preferably between 0.075 mg and 2 mg, or preferably between 0.10 mg and 1 mg.

The weekly flat dose, or nominal dose, of the antibody is between 0.010 mg and 5.0 mg, preferably between 0.020 mg and 4.5 mg, preferably between 0.030 mg and 4.0 mg, preferably between 0.040 mg and 3.5 mg, preferably between 0.050 mg and 3.0 mg, preferably between 0.060 mg and 2.5 mg, preferably between 0.070 mg and 2.0 mg, preferably between 0.080 mg and 1.5 mg, preferably between 0.090 mg and 1.0 mg, or preferably between 0.100 mg and 0.5 mg.

Preferably, the antibody is administered at least once or at least twice per day. Preferably, the antibody is administered daily. Preferably, the antibody is administered twice daily.

Preferably, the daily flat dose, or nominal dose, is between 0.001 mg and 17 mg, preferably between 0.011 mg and 16 mg, preferably between 0.012 mg and 15 mg, preferably between 0.013 mg and 14 mg, preferably between 0.014 mg and 13 mg, preferably between 0.015 mg and 12 mg, preferably between 0.016 mg and 11 mg, preferably between 0.017 mg and mg, preferably between 0.018 mg and 9 mg, preferably between 0.020 mg and 8 mg, preferably between 0.023 mg and 7 mg, preferably between 0.025 mg and 6 mg, preferably between 0.030 mg and 5 mg, preferably between 0.040 mg and 4 mg, preferably between 0.050 mg and 3 mg, preferably between 0.075 mg and 2 mg, or preferably between 0.10 mg and 1 mg.

### Framework Regions

Preferably, the antibody comprises a heavy chain variable domain having the sequence of SEQ ID NO: 629 and/or a light chain variable domain having the sequence of SEQ ID NO: 630.

Preferably, the heavy chain variable domain of said antibody further comprises a heavy chain framework region FR1 of SEQ ID NO: 631, a heavy chain framework region FR2 of SEQ ID NO: 632, a heavy chain framework region FR3 of SEQ ID NO: 633, and/or a heavy chain framework region FR4 of SEQ ID NO: 634.

Preferably, said light chain variable domain further comprises a light chain framework region FR1 of SEQ ID NO: 635, a light chain framework region FR2 of SEQ ID NO: 636, a light chain framework region FR3 of SEQ ID NO: 637, and/or a light chain framework region FR4 of SEQ ID NO: 638.

### Combination Compositions

In a preferred embodiment, we disclose a composition comprising a First binding fragment as disclosed herein and a Second binding fragment, wherein the First fragment comprises a sequence comprising any one of SEQ ID NO: 004 to 009, 015 to 020, 029 to 034, 039 to 044, 047 to 052, 065 to 070, 082 to 087, 103 to 108, 125 to 130, 136 to 141, 147 to 152, 191 to 196, 210 to 215, 221 to 226, 245 to 250, 256 to 261, 286 to 291, 297 to 302, 319 to 324, 330 to 335, 350 to 355, 372 to 377, 383 to 388, 415 to 420, 432 to 437, 443 to 448, 454 to 459, 465 to 470, 476 to 481, 508 to 513, 529 to 534, 540 to 545, 573 to 578, 601 to 606, 612 to 617, 623 to 628. More preferably wherein the First fragment comprises a sequence comprising any one or more of SEQ ID NO: 029, 136, 256, 372, 432 or 573. The Second binding fragment comprises a sequence comprising any one of SEQ ID NO.: 639, 640, 641, 642, 643 or SEQ ID NO.: 644. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose a composition comprising a First binding fragment as disclosed herein and a Second binding fragment, wherein the First fragment comprises a sequence comprising any one of SEQ ID NO: 004 to 009, 015 to 020, 029 to 034, 039 to 044, 047 to 052, 065 to 070, 082 to 087, 103 to 108, 125 to 130, 136 to 141, 147 to 152, 191 to 196, 210 to 215, 221 to 226, 245 to 250, 256 to 261, 286 to 291, 297 to 302, 319 to 324, 330 to 335, 350 to 355, 372 to 377, 383 to 388, 415 to 420, 432 to 437, 443 to 448, 454 to 459, 465 to 470, 476 to 481, 508 to 513, 529 to 534, 540 to 545, 573 to 578, 601 to 606, 612 to 617, 623 to 628. More preferably wherein the First fragment comprises any one or more of SEQ ID NO: 029, 136, 256, 372, 432 or 573. The Second binding fragment comprises a sequence comprising any one of SEQ ID NO.: 645, 646, 647, 648, 649 or SEQ ID NO.: 650. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose a composition comprising a First binding fragment as disclosed herein and a Second binding fragment, wherein the First fragment comprises a sequence comprising any one of SEQ ID NO: 004 to 009, 015 to 020, 029 to 034, 039 to 044, 047 to 052, 065 to 070, 082 to 087, 103 to 108, 125 to 130, 136 to 141, 147 to 152, 191 to 196, 210 to 215, 221 to 226, 245 to 250, 256 to 261, 286 to 291, 297 to 302, 319 to 324, 330 to 335, 350 to 355, 372 to 377, 383 to 388, 415 to 420, 432 to 437, 443 to 448, 454 to 459, 465 to 470, 476 to 481, 508 to 513, 529 to 534, 540 to 545, 573 to 578, 601 to 606, 612 to 617, 623 to 628. More preferably wherein the First fragment comprises a sequence comprising any one or more of SEQ ID NO: 029, 136, 256, 372, 432 or 573. The Second binding fragment comprises a sequence comprising any one of SEQ ID NO.: 651, 652, 653, 654, 655 or SEQ ID NO.: 656. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose a composition comprising a First binding fragment as disclosed herein and a Second binding fragment, wherein the First fragment comprises a sequence comprising any one of SEQ ID NO: 004 to 009, 015 to 020, 029 to 034, 039 to 044, 047 to 052, 065 to 070, 082 to 087, 103 to 108, 125 to 130, 136 to 141, 147 to 152, 191 to 196, 210 to 215, 221 to 226, 245 to 250, 256 to 261, 286 to 291, 297 to 302, 319 to 324, 330 to 335, 350 to 355, 372 to 377, 383 to 388, 415 to 420, 432 to 437, 443 to 448, 454 to 459, 465 to 470, 476 to 481, 508 to 513, 529 to 534, 540 to 545, 573 to 578, 601 to 606, 612 to 617, 623 to 628. More preferably wherein the First fragment comprises a sequence comprising any one or more of SEQ ID NO: 029, 136, 256, 372, 432 and 573. The Second binding fragment comprises a sequence comprising any one of SEQ ID NO.: 657, 658, 659, 660, 661 or SEQ ID NO.: 662. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

### Bispecific Antibodies

In a preferred embodiment, we disclose a bispecific antibody, wherein the bispecific antibody is capable of binding to the stem helix of a coronavirus. In a preferred embodiment, we disclose a bispecific antibody, wherein the bispecific antibody is capable of binding to the stem helix of SARS-CoV-2.

In a preferred embodiment, we disclose an anti-coronavirus bispecific antibody or an antigen binding fragment thereof, having a first Fab capable of binding to the stem helix of a coronavirus and a second Fab capable of binding to the fusion peptide of a coronavirus. In a preferred embodiment, we disclose an anti-SARS-CoV-2 bispecific antibody or an antigen binding fragment thereof, having a first Fab capable of binding to the stem helix of SARS-CoV-2 and a second Fab capable of binding to the fusion peptide of SARS-CoV-2.

In a preferred embodiment, we disclose an anti-SARS-CoV-2 bispecific antibody or an antigen binding fragment thereof, having a first Fab capable of binding to the stem helix of SARS-CoV-2 and a second Fab capable of binding to the fusion peptide of SARS-CoV-2, wherein the first Fab comprises a sequence comprising any one or more of preferred SEQ ID Numbers: 029, 136, 256, 372, 432 or 573; alternatively wherein the first Fab comprises a sequence comprising any one or more of preferred SEQ ID Numbers: 651, 652, 653, 654, 655 or 656; or alternatively wherein the first Fab comprises a sequence comprising any one or more of preferred SEQ ID Numbers: 657, 658, 659, 660, 661 to 662, and wherein the second Fab comprises a sequence comprising any one or more of preferred SEQ ID Numbers: 639, 640, 641, 642, 643 to 644; or alternatively wherein the second Fab comprises a sequence comprising any one or more of preferred SEQ ID Numbers: 645, 646, 647, 648, 649 to 650.

In a preferred embodiment, we disclose an anti-SARS-CoV-2 bispecific antibody or an antigen binding fragment thereof, having a first Fab capable of binding to the stem helix of SARS-CoV-2 and a second Fab capable of binding to the fusion peptide of SARS-CoV-2, wherein the first Fab comprises a sequence comprising any one or more of preferred SEQ ID Numbers: 029, 136, 256, 372, 432 or 573 and wherein the second Fab comprises a sequence comprising any one or more of preferred SEQ ID Numbers: 639, 640, 641, 642, 643 to 644. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose an anti-SARS-CoV-2 bispecific antibody or an antigen binding fragment thereof, having a first Fab capable of binding to the stem helix of SARS-CoV-2 and a second Fab capable of binding to the fusion peptide of SARS-CoV-2, wherein the first Fab comprises a sequence comprising any one or more of preferred SEQ ID Numbers: 029, 136, 256, 372, 432 or 573 and wherein the second Fab comprises a sequence comprising any one or more of preferred SEQ ID Numbers: 645, 646, 647, 648, 649 to 650. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose an anti-SARS-CoV-2 bispecific antibody or an antigen binding fragment thereof, having a first Fab capable of binding to the stem helix of SARS-CoV-2 and a second Fab also capable of binding to the stem helix of SARS-CoV-2, wherein the first Fab comprises a sequence comprising any one or more of preferred SEQ ID Numbers: 029, 136, 256, 372, 432 or 573 and wherein the second Fab comprises a sequence comprising any one or more of preferred SEQ ID Numbers: 651, 652, 653, 654, 655 to 656. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose an anti-SARS-CoV-2 bispecific antibody or an antigen binding fragment thereof, having a first Fab capable of binding to the stem helix of SARS-CoV-2 and a second Fab also capable of binding to the stem helix of SARS-CoV-2, wherein the first Fab comprises a sequence comprising any one or more of preferred SEQ ID Numbers: 029, 136, 256, 372, 432 or 573 and wherein the second Fab comprises a sequence comprising any one or more of preferred SEQ ID Numbers: 657, 658, 659, 660, 661 to 662. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose a bispecific antibody comprising a First Fab and a Second Fab, wherein the First Fab comprises any one or more of sequences shown in preferred SEQ ID Numbers: 029, 136 or 256, and wherein the Second Fab comprises any one or more of sequences shown in preferred SEQ ID Numbers: 660, 661, or 662.

In a preferred embodiment, we disclose a bispecific antibody comprising a First Fab and a Second Fab, wherein the First Fab comprises any one or more of sequences shown in preferred SEQ ID Numbers: 029, 136 or 256, and wherein the Second Fab comprises any one or more of sequences shown in preferred SEQ ID Numbers: 642, 643, or 644.

In a preferred embodiment, we disclose a bispecific antibody comprising a First Fab and a Second Fab, wherein the First Fab comprises any one or more of sequences shown in preferred SEQ ID Numbers: 029, 136 or 256, and wherein the Second Fab comprises any one or more of sequences shown in preferred SEQ ID Numbers: 648, 649, or 650.

In a preferred embodiment, we disclose a bispecific antibody comprising a First Fab and a Second Fab, wherein the First Fab comprises any one or more of sequences shown in preferred SEQ ID Numbers: 029, 136 or 256, and wherein the Second Fab comprises any one or more of sequences shown in preferred SEQ ID Numbers: 654, 655, or 656.

In a preferred embodiment, we disclose a bispecific antibody comprising a First Fab and a Second Fab, wherein the First Fab comprises any one or more of sequences shown in preferred SEQ ID Numbers: 657, 658, or 659, and wherein the Second Fab comprises any one or more of sequences shown in preferred SEQ ID Numbers: 372, 432 or 573.

In a preferred embodiment, we disclose a bispecific antibody comprising a First Fab and a Second Fab, wherein the First Fab comprises any one or more of sequences shown in preferred SEQ ID Numbers: 639, 640, or 641, and wherein the Second Fab comprises any one or more of sequences shown in preferred SEQ ID Numbers: 372, 432 or 573.

In a preferred embodiment, we disclose a bispecific antibody comprising a First Fab and a Second Fab, wherein the First Fab comprises any one or more of sequences shown in preferred SEQ ID Numbers: 645, 646, or 647, and wherein the Second Fab comprises any one or more of sequences shown in preferred SEQ ID Numbers: 372, 432 or 573.

In a preferred embodiment, we disclose a bispecific antibody comprising a First Fab and a Second Fab, wherein the First Fab comprises any one or more of sequences shown in preferred SEQ ID Numbers: 651, 652 or 653, and wherein the Second Fab comprises any one or more of sequences shown in preferred SEQ ID Numbers: 372, 432 or 573.

The present invention also provides construction, expression, purification methods for a bispecific antibody having a function of binding to the stem helix of SARS-CoV-2, and use of the bispecific antibody in the field of medicine and especially in the prevention and/or treatment of SARS-CoV-2 infections.

### CLAUSES

The following numbered clauses each represent preferred embodiments of the invention and are part of the description.

### Clause 26

An anti-coronavirus, preferably anti-SARS-CoV-2 bispecific antibody or an antigen binding fragment thereof, having a first Fab capable of binding to the stem helix of a coronavirus, preferably SARS-CoV-2 and a second Fab capable of binding to the fusion peptide of a coronavirus, preferably SARS-CoV-2, wherein the first Fab comprises a sequence comprising any one or more of preferred SEQ ID Numbers: 029, 136, 256, 372, 432 or 573 and wherein the second Fab comprises a sequence comprising any one or more of preferred SEQ ID Numbers: 639, 640, 641, 642, 643 to 644.

### Clause 27

A bispecific antibody or an antigen binding fragment thereof according to Clause 26, comprising a first Fab capable of binding to the stem helix of a coronavirus, preferably SARS-CoV-2, and a second Fab capable of binding to the fusion peptide of a coronavirus, preferably SARS-CoV-2, wherein the first Fab comprises a sequence comprising any one or more of preferred SEQ ID Numbers: 029, 136, 256, 372, 432 and/or 573 and wherein the second Fab comprises a sequence comprising any one or more of preferred SEQ ID Numbers: 639, 640, 641, 642, 643 and/or 644.

### Clause 28

A bispecific antibody or an antigen binding fragment thereof according to Clauses 26 or 27, comprising a first Fab specifically binding to the stem helix of a coronavirus, preferably SARS-CoV-2, and a second Fab specifically binding to the fusion peptide of a coronavirus, preferably SARS-CoV-2, characterized in that:
a.) the first Fab comprises a heavy chain variable region comprising as CDRs, a Heavy Chain CDR1 region of SEQ ID NO: 029, a Heavy Chain CDR2 region of SEQ ID NO: 136 and a Heavy Chain CDR3 region of SEQ ID NO: 256 and a light chain variable region comprising as CDRs a Light Chain CDR1 region of SEQ ID NO: 372, a Light Chain CDR2 region of SEQ ID NO: 432 and a Light Chain CDR3 region of SEQ ID NO: 573, and
b.) the second Fab comprises a heavy chain variable region comprising as CDRs, a Heavy Chain CDR1 region of SEQ ID NO: 639, a Heavy Chain CDR2 region of SEQ ID NO: 640 and a Heavy Chain CDR3 region of SEQ ID NO: 641 and a light chain variable region comprising as CDRs a Light Chain CDR1 region of SEQ ID NO: 642, a Light Chain CDR2 region of SEQ ID NO: 643 and a Light Chain CDR3 region of SEQ ID NO: 644.

### Clause 29

A bispecific antibody or an antigen binding fragment thereof according to any of Clauses 26 to 28, comprising:
a) A light chain comprising VL-CL domains and a heavy chain comprising VH-CH1-CH2-CH3 domains of an antibody or an antigen binding fragment thereof, capable of binding to the stem helix of a coronavirus, preferably SARS-CoV-2, and
b) A light chain comprising VL-CL domains and a heavy chain comprising VH-CH1-CH2-CH3 domains of an antibody or an antigen binding fragment thereof, capable of binding to the fusion peptide of a coronavirus, preferably SARS-CoV-2,
wherein the constant domains CL and CH1 from the antibody or an antigen binding fragment thereof that is capable of binding to the stem helix of a coronavirus, preferably SARS-CoV-2, are replaced by each other.

### Clause 30

A bispecific antibody or an antigen binding fragment thereof according to Clause 29, wherein the VH domain of the antibody or the antigen binding fragment thereof that is capable of binding to the stem helix of a coronavirus, preferably SARS-CoV-2, comprises as CDRs, a Heavy Chain CDR1 region of SEQ ID NO: 029, a Heavy Chain CDR2 region of SEQ ID NO: 136 and a Heavy Chain CDR3 region of SEQ ID NO: 256 and the VL domain of the antibody or the antigen binding fragment thereof that is capable of binding to the stem helix of a coronavirus, preferably SARS-CoV-2, comprises as CDRs a Light Chain CDR1 region of SEQ ID NO: 372, a Light Chain CDR2 region of SEQ ID NO: 432 and a Light Chain CDR3 region of SEQ ID NO: 573, and
wherein the VH domain of the antibody or the antigen binding fragment thereof that is capable of binding to the fusion peptide of a coronavirus, preferably SARS-CoV-2 comprises as CDRs, a Heavy Chain CDR1 region of SEQ ID NO: 639, a Heavy Chain CDR2 region of SEQ ID NO: 640 and a Heavy Chain CDR3 region of SEQ ID NO: 641 and the VL domain of the antibody or the antigen binding fragment thereof that is capable of binding to the fusion peptide of a coronavirus, preferably SARS-CoV-2, comprises as CDRs a Light Chain CDR1 region of SEQ ID NO: 642, a Light Chain CDR2 region of SEQ ID NO: 643 and a Light Chain CDR3 region of SEQ ID NO: 644.

### Clause 31

A bispecific antibody or an antigen binding fragment thereof according to any of Clauses 26 to 28, comprising:
a) A light chain comprising VL-CL domains and a heavy chain comprising VH-CH1-CH2-CH3 domains of an antibody, capable of binding to the stem helix of a coronavirus, preferably SARS-CoV-2; and
b) A light chain comprising VL-CL domains and a heavy chain comprising VH-CH1-CH2-CH3 domains of an antibody, capable of binding to the fusion peptide of a coronavirus, preferably SARS-CoV-2, wherein constant domains CL and CH1 from the antibody that is capable of binding to the fusion peptide of a coronavirus, preferably SARS-CoV-2 are replaced by each other.

### Clause 32

A bispecific antibody or an antigen binding fragment thereof according to Clause 31,
wherein the VH domain of the antibody or the antigen binding fragment thereof that is capable of binding to the stem helix of a coronavirus, preferably SARS-CoV-2, comprises as CDRs, a Heavy Chain CDR1 region of SEQ ID NO: 029, a Heavy Chain CDR2 region of SEQ ID NO: 136 and a Heavy Chain CDR3 region of SEQ ID NO: 256 and the VL domain of the antibody capable of binding to the stem helix of a coronavirus, preferably SARS-CoV-2, comprises as CDRs a Light Chain CDR1 region of SEQ ID NO: 372, a Light Chain CDR2 region of SEQ ID NO: 432 and a Light Chain CDR3 region of SEQ ID NO: 573, and
wherein the VH domain of the antibody capable of binding to the fusion peptide of a coronavirus, preferably SARS-CoV-2, comprises as CDRs, a Heavy Chain CDR1 region of SEQ ID NO: 639, a Heavy Chain CDR2 region of SEQ ID NO: 640 and a Heavy Chain CDR3 region of SEQ ID NO: 641 and the VL domain of the antibody capable of binding to the fusion peptide of a coronavirus, preferably SARS-CoV-2, comprises as CDRs a Light Chain CDR1 region of SEQ ID NO: 642, a Light Chain CDR2 region of SEQ ID NO: 643 and a Light Chain CDR3 region of SEQ ID NO: 644.

### Clause 33

A bispecific antibody or an antigen binding fragment thereof according to any of Clauses 26 to 28, comprising:
a) A light chain comprising VL-CL domains and a heavy chain comprising VH-CH1-CH2-CH3 domains of an antibody, capable of binding to the stem helix of a coronavirus, preferably SARS-CoV-2; and
b) A light chain comprising VL-CL domains and a heavy chain comprising VH-CH1-CH2-CH3 domains of an antibody, capable of binding to the fusion peptide of a coronavirus, preferably SARS-CoV-2, wherein domains VL-CL and VH-CH1 from the antibody that specifically binds to the fusion peptide of a coronavirus, preferably SARS-CoV-2, are replaced by each other.

### Clause 34

A bispecific antibody or an antigen binding fragment thereof according to any of Clauses 26 to 33, wherein the first Fab comprises a heavy chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 629.

### Clause 35

A bispecific antibody or an antigen binding fragment thereof according to any of Clauses 26 to 33, wherein the second Fab comprises a heavy chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 663.

### Clause 36

A bispecific antibody or an antigen binding fragment thereof according to any of Clauses 26 to 33, wherein the first Fab comprises a light chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 630.

### Clause 37

A bispecific antibody or an antigen binding fragment thereof according to any of Clauses 26 to 33, wherein the second Fab comprises a light chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 664.

### Clause 38

A bispecific antibody or an antigen binding fragment thereof according to any of Clauses 26 to 33, wherein the bispecific antibody or an antigen binding fragment thereof is selected from anyone of the group comprising a full-length antibody, a Fab, modified Fab, Fab′, modified Fab′, F(ab′)2, Fv, single domain antibodies, scFv, scFv-Fc, bi, tri or tetra-valent antibodies, Bis-scFv, diabodies, triabodies, tetrabodies and epitope binding fragments thereof.

### Clause 39

A method for the production of a bispecific antibody or an antigen binding fragment thereof according to any of Clauses 26 to 38, comprising a) culturing a host cell, comprising an expression vector, comprising a polynucleotide encoding a bispecific antibody or an antibody fragment according to any one of the preceding Clauses under conditions which permit the production of said bispecific antibody or an antigen binding fragment thereof, and b) isolating said bispecific antibody or an antigen binding fragment thereof.

### Clause 40

A nucleic acid molecule comprising a nucleic acid sequence, wherein the nucleic acid sequence encodes the bispecific antibody or antigen binding fragment thereof, a heavy chain variable region and/or a light chain variable region of the bispecific antibody or an antigen binding fragment thereof according to any of Clauses 26 to 38, preferably wherein the nucleic acid molecule is an isolated nucleic acid molecule.

### Clause 41

A nucleic acid molecule comprising a nucleic acid sequence, wherein the nucleic acid sequence encodes the bispecific antibody or antigen binding fragment thereof, a heavy chain variable region and/or light chain variable region of the bispecific antibody or an antigen binding fragment thereof according to any of Clauses 26 to 38 and Clause 40, wherein the nucleic acid sequence comprises at least anyone of:
(i) a first nucleic acid sequence encoding the amino acid sequence as set forth in SEQ ID NO. 029, or a first nucleic acid sequence encoding the amino acid sequence having at most 1 or 2 amino acids different from SEQ ID NO. 029,
(ii) a second nucleic acid sequence encoding the amino acid sequence as set forth in SEQ ID NO. 136, or a second nucleic acid sequence encoding the amino acid sequence having at most 1, 2, 3, 4, or 5 amino acids different from SEQ ID NO. 136,
(iii) a third nucleic acid sequence encoding the amino acid sequence as set forth in SEQ ID NO. 256, or a third nucleic acid sequence encoding the amino acid sequence having at most 1, 2 or 3 amino acids different from SEQ ID NO. 256,
(iv) a fourth nucleic acid sequence encoding the amino acid sequence as set forth in SEQ ID NO. 372, or a fourth nucleic acid sequence encoding the amino acid sequence having at most 1, 2, 3 or 4 amino acids different from SEQ ID NO. 372,
(v) a fifth nucleic acid sequence encoding the amino acid sequence as set forth in SEQ ID NO. 432, or a fifth nucleic acid sequence encoding the amino acid sequence having at most 1 or 2 amino acids different from SEQ ID NO. 432,
(vi) a sixth nucleic acid sequence encoding the amino acid sequence as set forth in SEQ ID NO. 573, or a sixth nucleic acid sequence encoding the amino acid sequence having at most 1, 2, 3, or 4 amino acids different from SEQ ID NO. 573,
(vii) a seventh nucleic acid sequence encoding the amino acid sequence as set forth in SEQ ID NO. 639, or a seventh nucleic acid sequence encoding the amino acid sequence having at most 1 or 2 amino acids different from SEQ ID NO. 639,
(viii) an eighth nucleic acid sequence encoding the amino acid sequence as set forth in SEQ ID NO. 640, or an eighth nucleic acid sequence encoding the amino acid sequence having at most 1, 2, 3, 4, or 5 amino acids different from SEQ ID NO. 640,
(ix) a nineth nucleic acid sequence encoding the amino acid sequence as set forth in SEQ ID NO. 641, or a nineth nucleic acid sequence encoding the amino acid sequence having at most 1, 2, 3, or 4 amino acids different from SEQ ID NO. 641,
(x) a tenth nucleic acid sequence encoding the amino acid sequence as set forth in SEQ ID NO. 642, or a tenth nucleic acid sequence encoding the amino acid sequence having at most 1, 2, 3, or 4 amino acids different from SEQ ID NO. 642,
(xi) an eleventh nucleic acid sequence encoding the amino acid sequence as set forth in SEQ ID NO. 643, or an eleventh nucleic acid sequence encoding the amino acid sequence having at most 1 or 2 amino acids different from SEQ ID NO. 643, and
(xii) a twelfth nucleic acid sequence encoding the amino acid sequence as set forth in SEQ ID NO. 644, or a twelfth nucleic acid sequence encoding the amino acid sequence having at most 1, 2, or 3 amino acids different from SEQ ID NO. 644.

### Clause 42

A bispecific antibody or an antigen binding fragment thereof according to any of Clauses 26 to 38 for use in a method of treating a coronavirus infection in a subject.

### Clause 43

A composition comprising the bispecific antibody or an antigen binding fragment thereof according to any of Clauses 26 to 38.

### Clause 44

A composition according to Clause 43 further comprising a second medicament, for simultaneous, separate, or sequential administration.

### Clause 45

A composition according to Clause 44, wherein the second medicament comprises a second antibody or antigen binding fragment thereof.

### Clause 46

A composition according to Clause 44, wherein the second medicament comprises a bispecific antibody or bispecific antigen binding fragment thereof.

### Clause 47

A composition according to Clause 43, wherein the composition further comprises a pharmaceutically acceptable excipient or carrier.

### Clause 48

A composition according to Clause 43, for use in inhibiting and/or treating a coronavirus infection in a subject, preferably, a SARS-CoV-1 or SARS-CoV-2 infection in a subject, comprising an effective amount of the bispecific antibody or an antigen binding fragment thereof, nucleic acid molecule, or vector, and optionally a pharmaceutically acceptable excipient or carrier.

### Clause 49

The composition of Clause 43, for use as a medicament.

### Clause 50

An inhaler device comprising the composition of Clause 43.

### Clause 51

A bispecific antibody or an antigen binding fragment thereof according to any of Clauses 26 to 38, wherein the bispecific antibody or an antigen binding fragment thereof is bivalent.

### Clause 52

A bispecific antibody or an antigen binding fragment thereof according to any of Clauses 26 to 38, wherein the bispecific antibody or antigen-binding fragment thereof is capable of binding to the stem helix and/or the fusion peptide of at least one of an α-coronavirus, β-coronavirus, γ-coronavirus, and δ-coronavirus, preferably at least an α-coronavirus and/or a β-coronavirus.

### CLAUSES

The following numbered clauses each represent preferred embodiments of the invention and are part of the description.

### Clause 53

An anti-coronavirus, preferably anti-SARS-CoV-2 bispecific antibody or an antigen binding fragment thereof, having a first Fab capable of binding to the stem helix of a coronavirus, preferably SARS-CoV-2 and a second Fab capable of binding to the fusion peptide of a coronavirus, preferably SARS-CoV-2, wherein the first Fab comprises a sequence comprising any one or more of preferred SEQ ID Numbers: 029, 136, 256, 372, 432, or 573 and wherein the second Fab comprises a sequence comprising any one or more of preferred SEQ ID Numbers: 645, 646, 647, 648, 649 to 650.

### Clause 54

A bispecific antibody or an antigen binding fragment thereof according to Clause 53, comprising a first Fab capable of binding to the stem helix of a coronavirus, preferably SARS-CoV-2, and a second Fab capable of binding to the fusion peptide of a coronavirus, preferably SARS-CoV-2, wherein the first Fab comprises a sequence comprising any one or more of preferred SEQ ID Numbers: 029, 136, 256, 372, 432, and/or 573 and wherein the second Fab comprises a sequence comprising any one or more of preferred SEQ ID Numbers: 645, 646, 647, 648, 649 and/or 650.

### Clause 55

A bispecific antibody or an antigen binding fragment thereof according to Clauses 53 or 54, comprising a first Fab specifically binding to the stem helix of a coronavirus, preferably SARS-CoV-2, and a second Fab specifically binding to the fusion peptide of a coronavirus, preferably SARS-CoV-2, characterized in that:
a.) the first Fab comprises a heavy chain variable region comprising as CDRs, a Heavy Chain CDR1 region of SEQ ID NO: 029, a Heavy Chain CDR2 region of SEQ ID NO: 136 and a Heavy Chain CDR3 region of SEQ ID NO: 256 and a light chain variable region comprising as CDRs a Light Chain CDR1 region of SEQ ID NO: 372, a Light Chain CDR2 region of SEQ ID NO: 432 and a Light Chain CDR3 region of SEQ ID NO: 573, and
b.) the second Fab comprises a heavy chain variable region comprising as CDRs, a Heavy Chain CDR1 region of SEQ ID NO: 645, a Heavy Chain CDR2 region of SEQ ID NO: 646 and a Heavy Chain CDR3 region of SEQ ID NO: 647 and a light chain variable region comprising as CDRs a Light Chain CDR1 region of SEQ ID NO: 648, a Light Chain CDR2 region of SEQ ID NO: 649 and a Light Chain CDR3 region of SEQ ID NO: 650.

### Clause 56

A bispecific antibody or an antigen binding fragment thereof according to any of Clauses 53 to 55, comprising:
a) A light chain comprising VL-CL domains and heavy chain comprising VH-CH1-CH2-CH3 domains of an antibody or an antigen binding fragment thereof, capable of binding to the stem helix of a coronavirus, preferably SARS-CoV-2, and
b) A light chain comprising VL-CL domains and heavy chain comprising VH-CH1-CH2-CH3 domains of an antibody or an antigen binding fragment thereof, capable of binding to the fusion peptide of a coronavirus, preferably SARS-CoV-2, wherein constant domains CL and CH1 from the antibody or an antigen binding fragment thereof that is capable of binding to the stem helix of a coronavirus, preferably SARS-CoV-2, are replaced by each other.

### Clause 57

A bispecific antibody or an antigen binding fragment thereof according to Clause 56,
wherein the VH domain of the antibody or the antigen binding fragment thereof that is capable of binding to the stem helix of a coronavirus, preferably SARS-CoV-2, comprises as CDRs, a Heavy Chain CDR1 region of SEQ ID NO: 029, a Heavy Chain CDR2 region of SEQ ID NO: 136 and a Heavy Chain CDR3 region of SEQ ID NO: 256 and the VL domain of the antibody or the antigen binding fragment thereof that is capable of binding to the stem helix of a coronavirus, preferably SARS-CoV-2, comprises as CDRs a Light Chain CDR1 region of SEQ ID NO: 372, a Light Chain CDR2 region of SEQ ID NO: 432 and a Light Chain CDR3 region of SEQ ID NO: 573, and
wherein the VH domain of the antibody or the antigen binding fragment thereof that is capable of binding to the fusion peptide of a coronavirus, preferably SARS-CoV-2, comprises as CDRs, a Heavy Chain CDR1 region of SEQ ID NO: 645, a Heavy Chain CDR2 region of SEQ ID NO: 646 and a Heavy Chain CDR3 region of SEQ ID NO: 647 and the VL domain of the antibody or the antigen binding fragment thereof that is capable of binding to the fusion peptide of a coronavirus, preferably SARS-CoV-2, comprises as CDRs a Light Chain CDR1 region of SEQ ID NO: 648, a Light Chain CDR2 region of SEQ ID NO: 649 and a Light Chain CDR3 region of SEQ ID NO: 650.

### Clause 58

A bispecific antibody, or an antigen binding fragment thereof according to any of Clauses 53 to 55, comprising:
a) A light chain comprising VL-CL domains and heavy chain comprising VH-CH1-CH2-CH3 domains of an antibody, capable of binding to the stem helix of a coronavirus, preferably SARS-CoV-2; and
b) A light chain comprising VL-CL domains and heavy chain comprising VH-CH1-CH2-CH3 domains of an antibody, capable of binding to the fusion peptide of a coronavirus, preferably SARS-CoV-2, wherein constant domains CL and CH1 from the antibody that is capable of binding to the fusion peptide of a coronavirus, preferably SARS-CoV-2 are replaced by each other.

### Clause 59

A bispecific antibody or an antigen binding fragment thereof according to Clause 58,
wherein the VH domain of the antibody or the antigen binding fragment thereof that is capable of binding to the stem helix of a coronavirus, preferably SARS-CoV-2, comprises as CDRs, a Heavy Chain CDR1 region of SEQ ID NO: 029, a Heavy Chain CDR2 region of SEQ ID NO: 136 and a Heavy Chain CDR3 region of SEQ ID NO: 256 and the VL domain of the antibody capable of binding to the stem helix of a coronavirus, preferably SARS-CoV-2, comprises as CDRs a Light Chain CDR1 region of SEQ ID NO: 372, a Light Chain CDR2 region of SEQ ID NO: 432 and a Light Chain CDR3 region of SEQ ID NO: 573, and
wherein the VH domain of the antibody capable of binding to the fusion peptide of a coronavirus, preferably SARS-CoV-2, comprises as CDRs, a Heavy Chain CDR1 region of SEQ ID NO: 645, a Heavy Chain CDR2 region of SEQ ID NO: 646 and a Heavy Chain CDR3 region of SEQ ID NO: 647 and the VL domain of the antibody capable of binding to the fusion peptide of a coronavirus, preferably SARS-CoV-2, comprises as CDRs a Light Chain CDR1 region of SEQ ID NO: 648, a Light Chain CDR2 region of SEQ ID NO: 649 and a Light Chain CDR3 region of SEQ ID NO: 650.

### Clause 60

A bispecific antibody or an antigen binding fragment thereof according to any of Clauses 53 to 55, comprising:
a) A light chain comprising VL-CL domains and a heavy chain comprising VH-CH1-CH2-CH3 domains of an antibody, capable of binding to the stem helix of a coronavirus, preferably SARS-CoV-2; and
b) A light chain comprising VL-CL domains and a heavy chain comprising VH-CH1-CH2-CH3 domains of an antibody, capable of binding to the fusion peptide of a coronavirus, preferably SARS-CoV-2, wherein domains VL-CL and VH-CH1 from the antibody that specifically binds to the fusion peptide of a coronavirus, preferably SARS-CoV-2, are replaced by each other.

### Clause 61

A bispecific antibody or an antigen binding fragment thereof according to any of Clauses 53 to 60, wherein the first Fab comprises a heavy chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 629.

### Clause 62

A bispecific antibody or an antigen binding fragment thereof according to any of Clauses 53 to 60, wherein the second Fab comprises a heavy chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 665.

### Clause 63

A bispecific antibody or an antigen binding fragment thereof according to any of Clauses 53 to 60, wherein the first Fab comprises a light chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 630.

### Clause 64

A bispecific antibody or an antigen binding fragment thereof according to any of Clauses 53 to 60, wherein the second Fab comprises a light chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 666.

### Clause 65

A bispecific antibody or an antigen binding fragment thereof according to any of Clauses 53 to 60, wherein the bispecific antibody or antigen binding fragment thereof is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

### Clause 66

A method for the production of a bispecific antibody or an antigen binding fragment thereof according to any of Clauses 53 to 65, comprising a) culturing a host cell, comprising an expression vector, comprising a polynucleotide encoding a bispecific antibody or an antibody fragment according to any one of the preceding Clauses under conditions which permit the production of said bispecific antibody or an antigen binding fragment thereof, and b) isolating said bispecific antibody or an antigen binding fragment thereof.

### Clause 67

A nucleic acid molecule comprising a nucleic acid sequence, wherein the nucleic acid sequence encodes the bispecific antibody or antigen binding fragment thereof, a heavy chain variable region and/or a light chain variable region of the bispecific antibody or an antigen binding fragment thereof according to any of Clauses 53 to 65, preferably wherein the nucleic acid molecule is an isolated nucleic acid molecule.

### Clause 68

A nucleic acid molecule comprising a nucleic acid sequence, wherein the nucleic acid sequence encodes the bispecific antibody or antigen binding fragment thereof, a heavy chain variable region and/or light chain variable region of the bispecific antibody or the antigen binding fragment thereof according to any of Clauses 53 to 65 and Clause 67, wherein the nucleic acid sequence comprises at least anyone of:
(i) a first nucleic acid sequence encoding the amino acid sequence as set forth in SEQ ID NO. 029, or a first nucleic acid sequence encoding the amino acid sequence having at most 1 or 2 amino acids different from SEQ ID NO. 029,
(ii) a second nucleic acid sequence encoding the amino acid sequence as set forth in SEQ ID NO. 136, or a second nucleic acid sequence encoding the amino acid sequence having at most 1, 2, 3, 4, or 5 amino acids different from SEQ ID NO. 136,
(iii) a third nucleic acid sequence encoding the amino acid sequence as set forth in SEQ ID NO. 256, or a third nucleic acid sequence encoding the amino acid sequence having at most 1, 2 or 3 amino acids different from SEQ ID NO. 256,
(iv) a fourth nucleic acid sequence encoding the amino acid sequence as set forth in SEQ ID NO. 372, or a fourth nucleic acid sequence encoding the amino acid sequence having at most 1, 2, 3 or 4 amino acids different from SEQ ID NO. 372,
(v) a fifth nucleic acid sequence encoding the amino acid sequence as set forth in SEQ ID NO. 432, or a fifth nucleic acid sequence encoding the amino acid sequence having at most 1 or 2 amino acids different from SEQ ID NO. 432,
(vi) a sixth nucleic acid sequence encoding the amino acid sequence as set forth in SEQ ID NO. 573, or a sixth nucleic acid sequence encoding the amino acid sequence having at most 1, 2, 3, or 4 amino acids different from SEQ ID NO. 573,
(vii) a seventh nucleic acid sequence encoding the amino acid sequence as set forth in SEQ ID NO. 645, or a seventh nucleic acid sequence encoding the amino acid sequence having at most 1 or 2 amino acids different from SEQ ID NO. 645,
(viii) an eighth nucleic acid sequence encoding the amino acid sequence as set forth in SEQ ID NO. 646, or an eighth nucleic acid sequence encoding the amino acid sequence having at most 1, 2, 3, 4, or 5 amino acids different from SEQ ID NO. 646,
(ix) a nineth nucleic acid sequence encoding the amino acid sequence as set forth in SEQ ID NO. 647, or a nineth nucleic acid sequence encoding the amino acid sequence having at most 1, 2, 3, 4, or 5 amino acids different from SEQ ID NO. 647,
(x) a tenth nucleic acid sequence encoding the amino acid sequence as set forth in SEQ ID NO. 648, or a tenth nucleic acid sequence encoding the amino acid sequence having at most 1, 2, or 3 amino acids different from SEQ ID NO. 648,
(xi) an eleventh nucleic acid sequence encoding the amino acid sequence as set forth in SEQ ID NO. 649, or an eleventh nucleic acid sequence encoding the amino acid sequence having at most 1 or 2 amino acids different from SEQ ID NO. 649, and
(xii) a twelfth nucleic acid sequence encoding the amino acid sequence as set forth in SEQ ID NO. 650, or a twelfth nucleic acid sequence encoding the amino acid sequence having at most 1, 2, or 3 amino acids different from SEQ ID NO. 650.

### Clause 69

A bispecific antibody or an antigen binding fragment thereof according to any of Clauses 53 to 65 for use in a method of treating a coronavirus infection in a subject.

### Clause 70

A composition comprising the bispecific antibody and/or an antigen binding fragment thereof according to any of Clauses 53 to 65.

### Clause 71

A composition according to Clause 70 further comprising a second medicament, for simultaneous, separate, or sequential administration.

### Clause 72

A composition according to Clause 71, wherein the second medicament comprises a second antibody or antigen binding fragment thereof.

### Clause 73

A composition according to Clause 71, wherein the second medicament comprises a bispecific antibody or bispecific antigen binding fragment thereof.

### Clause 74

A composition according to Clause 70, wherein the composition further comprises a pharmaceutically acceptable excipient or carrier.

### Clause 75

A composition according to Clause 70, for use in inhibiting and/or treating a coronavirus infection in a subject, preferably, a SARS-CoV-1 or SARS-CoV-2 infection in a subject, comprising an effective amount of the bispecific antibody or an antigen binding fragment thereof, nucleic acid molecule, or vector, and optionally a pharmaceutically acceptable excipient or carrier.

### Clause 76

The composition of Clause 70, for use as a medicament.

### Clause 77

An inhaler device comprising the composition of Clause 70.

### Clause 78

A bispecific antibody or an antigen binding fragment thereof according to any of Clauses 53 to 65, wherein the bispecific antibody or an antigen binding fragment thereof is bivalent.

### Clause 79

A bispecific antibody or an antigen binding fragment thereof according to any of Clauses 53 to 65, wherein the bispecific antibody or antigen binding fragment thereof is capable of binding to the stem helix and/or the fusion peptide of at least one of an α-coronavirus, β-coronavirus, γ-coronavirus, and δ-coronavirus, preferably at least an α-coronavirus and/or a β-coronavirus.

### DESCRIPTION OF THE DRAWINGS

FIG. 1. Survival after lethal challenge—CV3-25.

Kaplan-Meier survival curves of the intraperitoneal prophylactic treatment group. Animals (n=10 per group), treated with a dose titration with the control antibody, CV3-25, at day −1. At day 0, animals were infected with 10^{3.5} TCID₅₀ of SARS-CoV-2 Delta. A vehicle control group was included (PBS). Lines are nudged relative to the Y-axis to improve visual representation.

FIG. 2. Survival after lethal challenge—CV3-25.

Kaplan-Meier survival curves of the intranasal prophylactic treatment group. Animals (n=10 per group, except 15 mg/kg where n=8), treated with a dose titration with the control antibody, CV3-25, at day −1. At day 0, animals were infected with 10^{3.5} TCID₅₀ of SARS-CoV-2 Delta. A vehicle control group was included (PBS). Lines are nudged relative to the Y-axis to improve visual representation.

FIG. 3. Survival after lethal challenge—Antibody according to the invention.

Kaplan-Meier survival curves of the intraperitoneal prophylactic treatment group. Animals (n=10 per group, except 0.5 mg/kg where n=9), treated with a dose titration with the antibody having heavy chain CDR1 as SEQ ID NO: 029, heavy chain CDR2 as SEQ ID NO: 136, heavy chain CDR3 as SEQ ID NO: 256, a light chain CDR1 as SEQ ID NO: 372, a light chain CDR2 as SEQ ID NO: 432, and a light chain CDR3 as SEQ ID NO: 573, at day −1. At day 0, animals were infected with 10^{3.5} TCID₅₀ of SARS-CoV-2 Delta. A vehicle control group was included (PBS). Lines are nudged relative to the X and Y-axis to improve visual representation.

FIG. 4. Survival after lethal challenge—Antibody according to the invention.

Kaplan-Meier survival curves of the intranasal prophylactic treatment group. Animals (n=10 per group), treated with a dose titration with the antibody having heavy chain CDR1 as SEQ ID NO: 029, heavy chain CDR2 as SEQ ID NO: 136, heavy chain CDR3 as SEQ ID NO: 256, a light chain CDR1 as SEQ ID NO: 372, a light chain CDR2 as SEQ ID NO: 432, and a light chain CDR3 as SEQ ID NO: 573, at day −1. At day 0, animals were infected with 10^{3.5} TCID₅₀ of SARS-CoV-2 Delta. A vehicle control group was included (PBS). Lines are nudged relative to the Y-axis to improve visual representation.

FIG. 5. Bodyweight change—CV3-25.

Bodyweight change (%) relative to day 0, for the intraperitoneal prophylactic treatment group. Animals (n=10 per group) were treated with a dose titration of the control antibody, CV3-25, at day −1 intranasally. At day 0 animals were infected with 10^{3.5} TCI D₅₀ of SARS-CoV-2 Delta. A vehicle control group was included (PBS). Bars represent the 95% CI. of the mean. If a mouse died/was euthanized during follow-up of the study, the last observed bodyweight was carried forward.

FIG. 6. Bodyweight change—CV3-25.

Bodyweight change (%) relative to day 0, for the intranasal prophylactic treatment group. Animals (n=10 per group, except 15 mg/kg where n=8) were treated with a dose titration of the control antibody, CV3-25, at day −1 intranasally. At day 0 animals were infected with 10^{3.5} TCID₅₀ of SARS-CoV-2 Delta. A vehicle control group was included (PBS). Bars represent the 95% CI. of the mean. If a mouse died/was euthanized during follow-up of the study, the last observed bodyweight was carried forward.

FIG. 7. Bodyweight change—Antibody according to the invention.

Bodyweight change (%) relative to day 0, for the intraperitoneal prophylactic treatment group. Animals (n=10 per group, except 0.5 mg/kg where n=9) were treated with a dose titration of the antibody having heavy chain CDR1 as SEQ ID NO: 029, heavy chain CDR2 as SEQ ID NO: 136, heavy chain CDR3 as SEQ ID NO: 256, a light chain CDR1 as SEQ ID NO: 372, a light chain CDR2 as SEQ ID NO: 432, and a light chain CDR3 as SEQ ID NO: 573, at day −1 intranasally. At day 0 animals were infected with 10^{3.5} TCID₅₀ of SARS-CoV-2 Delta. A vehicle control group was included (PBS). Bars represent the 95% CI. of the mean. If a mouse died/was euthanized during follow-up of the study, the last observed bodyweight was carried forward.

FIG. 8. Bodyweight change—Antibody according to the invention.

Bodyweight change (%) relative to day 0, for the intranasal prophylactic treatment group. Animals (n=10 per group) were treated with a dose titration of the antibody having heavy chain CDR1 as SEQ ID NO: 029, heavy chain CDR2 as SEQ ID NO: 136, heavy chain CDR3 as SEQ ID NO: 256, a light chain CDR1 as SEQ ID NO: 372, a light chain CDR2 as SEQ ID NO: 432, and a light chain CDR3 as SEQ ID NO: 573, at day −1 intranasally. At day 0 animals were infected with 10^{3.5} TCID₅₀ of SARS-CoV-2 Delta. A vehicle control group was included (PBS). Bars represent the 95% CI. of the mean. If a mouse died/was euthanized during follow-up of the study, the last observed bodyweight was carried forward.

FIG. 9. Linear epitope mapping against Spike helix domain of alpha and beta coronaviruses.

Grey highlighted amino acids were identified as the epitope where the antibody is binding the Spike protein. Target sequences for the 7 tested strains are aligned by Clustal. * may contain multiple smaller parts of the epitope. Epitope footprints (B) and paratope hotspots (A) for the antibody against the SH domain of SARS-CoV-2. CDR regions for heavy and light chains are indicated.

### DETAILED DESCRIPTION OF THE INVENTION

For the purpose of clarity and a concise description, features may be described herein as part of the same or separate embodiments, however, it will be appreciated that the disclosure includes embodiments having combinations of all or some of the features described. References to “method for treatment”, “treatment method”, “antibody for use” and “use of an antibody in the manufacture of a medicament” can be used interchangeably and embodiments disclosed in relation to any one of those aspects also applies in relation to said other aspects. In other words, they all refer to medical treatments involving an antibody as disclosed herein.

### Prophylactic Treatment

The term ‘prophylactic treatment’, as used herein, includes reference to a treatment for preventing infection of an individual with a coronavirus, or preventing symptoms after infection with a coronavirus, or preventing severe symptoms after known infection with a coronavirus with or without symptoms, or preventing hospitalization and death after infection with a coronavirus.

Prevention of an infection is preferably performed by administration of an antibody as disclosed herein prior to coronavirus exposure i.e. pre-exposure prophylaxis.

‘Prophylactically’ therefore preferably means prior to virus exposure. Nonetheless, it may also involve administration after infection, for example for reducing the replication or spread, or increasing clearance of the virus i.e. post-exposure prophylaxis.

Infected individuals may present with no symptoms. Alternatively, infected individuals may present with symptoms.

Preferably, post-exposure prophylaxis involves administering an antibody as disclosed herein after coronavirus exposure to prevent symptomatic disease.

Preferably, post-exposure prophylaxis involves administering an antibody as disclosed herein after coronavirus exposure to prevent severe disease, in particular hospitalization.

In a treatment method of the invention, prophylactic treatment involves administration of antibodies against coronavirus at a point in time when the individual is not infected with coronavirus. Preferably, said antibodies bind to a conserved epitope of the spike protein of a coronavirus virion. In some embodiments, an individual in need thereof is not (yet) infected with coronavirus.

### Therapeutic Treatment

The term ‘therapeutic treatment’, as used herein, includes reference to treatment of a viral infection (including coronavirus disease) after viral infection has taken place. A viral infection involves the entry of the body by the virus, and/or the replication of the virus in the body and/or the spreading of the virus to cells, tissues or locations in the body that were previously uninfected. A viral infection may cause one or more disease, but may also be latent, in other words may reside in the body without causing symptoms or disease.

### Coronavirus

The term ‘coronavirus’, as used herein, includes reference to a positive-sense single-stranded RNA virus belonging to the family of Coronaviridae.

Preferably, an antibody as disclosed herein is capable of specifically binding to the stem helix of a coronavirus, in particular SARS-CoV-2.

Preferably, an antibody as disclosed herein is capable of specifically binding to the stem helix of SARS-CoV-2 variants of concern.

Preferably, the antibody as disclosed herein is capable of neutralizing a coronavirus, in particular SARS-CoV-2.

Preferably, an antibody as disclosed herein is capable of neutralizing at least one or more, preferably two or more, preferably three or more, preferably four or more, even more preferably five or more coronavirus subtypes.

The phrase “is capable of” as used in, means the ability of an antibody or an antigen-binding fragment thereof as described herein is enough to bind to at least a part of an antigen, such as the stem helix of a coronavirus, in particular SARS-CoV-2 and/or the fusion protein of a coronavirus, in particular SARS-CoV-2. The binding affinity or the binding ability of the antibody and/or the antigen-binding fragment thereof as described herein can be measured using any methods known by a person skilled in the art. The phrase “is capable of” as used here, encompasses but not limited to the phrase “specifically binding” or “specifically bind to”.

### Coronavirus Infection

The term ‘coronavirus infection’, as used herein, includes reference to the pathological or non-pathological, preferably pathological, entrance and residence of a coronavirus of any type in a human host. The infecting virus may replicate within the host, its cells or the cells of its microbiome. The infecting virus may or may not cause a disease, for example COVID-19. The infection may or may not be able to be detected by methods for virus infection detection known in the art. The infected individual may or may not be aware of the infection. Typical, but non-exclusive locations of the human body where, for example SARS-CoV-2, may be located in an infected individual, are the respiratory system and/or cells thereof and the cardiovascular system and/or cells thereof. The term ‘coronavirus infection’, as used herein, further includes reference to the entrance and residence of a part of a coronavirus of any type that is able to cause viral replication in a human host. The term ‘coronavirus infection’ encompasses symptoms or disease following the infection, e.g. COVID-19. The term ‘coronavirus infection’ encompasses ‘SARS-CoV-2 infection’.

### Epitope

An “epitope”, as used herein, includes reference to a moiety that is capable of binding to an antibody as disclosed herein with sufficiently high affinity to form a detectable antigen-antibody complex.

### Individual

The term ‘individual’, as used herein, includes reference to a mammal or human that is subject to, or a risk of suffering from viral infection. Infection may take place in any system, tissue or cell belonging to the host, including the host's microbiome. Coronavirus infection and the disease coronavirus may occur in individuals of all age groups and sexes. Nonetheless, preferably, the individual is a human, in particular an elderly human such as a human that is at least 60, 65, 70, 75, 80, or at least 85 years old, or that has an increased risk of infection because of occupation or living environment. Preferably, the individual is at risk of suffering from severe illness, for example COVID-19, once infected. In preferred embodiments, the individual has an underlying disease such as (i) a respiratory disease such as asthma, COPD, chronic bronchitis and lung emphysema, (ii) cardiovascular disease such as cardiac arrhythmia or individuals that have received cardiac surgery, (iii) diabetes, (iv) renal failure and/or (v) a disease affecting the immune system, for instance immunocompromised individuals, or higher risk of viral infection because of occupation.

The phrase ‘an individual’, as used herein, includes reference to a mammal such as a but not limited to a human that benefits from a specified therapy, for example, the phrase ‘an individual’ may encompass Non-human primates (NHP).

Preferably, the individual is a mammal, more preferably a human.

### Administering and Administration

The terms ‘administering’ and ‘administration’, as used herein, include reference to the provision of one or more drug and optionally one or more adjuvant with the aim to treat, cure, reduce, or prevent a disease or its symptoms in an individual, or to promote the individual's well-being. Preferred methods of administration of the antibody as disclosed herein include mucosal administration, preferably intranasal administration and oral inhalation.

### An Individual in Need Thereof

The phrase ‘an individual in need thereof’, as used herein, includes reference to a mammal such as a human that benefits from a specified therapy. A treatment method of the invention may be used prophylactically, the exhibition of symptoms or indications for coronavirus infection are not required. Individuals that are especially in need of the method or antibody for use of the invention, are individuals with an elevated risk of coronavirus infection, individuals with an elevated risk of developing severe symptoms (illness), for example COVID-19, and/or individuals with an elevated risk of dying from COVID-19. The person skilled in the art is aware of the risk factors for an elevated risk of coronavirus infection, an elevated risk of developing severe symptoms of coronavirus infection, and an elevated risk of dying from coronavirus infection.

### Antibody

The term ‘antibody’, as used herein, includes reference to an intact immunoglobulin, including monoclonal antibodies, such as chimeric, humanized or human monoclonal antibodies, or to a binding molecule comprising an antigen-binding domain (such as heavy chain CDRs 1-3 of a variable domain) of an antibody as disclosed herein or an antibody that competes with an antibody as disclosed herein for specific binding to the binding partner of the immunoglobulin.

In other words, functional fragments of antibodies are also encompassed by the term ‘antibody’. Antibodies are generally Y-shaped proteins. Within the antibody, constant domain and variable domains are generally present. The variable domain facilitates antigen binding. An antibody generally comprises two heavy chains and two light chains. Both the heavy chains and the light chains are partially constant and partially variable. Antibodies occur in a few classes: IgA, IgD, IgE, IgG and IgM. Preferably, the antibody of the invention is of the IgG, preferably IgG1, class. Some classes may be further subdivided into subclasses or isotypes. For example, the IgG class is subdivided into the subclasses IgG1, IgG2, IgG3 and IgG4. Preferably, the antibody of the invention is of the IgG, preferably IgG1, class. Antigen-binding regions, or antigen-binding fragments of an antibody, which are encompassed by the term ‘antibody’ and which are therefore part of the present invention, may include, for example, Fab, F(ab′), F(ab′)2, dAb, Fv, Fd, CDR fragments, diabodies, triabodies, tetrabodies, single-chain antibodies (scFv, scFv-Fc), bivalent single-chain antibodies, single-chain phage antibodies, (poly) peptides that contain at least a fragment of an immunoglobulin that is sufficient to confer specific antigen binding to the (poly) peptide, etc. The above fragments may be produced synthetically or by enzymatic or chemical cleavage of intact immunoglobulins or they may be genetically engineered by recombinant DNA techniques. The methods for production of antibodies and antigen-binding fragments are well-known to a person skilled in the art. The antibody may be conjugated or unconjugated. The antibody may be conjugated, linked, or otherwise physically or functionally associated with an effector moiety or tag, such as inter alia an enzyme, a liposome, a radioactive substance, a fluorophore, a toxic substance. Antibodies may have been stabilized, multimerized, humanized or otherwise manipulated.

Antibodies may be neutralizing, which includes reference to inhibition of a virus as measured by an in vitro neutralization assay, for instance in terms of viral host cell entry and/or viral replication. Neutralization can for example be achieved by inhibiting the attachment or adhesion of the virus to the cell surface, or by inhibition of the fusion of viral and cellular membranes following attachment of the virus to the target cell or by inhibiting viral egress from cells. Neutralization does not specify the method of neutralization. Preferably, the antibody is cross-neutralizing, which includes reference to the ability of the antibodies of the invention to bind a set of different molecules, preferably different molecules of different subtypes belonging to the Coronaviridae family.

The term “domain” or “region” as used herein, with reference to a part of an antibody, bispecific antibody, or an antigen binding fragment thereof, can be used interchangeably herein.

The phrase “antigen-binding fragments”, “antigen binding fragments”, “antigen fragment”, “a fragment of an antibody” or “antigen-binding molecule” as used herein, means a protein, polypeptide or molecular complex comprising or consisting of at least one complementarity determining region (CDR) that alone, or in combination with one or more additional CDRs and/or framework regions (FRs), as described herein, is capable of binding to a part of an antigen, including the stem helix of a coronavirus, in particular SARS-CoV-2, and/or the stem helix of a coronavirus, in particular SARS-CoV-2 and its variants of concern. The phrases “antigen-binding fragments”, “antigen binding fragments”, “antigen fragment”, “a fragment of an antibody” or “antigen-binding molecule” as used herein, can be used interchangeably. For instance, the phrase “antigen binding fragments” as used herein is to be used interchangeably with the phrase “antibody fragments” as used herein.

Antibodies comprise complementarity determining regions situated on the variable domains of the heavy chain and the light chain. The CDRs contribute to a large extent to the antigen binding site. Three CDRs can be distinguished, namely CDR1, CDR2 and CDR3. As each CDR can be located on either the light chain or the heavy chain, there are generally six CDRs for each antigen receptor that collectively contact the antigen: the light chain CDR1, the light chain CDR2, the light chain CDR3, the heavy chain CDR1, the heavy chain CDR2 and the heavy chain CDR3. The CDRs of type CDR3 are the most variable. The CDRs can be specific for linear epitopes, discontinuous epitopes, or conformational epitopes of proteins or protein fragments, either as present on the protein in its native conformation or, in some cases, as present on the proteins as denatured or activated. Epitopes may also consist of or comprise post-translational modifications of proteins. Antibodies of the invention that are of particular interest, are antibodies comprising CDRs that recognize coronavirus antigens, such as the spike protein and in particular the stem helix of the spike protein. The antibody as disclosed herein binds to a conserved epitope in the stem helix at the base of the spike protein of the SARS-CoV-2 virion. The epitope of the antibody as disclosed herein uses light and heavy chain CDR loops.

The antibody as disclosed herein can be used in isolated or non-isolated form.

Preferably, the compositions of the invention comprise a single anti-coronavirus antibody as disclosed herein.

Preferably, the antibody of the invention as disclosed herein can cross-neutralize coronavirus, in particular SARS-CoV-2.

Furthermore, the antibody as disclosed herein can be used alone or in a mixture comprising the antibody (or variant, fragment or bispecific thereof) as disclosed herein, and/or with other antibodies that bind to a coronavirus and have a coronavirus inhibiting effect. In other words, the antibody as disclosed herein can be used in combination, e.g., as a pharmaceutical composition or co-administration of compositions comprising two or more antibodies that specifically bind coronavirus. For example, antibodies having different, but complementary activities can be combined in a single therapy to achieve a desired therapeutic or prophylactic effect, but alternatively, antibodies having identical activities can also be combined in a single therapy to achieve a desired prophylactic or therapeutic effect. Optionally, the mixture further comprises at least one other therapeutic agent.

Preferably, the antibody as disclosed herein are a bispecific antibody and/or an antigen binding fragment thereof, wherein the bispecific antibody is capable of binding to the stem helix of a coronavirus and/or capable of binding to fusion peptide of coronavirus. The phrase “capable of binding” encompasses the phase “specifically binds to”, in other words, an antibody, a bispecific antibody and/or an antigen binding fragment thereof as disclosed herein are that is capable of binding to an epitope may also be capable of binding the other epitopes.

Preferably, the coronavirus as described herein includes SARS-CoV-2 and variants.

The phrase “are replaced by each other”, “are mutually interchanged”, “swap places with each other”, or “are traded for one another” as used herein, includes reference to at least a part of the heavy chain domains of an antibody or an antigen-binding fragment as described herein such as when a CH1 and/or VH, is substituted by at least a part of the light chain domains such as CL and/or VL to which said at least a part of the heavy chain domains are paired, and at the same instant, said at least a part of the light chain domains is substituted by said at least a part of the heavy chain domains. The phrase “are replaced by each other”, “are mutually interchanged”, “swap places with each other”, or “are traded for one another” are to be used interchangeably herein.

Preferably, the antibody as disclosed herein is a human antibody.

### Framework Regions

Antibodies also comprise framework regions, generally four framework regions (FR1, FR2, FR3 and FR4) on each of the variable heavy chain domain and variable light chain domain. The CDRs are situated between the framework regions (preferably in the order FR1-CDR1-FR2-CDR2-FR3-CDR3-FR4) and both the CDRs and framework regions together define a heavy chain variable domain (or region) and light chain variable domain (or region) which may also be referred to as an antigen-binding region or domain.

### IgG Antibody

The term ‘IgG antibody’, as used herein, includes reference to an antibody comprising two antigen-binding sites. IgG is the most common antibody in human serum. Heavy chains of IgG antibodies are of type γ, which can be subdivided in γ1, γ2, γ3 and 4. Preferably, the antibodies described herein are γ1 heavy chains. Light chains of IgG antibodies are of type λ or κ. Preferably, the antibodies described herein are A light chains.

Preferably, the antibody is an IgG antibody, preferably an IgG1 antibody.

### IgA Antibody

The term ‘IgA antibody’, as used herein, includes reference to an antibody comprising two to four antigen-binding sites. IgA is the most abundant antibody in mucosal secretions. An IgA antibody can be produced in monomeric, dimeric or secretory form, with two or four antigen-binding sites, respectively, and can exist as an IgA1 or IgA2 isotype. [Heavy chains of IgA antibodies are of type α, which can be subdivided in α1 and α2. Light chains of IgA antibodies are of type λ or κ.

Preferably, the antibody is an IgA antibody.

### IgM Antibody

The term ‘IgM antibody’, as used herein, includes reference to an antibody comprising ten to twelve antigen-binding sites. An IgM antibody can be produced in pentameric or hexameric form. Heavy chains of IgM antibodies are of type μ. Light chains of IgM antibodies are of type λ or κ.

Preferably, the antibody is an IgM antibody.

### Anti-Coronavirus Antibody

The term ‘anti-coronavirus antibody’, as used herein, includes reference to an antibody as disclosed herein, i.e. an antibody that can be used in a treatment method of the invention. Preferably, the anti-coronavirus antibody of the invention is able to treat different subtypes of coronavirus.

For the avoidance of doubt, the term ‘anti-coronavirus antibody’, can be used interchangeably with ‘coronavirus antibody’.

### A Composition

In a preferred embodiment, an antibody disclosed herein is the sole active ingredient in a composition administered for treatment, e.g., the antibody is provided in a composition as the sole active ingredient.

In a preferred embodiment, the active ingredients comprise an antibody disclosed herein, e.g., the antibody is provided either as a composition as the sole active ingredient in a composition administered for treatment or in a combination with another antibody.

In a preferred embodiment, a first antibody as disclosed herein is provided as a composition in combination with a second antibody, wherein the first antibody comprises a CDR sequence comprising any one of SEQ ID NO: 004 to 009, 015 to 020, 029 to 034, 039 to 044, 047 to 052, 065 to 070, 082 to 087, 103 to 108, 125 to 130, 136 to 141, 147 to 152, 191 to 196, 210 to 215, 221 to 226, 245 to 250, 256 to 261, 286 to 291, 297 to 302, 319 to 324, 330 to 335, 350 to 355, 372 to 377, 383 to 388, 415 to 420, 432 to 437, 443 to 448, 454 to 459, 465 to 470, 476 to 481, 508 to 513, 529 to 534, 540 to 545, 573 to 578, 601 to 606, 612 to 617, 623 to 628. More preferably wherein the first antibody comprises CDR sequences comprising any one or more of SEQ ID NO: 029, 136, 256, 372, 432 or 573, and the second antibody comprises CDR sequences comprising any one or more of SEQ ID NO: 639, 640, 641, 642, 643 or SEQ ID NO.: 644. Preferably wherein the composition is administered for prophylactic and/or therapeutic treatment.

In a preferred embodiment, a first antibody as disclosed herein is provided as a composition in combination with a second antibody, wherein the first antibody comprises a CDR sequence comprising any one of SEQ ID NO: 004 to 009, 015 to 020, 029 to 034, 039 to 044, 047 to 052, 065 to 070, 082 to 087, 103 to 108, 125 to 130, 136 to 141, 147 to 152, 191 to 196, 210 to 215, 221 to 226, 245 to 250, 256 to 261, 286 to 291, 297 to 302, 319 to 324, 330 to 335, 350 to 355, 372 to 377, 383 to 388, 415 to 420, 432 to 437, 443 to 448, 454 to 459, 465 to 470, 476 to 481, 508 to 513, 529 to 534, 540 to 545, 573 to 578, 601 to 606, 612 to 617, 623 to 628. More preferably wherein the first antibody comprises CDR sequences comprising any one or more of SEQ ID NO: 029, 136, 256, 372, 432 or 573, and the second antibody comprises CDR sequences comprising any one or more of SEQ ID NO: 645, 646, 647, 648, 649 or SEQ ID NO.: 650. Preferably wherein the composition is administered for prophylactic and/or therapeutic treatment.

In a preferred embodiment, a first antibody as disclosed herein is provided as a composition in combination with a second antibody, wherein the first antibody comprises a CDR sequence comprising any one of SEQ ID NO: 004 to 009, 015 to 020, 029 to 034, 039 to 044, 047 to 052, 065 to 070, 082 to 087, 103 to 108, 125 to 130, 136 to 141, 147 to 152, 191 to 196, 210 to 215, 221 to 226, 245 to 250, 256 to 261, 286 to 291, 297 to 302, 319 to 324, 330 to 335, 350 to 355, 372 to 377, 383 to 388, 415 to 420, 432 to 437, 443 to 448, 454 to 459, 465 to 470, 476 to 481, 508 to 513, 529 to 534, 540 to 545, 573 to 578, 601 to 606, 612 to 617, 623 to 628. More preferably wherein the first antibody comprises CDR sequences comprising any one or more of SEQ ID NO: 029, 136, 256, 372, 432 or 573, and the second antibody comprises CDR sequences comprising any one or more of SEQ ID NO: 651, 652, 653, 654, 655 or SEQ ID NO.: 656. Preferably wherein the composition is administered for prophylactic and/or therapeutic treatment.

In a preferred embodiment, a first antibody as disclosed herein is provided as a composition in combination with a second antibody, wherein the first antibody comprises a CDR sequence comprising any one of SEQ ID NO: 004 to 009, 015 to 020, 029 to 034, 039 to 044, 047 to 052, 065 to 070, 082 to 087, 103 to 108, 125 to 130, 136 to 141, 147 to 152, 191 to 196, 210 to 215, 221 to 226, 245 to 250, 256 to 261, 286 to 291, 297 to 302, 319 to 324, 330 to 335, 350 to 355, 372 to 377, 383 to 388, 415 to 420, 432 to 437, 443 to 448, 454 to 459, 465 to 470, 476 to 481, 508 to 513, 529 to 534, 540 to 545, 573 to 578, 601 to 606, 612 to 617, 623 to 628. More preferably wherein the first antibody comprises CDR sequences comprising any one or more of SEQ ID NO: 029, 136, 256, 372, 432 or 573, and the second antibody comprises CDR sequences comprising any one or more of SEQ ID NO: 657, 658, 659, 660, 661 or SEQ ID NO.: 662. Preferably wherein the composition is administered for prophylactic and/or therapeutic treatment.

Preferably, the composition of the invention is a water-based composition such as an aqueous liquid. Preferably, the composition of the invention further comprises one or more salts, for example sodium chloride. The composition of the invention may further comprise one or more buffering agents, for example sodium acetate. The composition of the invention may further comprise one or more carbohydrates, such as sucrose, or other active ingredients, such as other antibodies, neuraminidase inhibitors, endonuclease inhibitors, and adjuvants such as oils, cytokines, emulsifiers, or combinations thereof. The pH of the composition of the invention can be between 4 and 8, more preferably the pH is around 5.5. Preferably the pH is around 7.4.

### Mucosal Composition

The term ‘mucosal composition’, as used herein, refers to any pharmaceutical formulation that may be topically applied to a mucosal surface and delays or resists the mucosal flushing or removal action.

The term ‘mucosal composition’, may be used in reference to a route of administration in which an antibody, as disclosed herein, is provided into the nasal cavity, oropharyngeal region or upper respiratory tract, preferably through the nostrils, as part of a prophylactic and/or therapeutic treatment as disclosed herein.

### Mucosally

The mucosa comprises membranes that line cavities in the human body, covering internal organs, and consist of one or more layers of epithelial cells and loose connective tissue, which may produce mucus. The term ‘mucosally’, as used herein, may also be referred to as ‘mucosal administration’, and includes reference to a route of administration in which a drug is prophylactically and/or therapeutically provided to the mucosa, for example, mucosa found in the nose, mouth, lungs, vagina, rectum or stomach. The mucous membrane lubricates and protects these organs and cavities from abrasive particles and bodily fluids, as well as invasive pathogens.

Preferably, the antibodies of the invention are administered mucosally.

### Intranasally

The term ‘intranasally’, as used herein, may also be referred to as ‘nasal administration’, and includes reference to a route of administration in which a drug is provided into the upper respiratory tract and/or lower respiratory tract, preferably through the nostrils, as part of a prophylactic and/or therapeutic treatment as disclosed herein. Preferably, the administration provides for drug in the nasal cavity. The back section of the nasal cavity is also referred to as the pharynx. Nasal administration preferably provides for delivery of antibodies as disclosed herein in the mucous membrane lining the nasal cavity. Intranasal administration can be performed using, for instance, a nasal spray or nose drops. In some embodiments, the drug is delivered to the nasal cavity via the oral route. For example, RetroNose uses a breath-actuated pressurised metered-dose inhaler (pMDI) to administer drugs through the buccal cavity during the nasal expiratory phase. Such methods allow the drug particles to enter the nasal cavities through the pharynx.

Preferably, the antibodies of the invention are administered intranasally.

With intranasal administration, a drug is provided through the nostrils to the upper respiratory tract as part of a prophylactic and/or therapeutic treatment as disclosed herein. Preferably, the administration provides for drug in the nasal cavity. Nasal administration can either be a form of topical administration or systemic administration, as the drugs thus locally delivered can go on to have either local or systemic effects. In the present case, with antibodies as disclosed herein, nasal administration is preferably a form of topical administration.

Intranasal administration as disclosed herein may be done using a medicament in liquid form, preferably in the form of drops or nasal spray. The aqueous liquid may comprise adjuvants. These adjuvants may for example be salts, oils, cytokines, emulsifiers, buffering agents, carbohydrates and combinations thereof. Intranasal administration may also be done using a medicament in solid form, such as powders.

### Oral Inhalation

The term ‘oral inhalation’, as used herein, may also be referred to as ‘mouth inhalation’, and includes reference to a route of administration in which a drug is provided through the mouth to the upper and/or lower respiratory tract such as lungs, as part of a prophylactic and/or therapeutic treatment of the invention. Oral inhalation may for example be applied for drugs in their powdered form and drugs in the form of liquid droplets or aerosols.

Preferably, the antibodies of the invention are administered by oral inhalation.

With oral inhalation, a drug is provided through the mouth to the respiratory tract, preferably lower respiratory tract such as lungs, as part of a prophylactic and/or therapeutic treatment of the invention. As described further herein, oral inhalation also includes nasal drug delivery (also referred to as nasal drug delivery via the oral route).

Oral inhalation may for example be applied for drugs in their powdered form and drugs in the form of liquid droplets or aerosols. Oral inhalation may include the use of an inhaler. The inhaler may be involved in the achievement of the dose that was determined.

The drug that is administered by oral inhalation may reach the lung but may also partially be cleared out by exhalation.

Administration by oral inhalation as disclosed herein may be done using a medicament comprising aerosols in powdered (solid) or liquid form. Powdered aerosols comprising particles smaller than 3 μm in diameter will primarily reach the respiratory region of the lung, and will therefore be absorbed better than larger particles. The medicament may comprise adjuvants. These adjuvants may for example be salts, oils, cytokines, emulsifiers, buffering agents, carbohydrates and combinations thereof.

### Oropharyngeal Administration

The term ‘oropharyngeal administration’, as used herein, may also be referred to as delivery to the part of the pharynx that lies between the soft palate and the hyoid bone and includes reference to a route of administration in which a drug is provided via either the mouth or nasal passages as part of a prophylactic and/or therapeutic treatment.

Oropharyngeal administration may, for example, be used for drugs in their powdered form and drugs in the form of liquid droplets or aerosols.

Preferably, the antibodies of the invention are administered by oropharyngeal administration.

### Prior to

The term ‘prior to’, as used herein, includes reference to the administration of an antibody before an individual has been exposed to, or is infected with, a coronavirus.

Preferably, the antibody as disclosed is administered to an individual up to 24 hours prior to coronavirus exposure, for example between zero and 24 hours before the individual has been exposed to said coronavirus.

Preferably, the antibody as disclosed is administered to an individual up to 48 hours prior to coronavirus exposure, for example between zero and 48 hours before the individual has been exposed to said coronavirus.

In a preferred embodiment, the antibody is administered 2 days or more prior to coronavirus exposure. In a preferred embodiment, the antibody is administered 3 days or more prior to coronavirus exposure. In a preferred embodiment, the antibody is administered 4 days, or more prior to coronavirus exposure. In a preferred embodiment, the antibody is administered 5 days or more prior to coronavirus exposure. In a preferred embodiment, the antibody is administered 6 days or more prior to coronavirus exposure. In a preferred embodiment, the antibody is administered 7 days or more prior to coronavirus exposure.

### Dosage

The term ‘dosage’ as used herein, refers to the amount of antibody to be given at a particular time (e.g., over the course of a 24-hour, 12-hour, 30-minute period, etc.). A dose refers to a single dosing episode, whether the dose is a unit dosage form or multiple unit dosage forms taken together (e.g., ingestion of two or more pills, receiving two or more nasal administrations). A dosage includes reference to a pharmaceutical dosage form wherein the medicament is packaged for administration as, e.g., a single-unit dose or multiple-unit dose. A dosage may also be administered as, e.g., one or more drops of an antibody-comprising composition (e.g., nasal drops) or one or more sprays of an antibody-comprising composition (e.g., nasal sprays).

Preferably, a suitable dosage of an antibody as disclosed herein contains a dose of between 0.01 mg and 20 mg, preferably 0.1 mg and 15 mg, more preferably around 0.5 mg and 10 mg or even around 1 mg, e.g. when the dosage is for intranasal administration. Such dosages are also referred to as “flat dosages” or “nominal dosages” in contrast to dosages based on the weight of the patient. Flat dosages have the advantage that the medicament can be packaged in a single-unit dose, for example. A single dose of an antibody according to the present invention can provide protection from coronavirus infection for several days and may be provided “on demand” or “as needed”. For example, an individual may administer antibody before leaving the house or before coming into contact with other individuals.

In order to provide long-lasting protection, the antibody may be administered on a regular basis. For example, the antibody is administered once, or at least once per month. In a preferred embodiment, the antibody is administered once, or at least once per week, e.g., twice weekly. In a preferred embodiment, the antibody is administered once, or at least once per day. As is clear to a skilled person, less antibody may be administered when the antibody is administered more frequently (e.g., daily). In a preferred embodiment, between 0.01 mg to 20 mg of antibody is administered per week (e.g., once or twice weekly or daily). In an exemplary embodiment, between 0.1 mg and 25 mg is administered daily (i.e. between 0.7 mg and 175 mg per week). In an exemplary embodiment, 0.5 mg to 3.5 mg is administered daily (i.e. between 3.5 mg and 24.5 mg per week).

The invention also provides a composition formulated for intranasal administration and/or oral inhalation comprising an antibody as disclosed herein, preferably in a single dose unit between 0.1 mg to 20 mg, preferably 0.5 mg to 15 mg or preferably 1 mg to 12.5 mg.

### Use in a Method for Treatment

The present invention relates inter alia to an antibody as disclosed herein, for use in a method for treatment of coronavirus infection in an individual, more specifically the antibody can be used in a method for the prophylactic and/or therapeutic treatment of coronavirus infection in an individual.

### Complementarity-Determining Regions (CDRs)

Preferably, the CDR regions are identified according to Kabat et al. (1991) as described in Sequences of Proteins of Immunological Interest.

In a preferred embodiment, the binding interaction of the binding molecules, preferably the antibody, and the stem helix is mediated exclusively by light and heavy chain variable sequences.

The antibody as disclosed herein may be capable of specifically binding to a coronavirus that is in attenuated or inactivated form or that is viable, and/or in an infective form.

The antibody as disclosed herein is also capable of specifically binding to one or more fragments of the coronavirus.

An antibody as disclosed herein comprises a preferred heavy chain CDR1 sequence comprising any one of the following SEQ ID NO: 004 to 009, 015 to 020, 029 to 034, 039 to 044, 047 to 052, 065 to 070. A CDR comprising the amino acid sequence SEQ ID NO: 029 is especially preferred.

An antibody as disclosed herein comprises a preferred heavy chain CDR2 sequence comprising any one of the following SEQ ID NO: 082 to 087, 103 to 108, 125 to 130, 136 to 141, 147 to 152, 191 to 196. A CDR comprising the amino acid sequence SEQ ID NO: 136 is especially preferred.

An antibody as disclosed herein comprises a preferred heavy chain CDR3 sequence comprising any one of the following SEQ ID NO: 210 to 215, 221 to 226, 245 to 250, 256 to 261, 286 to 291, 297 to 302. A CDR comprising the amino acid sequence SEQ ID NO: 256 is especially preferred.

An antibody as disclosed herein comprises a preferred light chain CDR1 sequence comprising any one of the following SEQ ID NO: 319 to 324, 330 to 335, 350 to 355, 372 to 377, 383 to 388, 415 to 420. A CDR comprising the amino acid sequence SEQ ID NO: 372 is especially preferred.

An antibody as disclosed herein comprises a preferred light chain CDR2 sequence comprising any one of the following SEQ ID NO: 432 to 437, 443 to 448, 454 to 459, 465 to 470, 476 to 481, 508 to 513. A CDR comprising the amino acid sequence SEQ ID NO: 432 is especially preferred.

An antibody as disclosed herein comprises a preferred light chain CDR3 sequence comprising any one of the following SEQ ID NO: 529 to 534, 540 to 545, 573 to 578, 601 to 606, 612 to 617, 623 to 628. A CDR comprising the amino acid sequence SEQ ID NO: 573 is especially preferred.

An antibody as disclosed herein comprises a heavy chain variable domain that further comprises at least a heavy chain framework region FR1 of SEQ ID NO: 631.

An antibody as disclosed herein comprises a heavy chain variable domain that further comprises at least a heavy chain framework region FR2 of SEQ ID NO: 632.

An antibody as disclosed herein comprises a heavy chain variable domain that further comprises at least a heavy chain framework region FR3 of SEQ ID NO: 633.

An antibody as disclosed herein comprises a heavy chain variable domain that further comprises at least a heavy chain framework region FR4 of SEQ ID NO: 634.

In a preferred embodiment, the heavy chain variable domain of said antibody comprises—a heavy chain framework region FR1 of SEQ ID NO: 631, a heavy chain framework region FR2 of SEQ ID NO: 632, a heavy chain framework region FR3 of SEQ ID NO: 633, and/or a heavy chain framework region FR4 of SEQ ID NO: 634, preferably all said heavy chain heavy chain framework regions FR1 to FR4.

An antibody as disclosed herein comprises a heavy chain variable domain that further comprises at least a light chain framework region FR1 of SEQ ID NO: 635.

An antibody as disclosed herein comprises a heavy chain variable domain that further comprises at least a light chain framework region FR2 of SEQ ID NO: 636.

An antibody as disclosed herein comprises a heavy chain variable domain that further comprises at least a light chain framework region FR3 of SEQ ID NO: 637.

An antibody as disclosed herein comprises a heavy chain variable domain that further comprises at least a light chain framework region FR4 of SEQ ID NO: 638.

In a preferred embodiment, the light chain variable domain of said antibody comprises—a light chain framework region FR1 of SEQ ID NO: 635, a light chain framework region FR2 of SEQ ID NO: 636, a light chain framework region FR3 of SEQ ID NO: 637, and/or a light chain framework region FR4 of SEQ ID NO: 638, preferably all said light chain framework regions are FR1 to FR4.

Most preferably, an antibody as disclosed herein comprises a heavy chain variable domain having the sequence of SEQ ID NO: 629 and/or a light chain variable domain having the sequence of SEQ ID NO: 630.

In treatment methods of the invention, the heavy chain variable domain (VH) of the antibody is preferably indicated as SEQ ID NO:629. In treatment methods or compositions of the invention, the light chain variable domain (VL) of the antibody is preferably indicated as SEQ ID NO:630. In treatment methods or compositions of the invention, the antibody can be an antibody as disclosed herein, or a binding molecule comprising an antigen-binding domain such as a variable domain (V) thereof.

Preferably, an antibody as disclosed herein comprises a heavy chain variable domain having the sequence of SEQ ID NO:629 having at most 15, preferably 0, 1, 2, 3, 4, 5, 6, 7, 8, 9 or 10, more preferably having 0, 1, 2, 3, 4 or 5 amino acid insertions, deletions, substitutions.

Preferably, an antibody as disclosed herein comprises a light chain variable domain having the sequence of SEQ ID NO:630 having at most 15, preferably 0, 1, 2, 3, 4, 5, 6, 7, 8, 9 or 10, more preferably having 0, 1, 2, 3, 4 or 5 amino acid insertions, deletions, substitutions.

Preferably, said amino acid insertions, deletions, substitutions in respect of either the heavy chain variable domain and/or the light chain variable domain are not in the CDRs.

### SEQUENCES

CDRs and framework regions defined herein are based on the Kabat numbering scheme as described in Sequences of Proteins of Immunological Interest (Kabat et al., 1991).

Heavy chain CDR1 region
(SEQ ID NO: 001 to SEQ ID NO: 076)

| | |
|---|---|
| SEQ ID NO.: 001 | FTSY |
| SEQ ID NO.: 002 | FTSYY |
| SEQ ID NO.: 003 | FTSYYM |
| SEQ ID NO.: 004 | FTSYYMH |
| SEQ ID NO.: 005 | FTSYYMHW |
| SEQ ID NO.: 006 | FTSYYMHWV |
| SEQ ID NO.: 007 | FTSYYMHWVR |
| SEQ ID NO.: 008 | FTSYYMHWVRQ |
| SEQ ID NO.: 009 | FTSYYMHWVRQA |
| SEQ ID NO.: 010 | GYTFT |
| SEQ ID NO.: 011 | GYTFTS |
| SEQ ID NO.: 012 | GYTFTSY |
| SEQ ID NO.: 013 | GYTFTSYY |
| SEQ ID NO.: 014 | GYTFTSYYM |
| SEQ ID NO.: 015 | GYTFTSYYMH |
| SEQ ID NO.: 016 | GYTFTSYYMHW |
| SEQ ID NO.: 017 | GYTFTSYYMHWV |
| SEQ ID NO.: 018 | GYTFTSYYMHWVR |
| SEQ ID NO.: 019 | GYTFTSYYMHWVRQ |
| SEQ ID NO.: 020 | GYTFTSYYMHWVRQA |
| SEQ ID NO.: 021 | HWVR |
| SEQ ID NO.: 022 | HWVRQ |
| SEQ ID NO.: 023 | HWVRQA |
| SEQ ID NO.: 024 | MHWV |
| SEQ ID NO.: 025 | MHWVR |
| SEQ ID NO.: 026 | MHWVRQ |
| SEQ ID NO.: 027 | MHWVRQA |
| SEQ ID NO.: 028 | SYYM |
| SEQ ID NO.: 029 | SYYMH |
| SEQ ID NO.: 030 | SYYMHW |
| SEQ ID NO.: 031 | SYYMHWV |
| SEQ ID NO.: 032 | SYYMHWVR |
| SEQ ID NO.: 033 | SYYMHWVRQ |
| SEQ ID NO.: 034 | SYYMHWVRQA |
| SEQ ID NO.: 035 | TFTS |
| SEQ ID NO.: 036 | TFTSY |
| SEQ ID NO.: 037 | TFTSYY |
| SEQ ID NO.: 038 | TFTSYYM |
| SEQ ID NO.: 039 | TFTSYYMH |
| SEQ ID NO.: 040 | TFTSYYMHW |
| SEQ ID NO.: 041 | TFTSYYMHWV |
| SEQ ID NO.: 042 | TFTSYYMHWVR |
| SEQ ID NO.: 043 | TFTSYYMHWVRQ |
| SEQ ID NO.: 044 | TFTSYYMHWVRQA |
| SEQ ID NO.: 045 | TSYY |
| SEQ ID NO.: 046 | TSYYM |
| SEQ ID NO.: 047 | TSYYMH |
| SEQ ID NO.: 048 | TSYYMHW |
| SEQ ID NO.: 049 | TSYYMHWV |
| SEQ ID NO.: 050 | TSYYMHWVR |
| SEQ ID NO.: 051 | TSYYMHWVRQ |
| SEQ ID NO.: 052 | TSYYMHWVRQA |
| SEQ ID NO.: 053 | WVRQ |
| SEQ ID NO.: 054 | WVRQA |
| SEQ ID NO.: 055 | YMHW |
| SEQ ID NO.: 056 | YMHWV |
| SEQ ID NO.: 057 | YMHWVR |
| SEQ ID NO.: 058 | YMHWVRQ |
| SEQ ID NO.: 059 | YMHWVRQA |
| SEQ ID NO.: 060 | YTFT |
| SEQ ID NO.: 061 | YTFTS |
| SEQ ID NO.: 062 | YTFTSY |
| SEQ ID NO.: 063 | YTFTSYY |
| SEQ ID NO.: 064 | YTFTSYYM |
| SEQ ID NO.: 065 | YTFTSYYMH |
| SEQ ID NO.: 066 | YTFTSYYMHW |
| SEQ ID NO.: 067 | YTFTSYYMHWV |
| SEQ ID NO.: 068 | YTFTSYYMHWVR |
| SEQ ID NO.: 069 | YTFTSYYMHWVRQ |
| SEQ ID NO.: 070 | YTFTSYYMHWVRQA |
| SEQ ID NO.: 071 | YYMH |
| SEQ ID NO.: 072 | YYMHW |
| SEQ ID NO.: 073 | YYMHWV |
| SEQ ID NO.: 074 | YYMHWVR |
| SEQ ID NO.: 075 | YYMHWVRQ |
| SEQ ID NO.: 076 | YYMHWVRQA |

Heavy chain CDR2 region
(SEQ ID NO: 077 to SEQ ID NO: 196)

| | |
|---|---|
| SEQ ID NO.: 077 | EWMGLITPSGDDTYYA |
| SEQ ID NO.: 078 | EWMGLITPSGDDTYYAQ |
| SEQ ID NO.: 079 | EWMGLITPSGDDTYYAQR |
| SEQ ID NO.: 080 | EWMGLITPSGDDTYYAQRF |
| SEQ ID NO.: 081 | EWMGLITPSGDDTYYAQRFQ |
| SEQ ID NO.: 082 | EWMGLITPSGDDTYYAQRFQG |
| SEQ ID NO.: 083 | EWMGLITPSGDDTYYAQRFQGR |
| SEQ ID NO.: 084 | EWMGLITPSGDDTYYAQRFQGRV |
| SEQ ID NO.: 085 | EWMGLITPSGDDTYYAQRFQGRVT |
| SEQ ID NO.: 086 | EWMGLITPSGDDTYYAQRFQGRVTM |
| SEQ ID NO.: 087 | EWMGLITPSGDDTYYAQRFQGRVTMT |
| SEQ ID NO.: 088 | GDDTYYAQ |
| SEQ ID NO.: 089 | GDDTYYAQR |
| SEQ ID NO.: 090 | GDDTYYAQRF |
| SEQ ID NO.: 091 | GDDTYYAQRFQ |
| SEQ ID NO.: 092 | GDDTYYAQRFQG |
| SEQ ID NO.: 093 | GDDTYYAQRFQGR |
| SEQ ID NO.: 094 | GDDTYYAQRFQGRV |
| SEQ ID NO.: 095 | GDDTYYAQRFQGRVT |
| SEQ ID NO.: 096 | GDDTYYAQRFQGRVTM |
| SEQ ID NO.: 097 | GDDTYYAQRFQGRVTMT |
| SEQ ID NO.: 098 | GLITPSGDDTYYA |
| SEQ ID NO.: 099 | GLITPSGDDTYYAQ |
| SEQ ID NO.: 100 | GLITPSGDDTYYAQR |
| SEQ ID NO.: 101 | GLITPSGDDTYYAQRF |
| SEQ ID NO.: 102 | GLITPSGDDTYYAQRFQ |
| SEQ ID NO.: 103 | GLITPSGDDTYYAQRFQG |
| SEQ ID NO.: 104 | GLITPSGDDTYYAQRFQGR |
| SEQ ID NO.: 105 | GLITPSGDDTYYAQRFQGRV |
| SEQ ID NO.: 106 | GLITPSGDDTYYAQRFQGRVT |
| SEQ ID NO.: 107 | GLITPSGDDTYYAQRFQGRVTM |
| SEQ ID NO.: 108 | GLITPSGDDTYYAQRFQGRVTMT |
| SEQ ID NO.: 109 | ITPSGDDTYYA |
| SEQ ID NO.: 110 | ITPSGDDTYYAQ |
| SEQ ID NO.: 111 | ITPSGDDTYYAQR |
| SEQ ID NO.: 112 | ITPSGDDTYYAQRF |
| SEQ ID NO.: 113 | ITPSGDDTYYAQRFQ |
| SEQ ID NO.: 114 | ITPSGDDTYYAQRFQG |
| SEQ ID NO.: 115 | ITPSGDDTYYAQRFQGR |
| SEQ ID NO.: 116 | ITPSGDDTYYAQRFQGRV |
| SEQ ID NO.: 117 | ITPSGDDTYYAQRFQGRVT |
| SEQ ID NO.: 118 | ITPSGDDTYYAQRFQGRVTM |
| SEQ ID NO.: 119 | ITPSGDDTYYAQRFQGRVTMT |
| SEQ ID NO.: 120 | LEWMGLITPSGDDTYYA |
| SEQ ID NO.: 121 | LEWMGLITPSGDDTYYAQ |
| SEQ ID NO.: 122 | LEWMGLITPSGDDTYYAQR |
| SEQ ID NO.: 123 | LEWMGLITPSGDDTYYAQRF |
| SEQ ID NO.: 124 | LEWMGLITPSGDDTYYAQRFQ |
| SEQ ID NO.: 125 | LEWMGLITPSGDDTYYAQRFQG |
| SEQ ID NO.: 126 | LEWMGLITPSGDDTYYAQRFQGR |
| SEQ ID NO.: 127 | LEWMGLITPSGDDTYYAQRFQGRV |
| SEQ ID NO.: 128 | LEWMGLITPSGDDTYYAQRFQGRVT |
| SEQ ID NO.: 129 | LEWMGLITPSGDDTYYAQRFQGRVTM |
| SEQ ID NO.: 130 | LEWMGLITPSGDDTYYAQRFQGRVTMT |
| SEQ ID NO.: 131 | LITPSGDDTYYA |
| SEQ ID NO.: 132 | LITPSGDDTYYAQ |
| SEQ ID NO.: 133 | LITPSGDDTYYAQR |
| SEQ ID NO.: 134 | LITPSGDDTYYAQRF |
| SEQ ID NO.: 135 | LITPSGDDTYYAQRFQ |
| SEQ ID NO.: 136 | LITPSGDDTYYAQRFQG |
| SEQ ID NO.: 137 | LITPSGDDTYYAQRFQGR |
| SEQ ID NO.: 138 | LITPSGDDTYYAQRFQGRV |
| SEQ ID NO.: 139 | LITPSGDDTYYAQRFQGRVT |
| SEQ ID NO.: 140 | LITPSGDDTYYAQRFQGRVTM |
| SEQ ID NO.: 141 | LITPSGDDTYYAQRFQGRVTMT |
| SEQ ID NO.: 142 | MGLITPSGDDTYYA |
| SEQ ID NO.: 143 | MGLITPSGDDTYYAQ |
| SEQ ID NO.: 144 | MGLITPSGDDTYYAQR |
| SEQ ID NO.: 145 | MGLITPSGDDTYYAQRF |
| SEQ ID NO.: 146 | MGLITPSGDDTYYAQRFQ |
| SEQ ID NO.: 147 | MGLITPSGDDTYYAQRFQG |
| SEQ ID NO.: 148 | MGLITPSGDDTYYAQRFQGR |
| SEQ ID NO.: 149 | MGLITPSGDDTYYAQRFQGRV |
| SEQ ID NO.: 150 | MGLITPSGDDTYYAQRFQGRVT |
| SEQ ID NO.: 151 | MGLITPSGDDTYYAQRFQGRVTM |
| SEQ ID NO.: 152 | MGLITPSGDDTYYAQRFQGRVTMT |
| SEQ ID NO.: 153 | PSGDDTYYA |
| SEQ ID NO.: 154 | PSGDDTYYAQ |
| SEQ ID NO.: 155 | PSGDDTYYAQR |
| SEQ ID NO.: 156 | PSGDDTYYAQRF |
| SEQ ID NO.: 157 | PSGDDTYYAQRFQ |
| SEQ ID NO.: 158 | PSGDDTYYAQRFQG |
| SEQ ID NO.: 159 | PSGDDTYYAQRFQGR |
| SEQ ID NO.: 160 | PSGDDTYYAQRFQGRV |
| SEQ ID NO.: 161 | PSGDDTYYAQRFQGRVT |
| SEQ ID NO.: 162 | PSGDDTYYAQRFQGRVTM |
| SEQ ID NO.: 163 | PSGDDTYYAQRFQGRVTMT |
| SEQ ID NO.: 164 | SGDDTYYA |
| SEQ ID NO.: 165 | SGDDTYYAQ |
| SEQ ID NO.: 166 | SGDDTYYAQR |
| SEQ ID NO.: 167 | SGDDTYYAQRF |
| SEQ ID NO.: 168 | SGDDTYYAQRFQ |
| SEQ ID NO.: 169 | SGDDTYYAQRFQG |
| SEQ ID NO.: 170 | SGDDTYYAQRFQGR |
| SEQ ID NO.: 171 | SGDDTYYAQRFQGRV |
| SEQ ID NO.: 172 | SGDDTYYAQRFQGRVT |
| SEQ ID NO.: 173 | SGDDTYYAQRFQGRVTM |
| SEQ ID NO.: 174 | SGDDTYYAQRFQGRVTMT |
| SEQ ID NO.: 175 | TPSGDDTYYA |
| SEQ ID NO.: 176 | TPSGDDTYYAQ |
| SEQ ID NO.: 177 | TPSGDDTYYAQR |
| SEQ ID NO.: 178 | TPSGDDTYYAQRF |
| SEQ ID NO.: 179 | TPSGDDTYYAQRFQ |
| SEQ ID NO.: 180 | TPSGDDTYYAQRFQG |
| SEQ ID NO.: 181 | TPSGDDTYYAQRFQGR |
| SEQ ID NO.: 182 | TPSGDDTYYAQRFQGRV |
| SEQ ID NO.: 183 | TPSGDDTYYAQRFQGRVT |
| SEQ ID NO.: 184 | TPSGDDTYYAQRFQGRVTM |
| SEQ ID NO.: 185 | TPSGDDTYYAQRFQGRVTMT |
| SEQ ID NO.: 186 | WMGLITPSGDDTYYA |
| SEQ ID NO.: 187 | WMGLITPSGDDTYYAQ |
| SEQ ID NO.: 188 | WMGLITPSGDDTYYAQR |
| SEQ ID NO.: 189 | WMGLITPSGDDTYYAQRF |
| SEQ ID NO.: 190 | WMGLITPSGDDTYYAQRFQ |
| SEQ ID NO.: 191 | WMGLITPSGDDTYYAQRFQG |
| SEQ ID NO.: 192 | WMGLITPSGDDTYYAQRFQGR |
| SEQ ID NO.: 193 | WMGLITPSGDDTYYAQRFQGRV |
| SEQ ID NO.: 194 | WMGLITPSGDDTYYAQRFQGRVT |
| SEQ ID NO.: 195 | WMGLITPSGDDTYYAQRFQGRVTM |
| SEQ ID NO.: 196 | WMGLITPSGDDTYYAQRFQGRVTMT |

Heavy chain CDR3 region
(SEQ ID NO: 197 to SEQ ID NO: 302)

| | |
|---|---|
| SEQ ID NO.: 197 | AGGF |
| SEQ ID NO.: 198 | AGGFD |
| SEQ ID NO.: 199 | AGGFDV |
| SEQ ID NO.: 200 | AGGFDVW |
| SEQ ID NO.: 201 | AGGFDVWG |
| SEQ ID NO.: 202 | AGGFDVWGQ |
| SEQ ID NO.: 203 | AGGFDVWGQG |
| SEQ ID NO.: 204 | AGGFDVWGQGT |
| SEQ ID NO.: 205 | AKMSRA |
| SEQ ID NO.: 206 | AKMSRAG |
| SEQ ID NO.: 207 | AKMSRAGG |
| SEQ ID NO.: 208 | AKMSRAGGF |
| SEQ ID NO.: 209 | AKMSRAGGED |
| SEQ ID NO.: 210 | AKMSRAGGFDV |
| SEQ ID NO.: 211 | AKMSRAGGFDVW |
| SEQ ID NO.: 212 | AKMSRAGGFDVWG |
| SEQ ID NO.: 213 | AKMSRAGGFDVWGQ |
| SEQ ID NO.: 214 | AKMSRAGGFDVWGQG |
| SEQ ID NO.: 215 | AKMSRAGGFDVWGQGT |
| SEQ ID NO.: 216 | CAKMSRA |
| SEQ ID NO.: 217 | CAKMSRAG |
| SEQ ID NO.: 218 | CAKMSRAGG |
| SEQ ID NO.: 219 | CAKMSRAGGF |
| SEQ ID NO.: 220 | CAKMSRAGGED |
| SEQ ID NO.: 221 | CAKMSRAGGFDV |
| SEQ ID NO.: 222 | CAKMSRAGGEDVW |
| SEQ ID NO.: 223 | CAKMSRAGGFDVWG |
| SEQ ID NO.: 224 | CAKMSRAGGFDVWGQ |
| SEQ ID NO.: 225 | CAKMSRAGGFDVWGQG |
| SEQ ID NO.: 226 | CAKMSRAGGFDVWGQGT |
| SEQ ID NO.: 227 | GFDV |
| SEQ ID NO.: 228 | GFDVW |
| SEQ ID NO.: 229 | GFDVWG |
| SEQ ID NO.: 230 | GFDVWGQ |
| SEQ ID NO.: 231 | GFDVWGQG |
| SEQ ID NO.: 232 | GFDVWGQGT |
| SEQ ID NO.: 233 | GGFD |
| SEQ ID NO.: 234 | GGFDV |
| SEQ ID NO.: 235 | GGFDVW |
| SEQ ID NO.: 236 | GGFDVWG |
| SEQ ID NO.: 237 | GGFDVWGQ |
| SEQ ID NO.: 238 | GGFDVWGQG |
| SEQ ID NO.: 239 | GGFDVWGQGT |
| SEQ ID NO.: 240 | KMSRA |
| SEQ ID NO.: 241 | KMSRAG |
| SEQ ID NO.: 242 | KMSRAGG |
| SEQ ID NO.: 243 | KMSRAGGF |
| SEQ ID NO.: 244 | KMSRAGGED |
| SEQ ID NO.: 245 | KMSRAGGFDV |
| SEQ ID NO.: 246 | KMSRAGGFDVW |
| SEQ ID NO.: 247 | KMSRAGGFDVWG |
| SEQ ID NO.: 248 | KMSRAGGFDVWGQ |
| SEQ ID NO.: 249 | KMSRAGGFDVWGQG |
| SEQ ID NO.: 250 | KMSRAGGFDVWGQGT |
| SEQ ID NO.: 251 | MSRA |
| SEQ ID NO.: 252 | MSRAG |
| SEQ ID NO.: 253 | MSRAGG |
| SEQ ID NO.: 254 | MSRAGGF |
| SEQ ID NO.: 255 | MSRAGGED |
| SEQ ID NO.: 256 | MSRAGGFDV |
| SEQ ID NO.: 257 | MSRAGGFDVW |
| SEQ ID NO.: 258 | MSRAGGFDVWG |
| SEQ ID NO.: 259 | MSRAGGFDVWGQ |
| SEQ ID NO.: 260 | MSRAGGFDVWGQG |
| SEQ ID NO.: 261 | MSRAGGFDVWGQGT |
| SEQ ID NO.: 262 | RAGG |
| SEQ ID NO.: 263 | RAGGF |
| SEQ ID NO.: 264 | RAGGED |
| SEQ ID NO.: 265 | RAGGFDV |
| SEQ ID NO.: 266 | RAGGFDVW |
| SEQ ID NO.: 267 | RAGGFDVWG |
| SEQ ID NO.: 268 | RAGGFDVWGQ |
| SEQ ID NO.: 269 | RAGGFDVWGQG |
| SEQ ID NO.: 270 | RAGGFDVWGQGT |
| SEQ ID NO.: 271 | SRAG |
| SEQ ID NO.: 272 | SRAGG |
| SEQ ID NO.: 273 | SRAGGF |
| SEQ ID NO.: 274 | SRAGGFD |
| SEQ ID NO.: 275 | SRAGGFDV |
| SEQ ID NO.: 276 | SRAGGFDVW |
| SEQ ID NO.: 277 | SRAGGFDVWG |
| SEQ ID NO.: 278 | SRAGGFDVWGQ |
| SEQ ID NO.: 279 | SRAGGFDVWGQG |
| SEQ ID NO.: 280 | SRAGGFDVWGQGT |
| SEQ ID NO.: 281 | YCAKMSRA |
| SEQ ID NO.: 282 | YCAKMSRAG |
| SEQ ID NO.: 283 | YCAKMSRAGG |
| SEQ ID NO.: 284 | YCAKMSRAGGF |
| SEQ ID NO.: 285 | YCAKMSRAGGED |
| SEQ ID NO.: 286 | YCAKMSRAGGFDV |
| SEQ ID NO.: 287 | YCAKMSRAGGFDVW |
| SEQ ID NO.: 288 | YCAKMSRAGGFDVWG |
| SEQ ID NO.: 289 | YCAKMSRAGGFDVWGQ |
| SEQ ID NO.: 290 | YCAKMSRAGGFDVWGQG |
| SEQ ID NO.: 291 | YCAKMSRAGGFDVWGQGT |
| SEQ ID NO.: 292 | YYCAKMSRA |
| SEQ ID NO.: 293 | YYCAKMSRAG |
| SEQ ID NO.: 294 | YYCAKMSRAGG |
| SEQ ID NO.: 295 | YYCAKMSRAGGF |
| SEQ ID NO.: 296 | YYCAKMSRAGGED |
| SEQ ID NO.: 297 | YYCAKMSRAGGFDV |
| SEQ ID NO.: 298 | YYCAKMSRAGGFDVW |
| SEQ ID NO.: 299 | YYCAKMSRAGGFDVWG |
| SEQ ID NO.: 300 | YYCAKMSRAGGFDVWGQ |
| SEQ ID NO.: 301 | YYCAKMSRAGGFDVWGQG |
| SEQ ID NO.: 302 | YYCAKMSRAGGFDVWGQGT |

Light chain CDR1 region
(SEQ ID NO: 303 to SEQ ID NO: 420)

| | |
|---|---|
| SEQ ID NO.: 303 | ASQSIT |
| SEQ ID NO.: 304 | ASQSITG |
| SEQ ID NO.: 305 | ASQSITGR |
| SEQ ID NO.: 306 | ASQSITGRY |
| SEQ ID NO.: 307 | ASQSITGRYL |
| SEQ ID NO.: 308 | ASQSITGRYLA |
| SEQ ID NO.: 309 | ASQSITGRYLAW |
| SEQ ID NO.: 310 | ASQSITGRYLAWY |
| SEQ ID NO.: 311 | ASQSITGRYLAWYQ |
| SEQ ID NO.: 312 | ASQSITGRYLAWYQQ |
| SEQ ID NO.: 313 | ASQSITGRYLAWYQQK |
| SEQ ID NO.: 314 | ATLSCRASQSIT |
| SEQ ID NO.: 315 | ATLSCRASQSITG |
| SEQ ID NO.: 316 | ATLSCRASQSITGR |
| SEQ ID NO.: 317 | ATLSCRASQSITGRY |
| SEQ ID NO.: 318 | ATLSCRASQSITGRYL |
| SEQ ID NO.: 319 | ATLSCRASQSITGRYLA |
| SEQ ID NO.: 320 | ATLSCRASQSITGRYLAW |
| SEQ ID NO.: 321 | ATLSCRASQSITGRYLAWY |
| SEQ ID NO.: 322 | ATLSCRASQSITGRYLAWYQ |
| SEQ ID NO.: 323 | ATLSCRASQSITGRYLAWYQQ |
| SEQ ID NO.: 324 | ATLSCRASQSITGRYLAWYQQK |
| SEQ ID NO.: 325 | CRASQSIT |
| SEQ ID NO.: 326 | CRASQSITG |
| SEQ ID NO.: 327 | CRASQSITGR |
| SEQ ID NO.: 328 | CRASQSITGRY |
| SEQ ID NO.: 329 | CRASQSITGRYL |
| SEQ ID NO.: 330 | CRASQSITGRYLA |
| SEQ ID NO.: 331 | CRASQSITGRYLAW |
| SEQ ID NO.: 332 | CRASQSITGRYLAWY |
| SEQ ID NO.: 333 | CRASQSITGRYLAWYQ |
| SEQ ID NO.: 334 | CRASQSITGRYLAWYQQ |
| SEQ ID NO.: 335 | CRASQSITGRYLAWYQQK |
| SEQ ID NO.: 336 | ITGR |
| SEQ ID NO.: 337 | ITGRY |
| SEQ ID NO.: 338 | ITGRYL |
| SEQ ID NO.: 339 | ITGRYLA |
| SEQ ID NO.: 340 | ITGRYLAW |
| SEQ ID NO.: 341 | ITGRYLAWY |
| SEQ ID NO.: 342 | ITGRYLAWYQ |
| SEQ ID NO.: 343 | ITGRYLAWYQQ |
| SEQ ID NO.: 344 | ITGRYLAWYQQK |
| SEQ ID NO.: 345 | LSCRASQSIT |
| SEQ ID NO.: 346 | LSCRASQSITG |
| SEQ ID NO.: 347 | LSCRASQSITGR |
| SEQ ID NO.: 348 | LSCRASQSITGRY |
| SEQ ID NO.: 349 | LSCRASQSITGRYL |
| SEQ ID NO.: 350 | LSCRASQSITGRYLA |
| SEQ ID NO.: 351 | LSCRASQSITGRYLAW |
| SEQ ID NO.: 352 | LSCRASQSITGRYLAWY |
| SEQ ID NO.: 353 | LSCRASQSITGRYLAWYQ |
| SEQ ID NO.: 354 | LSCRASQSITGRYLAWYQQ |
| SEQ ID NO.: 355 | LSCRASQSITGRYLAWYQQK |
| SEQ ID NO.: 356 | QSIT |
| SEQ ID NO.: 357 | QSITG |
| SEQ ID NO.: 358 | QSITGR |
| SEQ ID NO.: 359 | QSITGRY |
| SEQ ID NO.: 360 | QSITGRYL |
| SEQ ID NO.: 361 | QSITGRYLA |
| SEQ ID NO.: 362 | QSITGRYLAW |
| SEQ ID NO.: 363 | QSITGRYLAWY |
| SEQ ID NO.: 364 | QSITGRYLAWYQ |
| SEQ ID NO.: 365 | QSITGRYLAWYQQ |
| SEQ ID NO.: 366 | QSITGRYLAWYQQK |
| SEQ ID NO.: 367 | RASQSIT |
| SEQ ID NO.: 368 | RASQSITG |
| SEQ ID NO.: 369 | RASQSITGR |
| SEQ ID NO.: 370 | RASQSITGRY |
| SEQ ID NO.: 371 | RASQSITGRYL |
| SEQ ID NO.: 372 | RASQSITGRYLA |
| SEQ ID NO.: 373 | RASQSITGRYLAW |
| SEQ ID NO.: 374 | RASQSITGRYLAWY |
| SEQ ID NO.: 375 | RASQSITGRYLAWYQ |
| SEQ ID NO.: 376 | RASQSITGRYLAWYQQ |
| SEQ ID NO.: 377 | RASQSITGRYLAWYQQK |
| SEQ ID NO.: 378 | SCRASQSIT |
| SEQ ID NO.: 379 | SCRASQSITG |
| SEQ ID NO.: 380 | SCRASQSITGR |
| SEQ ID NO.: 381 | SCRASQSITGRY |
| SEQ ID NO.: 382 | SCRASQSITGRYL |
| SEQ ID NO.: 383 | SCRASQSITGRYLA |
| SEQ ID NO.: 384 | SCRASQSITGRYLAW |
| SEQ ID NO.: 385 | SCRASQSITGRYLAWY |
| SEQ ID NO.: 386 | SCRASQSITGRYLAWYQ |
| SEQ ID NO.: 387 | SCRASQSITGRYLAWYQQ |
| SEQ ID NO.: 388 | SCRASQSITGRYLAWYQQK |
| SEQ ID NO.: 389 | SITG |
| SEQ ID NO.: 390 | SITGR |
| SEQ ID NO.: 391 | SITGRY |
| SEQ ID NO.: 392 | SITGRYL |
| SEQ ID NO.: 393 | SITGRYLA |
| SEQ ID NO.: 394 | SITGRYLAW |
| SEQ ID NO.: 395 | SITGRYLAWY |
| SEQ ID NO.: 396 | SITGRYLAWYQ |
| SEQ ID NO.: 397 | SITGRYLAWYQQ |
| SEQ ID NO.: 398 | SITGRYLAWYQQK |
| SEQ ID NO.: 399 | SQSIT |
| SEQ ID NO.: 400 | SQSITG |
| SEQ ID NO.: 401 | SQSITGR |
| SEQ ID NO.: 402 | SQSITGRY |
| SEQ ID NO.: 403 | SQSITGRYL |
| SEQ ID NO.: 404 | SQSITGRYLA |
| SEQ ID NO.: 405 | SQSITGRYLAW |
| SEQ ID NO.: 406 | SQSITGRYLAWY |
| SEQ ID NO.: 407 | SQSITGRYLAWYQ |
| SEQ ID NO.: 408 | SQSITGRYLAWYQQ |
| SEQ ID NO.: 409 | SQSITGRYLAWYQQK |
| SEQ ID NO.: 410 | TLSCRASQSIT |
| SEQ ID NO.: 411 | TLSCRASQSITG |
| SEQ ID NO.: 412 | TLSCRASQSITGR |
| SEQ ID NO.: 413 | TLSCRASQSITGRY |
| SEQ ID NO.: 414 | TLSCRASQSITGRYL |
| SEQ ID NO.: 415 | TLSCRASQSITGRYLA |
| SEQ ID NO.: 416 | TLSCRASQSITGRYLAW |
| SEQ ID NO.: 417 | TLSCRASQSITGRYLAWY |
| SEQ ID NO.: 418 | TLSCRASQSITGRYLAWYQ |
| SEQ ID NO.: 419 | TLSCRASQSITGRYLAWYQQ |
| SEQ ID NO.: 420 | TLSCRASQSITGRYLAWYQQK |

Light chain CDR2 region
(SEQ ID NO: 421 to SEQ ID NO: 513)

| | |
|---|---|
| SEQ ID NO.: 421 | ESSR |
| SEQ ID NO.: 422 | ESSRV |
| SEQ ID NO.: 423 | ESSRVT |
| SEQ ID NO.: 424 | ESSRVTG |
| SEQ ID NO.: 425 | ESSRVTGI |
| SEQ ID NO.: 426 | ESSRVTGIP |
| SEQ ID NO.: 427 | ESSRVTGIPD |
| SEQ ID NO.: 428 | ESSRVTGIPDR |
| SEQ ID NO.: 429 | GESS |
| SEQ ID NO.: 430 | GESSR |
| SEQ ID NO.: 431 | GESSRV |
| SEQ ID NO.: 432 | GESSRVT |
| SEQ ID NO.: 433 | GESSRVTG |
| SEQ ID NO.: 434 | GESSRVTGI |
| SEQ ID NO.: 435 | GESSRVTGIP |
| SEQ ID NO.: 436 | GESSRVTGIPD |
| SEQ ID NO.: 437 | GESSRVTGIPDR |
| SEQ ID NO.: 438 | LLMYGE |
| SEQ ID NO.: 439 | LLMYGES |
| SEQ ID NO.: 440 | LLMYGESS |
| SEQ ID NO.: 441 | LLMYGESSR |
| SEQ ID NO.: 442 | LLMYGESSRV |
| SEQ ID NO.: 443 | LLMYGESSRVT |
| SEQ ID NO.: 444 | LLMYGESSRVTG |
| SEQ ID NO.: 445 | LLMYGESSRVTGI |
| SEQ ID NO.: 446 | LLMYGESSRVTGIP |
| SEQ ID NO.: 447 | LLMYGESSRVTGIPD |
| SEQ ID NO.: 448 | LLMYGESSRVTGIPDR |
| SEQ ID NO.: 449 | LMYGE |
| SEQ ID NO.: 450 | LMYGES |
| SEQ ID NO.: 451 | LMYGESS |
| SEQ ID NO.: 452 | LMYGESSR |
| SEQ ID NO.: 453 | LMYGESSRV |
| SEQ ID NO.: 454 | LMYGESSRVT |
| SEQ ID NO.: 455 | LMYGESSRVTG |
| SEQ ID NO.: 456 | LMYGESSRVTGI |
| SEQ ID NO.: 457 | LMYGESSRVTGIP |
| SEQ ID NO.: 458 | LMYGESSRVTGIPD |
| SEQ ID NO.: 459 | LMYGESSRVTGIPDR |
| SEQ ID NO.: 460 | MYGE |
| SEQ ID NO.: 461 | MYGES |
| SEQ ID NO.: 462 | MYGESS |
| SEQ ID NO.: 463 | MYGESSR |
| SEQ ID NO.: 464 | MYGESSRV |
| SEQ ID NO.: 465 | MYGESSRVT |
| SEQ ID NO.: 466 | MYGESSRVTG |
| SEQ ID NO.: 467 | MYGESSRVTGI |
| SEQ ID NO.: 468 | MYGESSRVTGIP |
| SEQ ID NO.: 469 | MYGESSRVTGIPD |
| SEQ ID NO.: 470 | MYGESSRVTGIPDR |
| SEQ ID NO.: 471 | RLLMYGE |
| SEQ ID NO.: 472 | RLLMYGES |
| SEQ ID NO.: 473 | RLLMYGESS |
| SEQ ID NO.: 474 | RLLMYGESSR |
| SEQ ID NO.: 475 | RLLMYGESSRV |
| SEQ ID NO.: 476 | RLLMYGESSRVT |
| SEQ ID NO.: 477 | RLLMYGESSRVTG |
| SEQ ID NO.: 478 | RLLMYGESSRVTGI |
| SEQ ID NO.: 479 | RLLMYGESSRVTGIP |
| SEQ ID NO.: 480 | RLLMYGESSRVTGIPD |
| SEQ ID NO.: 481 | RLLMYGESSRVTGIPDR |
| SEQ ID NO.: 482 | RVTG |
| SEQ ID NO.: 483 | RVTGI |
| SEQ ID NO.: 484 | RVTGIP |
| SEQ ID NO.: 485 | RVTGIPD |
| SEQ ID NO.: 486 | RVTGIPDR |
| SEQ ID NO.: 487 | SRVT |
| SEQ ID NO.: 488 | SRVTG |
| SEQ ID NO.: 489 | SRVTGI |
| SEQ ID NO.: 490 | SRVTGIP |
| SEQ ID NO.: 491 | SRVTGIPD |
| SEQ ID NO.: 492 | SRVTGIPDR |
| SEQ ID NO.: 493 | SSRV |
| SEQ ID NO.: 494 | SSRVT |
| SEQ ID NO.: 495 | SSRVTG |
| SEQ ID NO.: 496 | SSRVTGI |
| SEQ ID NO.: 497 | SSRVTGIP |
| SEQ ID NO.: 498 | SSRVTGIPD |
| SEQ ID NO.: 499 | SSRVTGIPDR |
| SEQ ID NO.: 500 | VTGI |
| SEQ ID NO.: 501 | VTGIP |
| SEQ ID NO.: 502 | VTGIPD |
| SEQ ID NO.: 503 | VTGIPDR |
| SEQ ID NO.: 504 | YGES |
| SEQ ID NO.: 505 | YGESS |
| SEQ ID NO.: 506 | YGESSR |
| SEQ ID NO.: 507 | YGESSRV |
| SEQ ID NO.: 508 | YGESSRVT |
| SEQ ID NO.: 509 | YGESSRVTG |
| SEQ ID NO.: 510 | YGESSRVTGI |
| SEQ ID NO.: 511 | YGESSRVTGIP |
| SEQ ID NO.: 512 | YGESSRVTGIPD |
| SEQ ID NO.: 513 | YGESSRVTGIPDR |

Light chain CDR3 region
(SEQ ID NO: 514 to SEQ ID NO: 628)

| | |
|---|---|
| SEQ ID NO.: 514 | ASSP |
| SEQ ID NO.: 515 | ASSPP |
| SEQ ID NO.: 516 | ASSPPT |
| SEQ ID NO.: 517 | ASSPPTY |
| SEQ ID NO.: 518 | ASSPPTYT |
| SEQ ID NO.: 519 | ASSPPTYTF |
| SEQ ID NO.: 520 | ASSPPTYTFG |
| SEQ ID NO.: 521 | ASSPPTYTFGQ |
| SEQ ID NO.: 522 | ASSPPTYTFGQG |
| SEQ ID NO.: 523 | ASSPPTYTFGQGT |
| SEQ ID NO.: 524 | AVYYCQHFASS |
| SEQ ID NO.: 525 | AVYYCQHFASSP |
| SEQ ID NO.: 526 | AVYYCQHFASSPP |
| SEQ ID NO.: 527 | AVYYCQHFASSPPT |
| SEQ ID NO.: 528 | AVYYCQHFASSPPTY |
| SEQ ID NO.: 529 | AVYYCQHFASSPPTYT |
| SEQ ID NO.: 530 | AVYYCQHFASSPPTYTF |
| SEQ ID NO.: 531 | AVYYCQHFASSPPTYTFG |
| SEQ ID NO.: 532 | AVYYCQHFASSPPTYTFGQ |
| SEQ ID NO.: 533 | AVYYCQHFASSPPTYTFGQG |
| SEQ ID NO.: 534 | AVYYCQHFASSPPTYTFGQGT |
| SEQ ID NO.: 535 | CQHFASS |
| SEQ ID NO.: 536 | CQHFASSP |
| SEQ ID NO.: 537 | CQHFASSPP |
| SEQ ID NO.: 538 | CQHFASSPPT |
| SEQ ID NO.: 539 | CQHFASSPPTY |
| SEQ ID NO.: 540 | CQHFASSPPTYT |
| SEQ ID NO.: 541 | CQHFASSPPTYTF |
| SEQ ID NO.: 542 | CQHFASSPPTYTFG |
| SEQ ID NO.: 543 | CQHFASSPPTYTFGQ |
| SEQ ID NO.: 544 | CQHFASSPPTYTFGQG |
| SEQ ID NO.: 545 | CQHFASSPPTYTFGQGT |
| SEQ ID NO.: 546 | FASS |
| SEQ ID NO.: 547 | FASSP |
| SEQ ID NO.: 548 | FASSPP |
| SEQ ID NO.: 549 | FASSPPT |
| SEQ ID NO.: 550 | FASSPPTY |
| SEQ ID NO.: 551 | FASSPPTYT |
| SEQ ID NO.: 552 | FASSPPTYTF |
| SEQ ID NO.: 553 | FASSPPTYTFG |
| SEQ ID NO.: 554 | FASSPPTYTFGQ |
| SEQ ID NO.: 555 | FASSPPTYTFGQG |
| SEQ ID NO.: 556 | FASSPPTYTFGQGT |
| SEQ ID NO.: 557 | HFASS |
| SEQ ID NO.: 558 | HFASSP |
| SEQ ID NO.: 559 | HFASSPP |
| SEQ ID NO.: 560 | HFASSPPT |
| SEQ ID NO.: 561 | HFASSPPTY |
| SEQ ID NO.: 562 | HFASSPPTYT |
| SEQ ID NO.: 563 | HFASSPPTYTF |
| SEQ ID NO.: 564 | HFASSPPTYTFG |
| SEQ ID NO.: 565 | HFASSPPTYTFGQ |
| SEQ ID NO.: 566 | HFASSPPTYTFGQG |
| SEQ ID NO.: 567 | HFASSPPTYTFGQGT |
| SEQ ID NO.: 568 | QHFASS |
| SEQ ID NO.: 569 | QHFASSP |
| SEQ ID NO.: 570 | QHFASSPP |
| SEQ ID NO.: 571 | QHFASSPPT |
| SEQ ID NO.: 572 | QHFASSPPTY |
| SEQ ID NO.: 573 | QHFASSPPTYT |
| SEQ ID NO.: 574 | QHFASSPPTYTF |
| SEQ ID NO.: 575 | QHFASSPPTYTFG |
| SEQ ID NO.: 576 | QHFASSPPTYTFGQ |
| SEQ ID NO.: 577 | QHFASSPPTYTFGQG |
| SEQ ID NO.: 578 | QHFASSPPTYTFGQGT |
| SEQ ID NO.: 579 | SPPT |
| SEQ ID NO.: 580 | SPPTY |
| SEQ ID NO.: 581 | SPPTYT |
| SEQ ID NO.: 582 | SPPTYTF |
| SEQ ID NO.: 583 | SPPTYTFG |
| SEQ ID NO.: 584 | SPPTYTFGQ |
| SEQ ID NO.: 585 | SPPTYTFGQG |
| SEQ ID NO.: 586 | SPPTYTFGQGT |
| SEQ ID NO.: 587 | SSPP |
| SEQ ID NO.: 588 | SSPPT |
| SEQ ID NO.: 589 | SSPPTY |
| SEQ ID NO.: 590 | SSPPTYT |
| SEQ ID NO.: 591 | SSPPTYTF |
| SEQ ID NO.: 592 | SSPPTYTFG |
| SEQ ID NO.: 593 | SSPPTYTFGQ |
| SEQ ID NO.: 594 | SSPPTYTFGQG |
| SEQ ID NO.: 595 | SSPPTYTFGQGT |
| SEQ ID NO.: 596 | VYYCQHFASS |
| SEQ ID NO.: 597 | VYYCQHFASSP |
| SEQ ID NO.: 598 | VYYCQHFASSPP |
| SEQ ID NO.: 599 | VYYCQHFASSPPT |
| SEQ ID NO.: 600 | VYYCQHFASSPPTY |
| SEQ ID NO.: 601 | VYYCQHFASSPPTYT |
| SEQ ID NO.: 602 | VYYCQHFASSPPTYTF |
| SEQ ID NO.: 603 | VYYCQHFASSPPTYTFG |
| SEQ ID NO.: 604 | VYYCQHFASSPPTYTFGQ |
| SEQ ID NO.: 605 | VYYCQHFASSPPTYTFGQG |
| SEQ ID NO.: 606 | VYYCQHFASSPPTYTFGQGT |
| SEQ ID NO.: 607 | YCQHFASS |
| SEQ ID NO.: 608 | YCQHFASSP |
| SEQ ID NO.: 609 | YCQHFASSPP |
| SEQ ID NO.: 610 | YCQHFASSPPT |
| SEQ ID NO.: 611 | YCQHFASSPPTY |
| SEQ ID NO.: 612 | YCQHFASSPPTYT |
| SEQ ID NO.: 613 | YCQHFASSPPTYTF |
| SEQ ID NO.: 614 | YCQHFASSPPTYTFG |
| SEQ ID NO.: 615 | YCQHFASSPPTYTFGQ |
| SEQ ID NO.: 616 | YCQHFASSPPTYTFGQG |
| SEQ ID NO.: 617 | YCQHFASSPPTYTFGQGT |
| SEQ ID NO.: 618 | YYCQHFASS |
| SEQ ID NO.: 619 | YYCQHFASSP |
| SEQ ID NO.: 620 | YYCQHFASSPP |
| SEQ ID NO.: 621 | YYCQHFASSPPT |
| SEQ ID NO.: 622 | YYCQHFASSPPTY |
| SEQ ID NO.: 623 | YYCQHFASSPPTYT |
| SEQ ID NO.: 624 | YYCQHFASSPPTYTF |
| SEQ ID NO.: 625 | YYCQHFASSPPTYTFG |
| SEQ ID NO.: 626 | YYCQHFASSPPTYTFGQ |
| SEQ ID NO.: 627 | YYCQHFASSPPTYTFGQG |
| SEQ ID NO.: 628 | YYCQHFASSPPTYTFGQGT |

Heavy chain variable domain (SEQ ID NO: 629)
QVQLVQSGAEVKKPGASVKVSCQTSGYTFTSYYMHWVRQAPGQGLEWMGLITPSGD
DTYYAQRFQGRVTMTRDTSTSPTYMELSSLTSEDTAVYYCAKMSRAGGFDVWGQGT
LVTVSS
Light chain variable domain (SEQ ID NO: 630)
EVVLTQSPGTLSLSPGERATLSCRASQSITGRYLAWYQQKPGQAPRLLMYGESSRVT
GIPDRFSGGGSGTDFTLTISRLEPEDFAVYYCQHFASSPPTYTFGQGTKLEIR
Heavy chain FR1 region (SEQ ID NO: 631)
SEQ ID NO: 631QVQLVQSGAEVKKPGASVKVSCQTSGYTFT
Heavy chain FR2 region (SEQ ID NO: 632)
SEQ ID NO: 632 WVRQAPGQGLEWMG
Heavy chain FR3 region (SEQ ID NO: 633)
SEQ ID NO: 633 RVTMTRDTSTSPTYMELSSLTSEDTAVYYCAK
Heavy chain FR4 region (SEQ ID NO: 634)
SEQ ID NO: 634 WGQGTLVTVSS
Light chain FR1 region (SEQ ID NO: 635)
SEQ ID NO: 635 EVVLTQSPGTLSLSPGERATLSC
Light chain FR2 region (SEQ ID NO: 636)
SEQ ID NO: 636 WYQQKPGQAPRLLMY
Light chain FR3 region (SEQ ID NO: 637)
SEQ ID NO: 637 GIPDRFSGGGSGTDFTLTISRLEPEDFAVYYC
Light chain FR4 region (SEQ ID NO: 638)
SEQ ID NO: 638 FGQGTKLEIR
Heavy chain CDR1 region (SEQ ID NO: 639)
SEQ ID NO.: 639 DYRIH
Heavy chain CDR2 region (SEQ ID NO: 640)
SEQ ID NO.: 640 RMNPKSGDTNFAQKFQG
Heavy chain CDR3 region (SEQ ID NO: 641)
SEQ ID NO.: 641 LLIVGGFDPLDDFEV
Light chain CDR1 region (SEQ ID NO: 642)
SEQ ID NO.: 642 SGTSSDVGGYNFVS
Light chain CDR2 region (SEQ ID NO: 643)
SEQ ID NO.: 643 EVTKRPS
Light chain CDR3 region (SEQ ID NO: 644)
SEQ ID NO.: 644 SSYGGTNNLL
Heavy chain CDR1 region (SEQ ID NO: 645)
SEQ ID NO.: 645 GYAMH
Heavy chain CDR2 region (SEQ ID NO: 646)
SEQ ID NO.: 646 VISRDARNKYYADSVKG
Heavy chain CDR3 region (SEQ ID NO: 647)
SEQ ID NO.: 647 LIIPGITEPGSPDALDI
Light chain CDR1 region (SEQ ID NO: 648)
SEQ ID NO.: 648 RASQDISKWLA
Light chain CDR2 region (SEQ ID NO: 649)
SEQ ID NO.: 649 AASSLQS
Light chain CDR3 region (SEQ ID NO: 650)
SEQ ID NO.: 650 QQASSFPWSIT
Heavy chain CDR1 region (SEQ ID NO: 651)
SEQ ID NO.: 651 SHYMH
Heavy chain CDR2 region (SEQ ID NO: 652)
SEQ ID NO.: 652 IINPSGSGTAYGQKFQG
Heavy chain CDR3 region (SEQ ID NO: 653)
SEQ ID NO.: 653 GSGGLFAY
Light chain CDR1 region (SEQ ID NO: 654)
SEQ ID NO.: 654 RASQIVRSNYLA
Light chain CDR2 region (SEQ ID NO: 655)
SEQ ID NO.: 655 GASSRAT
Light chain CDR3 region (SEQ ID NO: 656)
SEQ ID NO.: 656 LQYDSSPPTYI
Heavy chain CDR1 region (SEQ ID NO: 657)
SEQ ID NO.: 657 YFYLH
Heavy chain CDR2 region (SEQ ID NO: 658)
SEQ ID NO.: 658 IINPRGDGTRYAQKFQG
Heavy chain CDR3 region (SEQ ID NO: 659)
SEQ ID NO.: 659 GADHGAFDI
Light chain CDR1 region (SEQ ID NO: 660)
SEQ ID NO.: 660 RASQSVRRNYFA
Light chain CDR2 region (SEQ ID NO: 661)
SEQ ID NO.: 661 DASTRAT
Light chain CDR3 region (SEQ ID NO: 662)
SEQ ID NO.: 662 QQYDSSPPMYI
Heavy chain variable domain (SEQ ID NO: 663)
QMQLMQSGAEVKKPGASVTVSCKASGDTFSDYRIHWVRQAPGQGLEWMGRMNPKS
GDTNFAQKFQGRVTMTRDMSINTAYMTLSGLTFDDTALYYCASLLIVGGFDPLDDFE
VWGQGTMVTISS
Light chain variable domain (SEQ ID NO: 664)
QSALTQPPSASGSPGQSVTISCSGTSSDVGGYNFVSWYQHHPGKAPKILIYEVTKRPS
GVPDRFSGSKSGNTASLTVSGLQAEDEADYYCSSYGGTNNLLFGGGTKLTVL
Heavy chain variable domain (SEQ ID NO: 665)
QVQLVESGGGVVQPGRSLRLSCAASGLTFSGYAMHWVRQAPGKGLEWVAVISRDAR
NKYYADSVKGRFTISRDNSKKTVYLEMNSLRVEDTAVYYCAILIIPGITEPGSPDALDI
WGQGTMVSVSS
Light chain variable domain (SEQ ID NO: 666)
DIQMTQSPSSMSASVGDRVTITCRASQDISKWLAWYQQRPGKAPKLLIYAASSLQSGV
PSRFSGSGSGTDFTLTISSLQPEDFATYYCQQASSFPWSITFGQGTRLEIR

### EXAMPLES

The monoclonal antibody CV3-25 has shown in vitro protection against the original Wuhan strain of SARS-CoV-2 binding specifically to the stem helix of SARS-CoV-2, a binding region shared with antibodies of the present invention. When administered mucosally at lower dosages, CV3-25 affords a lower level of protection when compared to its systemic administration. In contrast, however, when antibodies of the present invention are administered mucosally they afford a greater level of protection as demonstrated by the in vivo studies presented below.

### Example 1: CV3-25

The aim of this study was to assess the pre-exposure efficacy of the monoclonal antibody CV3-25 following systemic intraperitoneal administration in a SARS-CoV-2 Delta K18 hACE2 Tg mouse model.

Animals (n=10 per group) were treated with an intraperitoneal dose range of the test CV3-25 (15 to 0.2 mg/kg) or PBS control as vehicle at study day −1. At study day 0, animals were challenged with 10^{3.5} TCID₅₀ of SARS-CoV-2 Delta variant.

### Materials and Methods

The monoclonal antibody CV3-25 was dissolved and diluted with phosphate-buffered saline (PBS), thereby preparing a CV3-25 dilution.

The CV3-25 dilution was made up at various concentrations so that 200 μL administrations to the mean weight per cage of the dosing group resulted in CV3-25 doses ranging from 15 mg/kg, 5 mg/kg, 1.7 mg/kg, 0.5 mg/kg to 0.2 mg/kg.

The antibody was stored at −80° C.±10° C. and dilutions were stored at 4° C. until usage and the temperature of the storage unit was monitored.

The virus strain tested was SARS-CoV-2 Delta variant (Aus/VIC/18440/2021 (B.1.617.2), which has been passed three times through Vero-hSLAM cells.

### Animals

The animal species used was the SARS-CoV-2 Delta K18 hACE2 Tg (B6.Cg-Tg (K18-ACE2) 2Prlmn/JArc; Jax™ Stock No.: 034860) mouse model, at a weight of c. 17-26 g on day −9. 60 mice were used at the age of 11-13 weeks on the day of test article administration and were 83.3% female. Ten animals were randomly allocated to 6 treatment groups based on day −9 weights to create groups with comparable mean weight, with 20% males per group, except 15 mg/kg which was exclusively female. All mice were housed in individually ventilated cages (IVCs), with corn cob bedding, tissues or shredded paper as nesting material, a wooden chew block, accessible food pellets and acidic water for animal nourishment and red plastic tunnels. Each cage system holds 3 to 5 mice per cage. All mice were transferred to fresh cages every 14 days. Food and water were inspected daily and topped up as needed.

### Study Design

The dose level of CV3-25 applied in the current example was based on a dose range proven to have prophylactic activity. A total number of 60 mice, 11-13 weeks of age were transported to the animal facility and allocated to 6 experimental groups according to Table 1 (see below). Mice were given a period of >3 days for acclimatization.

TABLE 1
Intraperitoneal experimental detail

| Test | | Admin. | Dose | Day of | No. of |
|---|---|---|---|---|---|
| group | Antibody | route | (mg/kg) | admin. | Animals |
| 15 | CV3-25 | I.P. | 15 | −1 | 10 |
| 5 | CV3-25 | I.P. | 5 | −1 | 10 |
| 1.7 | CV3-25 | I.P. | 1.7 | −1 | 10 |
| 0.5 | CV3-25 | I.P. | 0.5 | −1 | 10 |
| 0.2 | CV3-25 | I.P. | 0.2 | −1 | 10 |
| Vehicle | — | I.P. | 0 | −1 | 10 |

SARS-CoV-2 Delta K18 hACE2 mice (20% males per group, except 15 mg/kg which was exclusively female) were treated via the Intraperitoneal route of administration with the antibody at a dose between 15 mg/kg and 0.2 mg/kg based upon the average group weight at day −9 adjusted with a 4% average weight gain (based on a subset of the animals weighted at day −1). On Day 0, all mice were challenged with a lethal dose (10^{3.5} TCID₅₀) of the SARS-CoV-2 Delta variant and observed for weight loss and mortality until the end of the study at day 11.

### Antibody Administration

The test antibody was stored at −80° C.±10° C. upon arrival. The appropriate dose, according to the treatment schedule (Table 1), was formulated according to average group weight at day −9 adjusted with a 4% average weight gain (based on a subset of the animals weighted at day −1)

Prior to dosing, the material was drawn into a 1 mL syringe with a 26G needle, allowed briefly to warm to room temperature and then administered to each mouse. Mice receive the indicated dose by intraperitoneal administration of 200 μL of antibody solution into the intraperitoneal cavity, mice were scruffed and held inverted at a −45 deg angle for administration (200 μL per mouse).

### Virus Administration

The virus material was stored at −80° C.±10° C. and was defrosted prior to administration. Once defrosted, the material was diluted in sterile PBS corresponding to approximately 10^{3.5} TCID50/50 μL. As required, the animals were anesthetized by isoflurane (4% v/v with 2 L/min O₂) and each animal received approximately 50 μL (25 μl in each nare) of virus corresponding with approximately 10^{3.5} TCID₅₀ by intranasal inoculation. Unused material was frozen at −20° C. to −80° C. for back titration.

### Laboratory Analysis

Inoculum was back titrated and the dose of the virus administered was verified by titrating replicate samples on Vero cells.

### Clinical Monitoring

General health observations were performed on each animal from the day of arrival until the end of the study at least once daily (during normal servicing procedures). Each animal was weighed daily, beginning at the day of infection (day 0).

### Terminal Investigations

At the end of the study, on day 11, mice were euthanized by cervical dislocation. Gross necropsy was not performed.

### Data Analysis and Statistical Methods

Survival proportions at day 11, survival times and change in bodyweight (Area Under the Curve) were compared to the control group using Fisher's exact test, log-rank and Welch's t-test, respectively. All groups were compared to the vehicle (PBS solution) control group. P-values were adjusted according to Bonferroni (for two comparisons to vehicle) followed by a stepwise approach (starting with the highest antibody dose conditionally testing a lower dose if the previous step was statistically significant).

Statistical analysis was performed using R and statistical significance was set at α=0.05.

### Survival

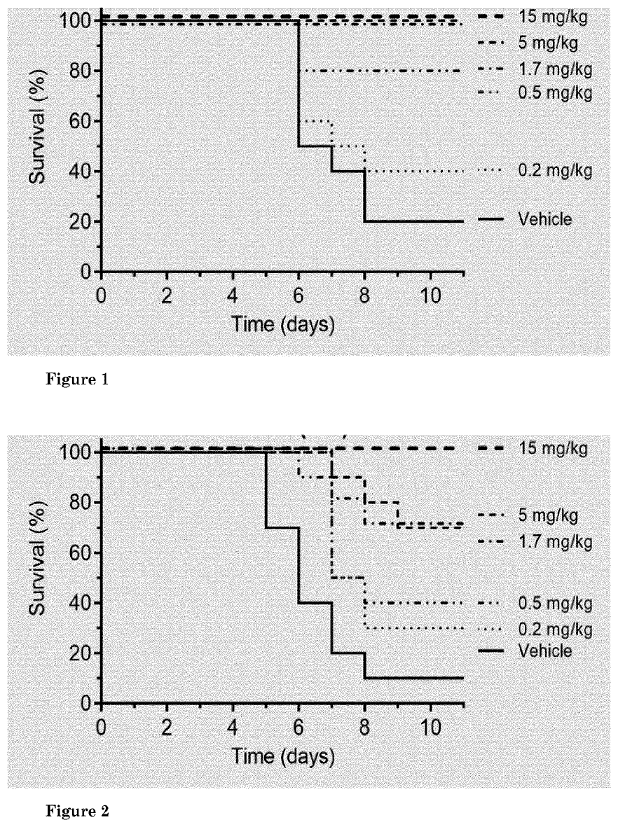

Prophylactic treatment with >0.5 mg/kg test antibody provided a statistically significant increase in survival compared to the control group (See FIG. 1). The animals that were treated with 0.5 mg/kg test antibody had a 80% survival rate, animals treated with ≥1.7 mg/kg had a survival rate of 100%, whereas the survival proportion at day 11 in the control group was 20%. The median survival time of the control group was 6.5 days.

Prophylactic treatment with ≥0.5 mg/kg test antibody resulted in a significant improvement in survival time compared to the control group.

### Body Weight

Change in bodyweight was analyzed using an Area Under the Curve (AUC) analysis in which the last observed body weight was carried forward if a mouse died/was euthanized during the study. The weight per mouse at day 0 was used as baseline and weight change was determined relative to baseline and the net AUC was defined as the summation of the area above and below the baseline using the percentage change per day. Prophylactic treatment with >0.5 mg/kg test antibody resulted in a significant reduction in weight loss compared to the control group.

### Conclusion

In this lethal SARS-CoV-2 Delta K18 hACE2 Tg mouse model, the prophylactic intraperitoneal administration of ≥0.5 mg/kg test antibody provides a statistically significant improvement in survival and a reduction in weight loss compared to control, whereas eight out of ten animals in the control group did not survive.

### Example 2: CV3-25

The aim of this study was to assess the pre-exposure efficacy of the monoclonal antibody CV3-25 following intranasal administration in a SARS-CoV-2 Delta K18 hACE2 Tg mouse model.

Animals (n=10 per group) were treated with an intranasal dose range of the test CV3-25 (15 to 0.2 mg/kg) or PBS control as vehicle on study day −1. At study day 0, animals were challenged with 10^{3.5} TCID₅₀ of SARS-CoV-2 Delta variant.

### Materials and Methods

The monoclonal antibody CV3-25 was dissolved and diluted with phosphate-buffered saline (PBS), thereby preparing a CV3-25 dilution.

The CV3-25-dilution was made up at various concentrations so that 50 μL administrations to the mean weight of the dosing group resulted in CV3-25 doses ranging from 15 mg/kg, 5 mg/kg, 1.7 mg/kg, 0.5 mg/kg to 0.2 mg/kg.

The formulations were stored at −80° C.±10° C. and the temperature of the storage unit was monitored.

The virus strain tested was SARS-CoV-2 Delta variant (Aus/VIC/18440/2021 (B.1.617.2), which had been passed three times through Vero-hSLAM cells.

### Animals

The animal species used was the SARS-CoV-2 Delta K18 hACE2 Tg (B6.Cg-Tg (K18-ACE2) 2Prlmn/Jarc; Jax™ Stock No.: 034860) mouse model, at a weight of c. 17-26 g on day −9. 60 mice were used at the age of 11-13 weeks on the day of test article administration and were 83.3% female. Ten animals were randomly allocated to 6 treatment groups, based on day −9 weights to create groups with comparable mean weight, with 20% males per group, except 15 mg/kg which was exclusively female. All mice were housed in individually ventilated cages (IVCs), with corn cob bedding, tissues or shredded paper as nesting material, a wooden chew block, accessible food pellets and acidic water for animal nourishment and red plastic tunnels. Each cage system holds 3 to 5 mice per cage. All mice were transferred to fresh cages every 14 days. Food and water were inspected daily and topped up as needed.

### Study Design

The dose level of CV3-25 applied in the current example was extrapolated from a dose range proven to have prophylactic activity intravenously. A total number of 60 mice, 11-13 weeks of age at the time of arrival, were transported to the animal facility and allocated to 6 experimental groups according to Table 2 (see below). Mice were given a period of >3 days for acclimatization.

TABLE 2
Intranasal experimental detail

| Test | | Admin. | Dose | Day of | No. of |
|---|---|---|---|---|---|
| group | Antibody | Route | (mg/kg) | admin. | Animals |
| 15 | CV3-25 | I.N. | 15 | −1 | 10 |
| 5 | CV3-25 | I.N. | 5 | −1 | 10 |
| 1.7 | CV3-25 | I.N. | 1.7 | −1 | 10 |
| 0.5 | CV3-25 | I.N. | 0.5 | −1 | 10 |
| 0.2 | CV3-25 | I.N. | 0.2 | −1 | 10 |
| Vehicle | — | I.N. | 0 | −1 | 10 |

Female SARS-CoV-2 Delta K18 hACE2 mice (20% males per group, except 15 mg/kg which was exclusively female) were treated via the intranasal route of administration with the antibody at a dose between 15 mg/kg and 0.2 mg/kg based upon the average weight at day −9 adjusted with a 4% average weight gain (based on a subset of the animals weighted at day −1). On Day 0, all mice were challenged with a lethal dose (10^{3.5} TCID₅₀) of the SARS-CoV-2 Delta variant and observed for weight loss and mortality until the end of the study at day 11.

### Antibody Administration

The test antibody was stored at −80° C.±10° C. upon arrival. The appropriate dose, according to the treatment schedule (Table 2), was formulated according to average group weight at day −9 adjusted with a 4% average weight gain (based on a subset of animals weighted at day −1).

Just prior to dosing, the material was drawn into a syringe, allowed briefly to warm to room temperature and then administered to each mouse. Mice were anaesthetized with isoflurane (4% v/v with 2 L/min O₂) for two minutes. The mice were then scruffed and held at a +45 deg angle. 50 μL of compound or vehicle control was then slowly administered to the nares of each mouse (25 μL each nare). The mice were held for an additional 5 to 10 seconds to ensure compound delivery to the lower respiratory tract before being put back into the home box.

### Virus Administration

The virus material was stored at −80° C.±10° C. and was defrosted prior to administration. Once defrosted, the material was diluted in cold PBS corresponding to approximately 10^{3.5} TCID₅₀/50 μL. As required, the animals were anesthetized with isoflurane (4% v/v with 2 L/min O₂) and each animal received approximately 50 μL (25 μL each nare) of virus corresponding with approximately 10^{3.5} TCID₅₀ by intranasal inoculation using a pipette tip. Unused material was frozen at −20° C. to −80° C. for back titration.

### Laboratory Analysis

Inoculum was returned to the lab and the dose of the virus administered was verified by titrating replicate samples on Vero cells.

### Clinical Monitoring

General health observations were performed on each animal from the day of arrival until the end of the study at least once daily (during normal servicing procedures). Each animal was weighed daily beginning one day prior to infection (day −1).

### Terminal Investigations

At the end of the study, on day 11, mice were euthanized by cervical dislocation. Gross necropsy was not performed.

### Data Analysis and Statistical Methods

Two animals in the 15 mg/kg CV3-25 were misdosed (sneezed out part of the antibody volume). These two animals were excluded from the statistical analysis.

Survival proportions at day 11, survival times and change in bodyweight (Area Under the Curve) were compared to the control group using Fisher's exact test, log-rank and Welch's t-test, respectively. All groups were compared to the vehicle (PBS solution) control group. P-values were adjusted according to Bonferroni (for two comparisons to vehicle) followed by a stepwise approach (starting with the highest antibody dose conditionally testing a lower dose if the previous step was statistically significant).

Statistical analysis was performed using R and statistical significance was set at α=0.05.

### Survival

Prophylactic treatment with ≥1.7 mg/kg test antibody provided statistically significant increase in survival compared to control group (See FIG. 2). The median survival time of the control group was 6.0 days.

Prophylactic treatment with ≥1.7 mg/kg test antibody resulted in a significant improvement in survival time compared to the control group.

### Body Weight

Change in bodyweight was analyzed using an Area Under the Curve (AUC) analysis in which the last observed body weight was carried forward if a mouse died/was euthanized during the study. The weight per mouse at day 0 was used as baseline and weight change was determined relative to baseline and the net AUC was defined as the summation of the area above and below the baseline using the percentage change per day. Prophylactic treatment with ≥0.2 mg/kg test antibody resulted in a significant reduction in weight loss compared to the control group.

### Conclusion

In this lethal SARS-CoV-2 Delta K18 hACE2 Tg mouse model, the prophylactic intranasal administration of ≥1.7 mg/kg test antibody provides a statistically significant improvement in survival and a reduction in weight loss. In contrast, the control group showed 10% survival with a median survival time of 6 days.

### Example 3: Antibody According to the Invention

The aim of this study was to assess the pre-exposure efficacy of a monoclonal antibody according to the invention, having a heavy chain CDR1 as SEQ ID NO: 029, heavy chain CDR2 as SEQ ID NO: 136, heavy chain CDR3 as SEQ ID NO: 256, a light chain CDR1 as SEQ ID NO: 372, a light chain CDR2 as SEQ ID NO: 432, and a light chain CDR3 as SEQ ID NO: 573, following systemic intraperitoneal administration in a SARS-CoV-2 Delta K18 hACE2 Tg mouse model.

Animals (n=10 per group, except the 0.5 mg/kg group where n=9) were treated with an intraperitoneal dose range of the test antibody described above (10 to 0.2 mg/kg) or PBS control as vehicle at study day −1. At study day 0, animals were challenged with 10^{3.5} TCID₅₀ of SARS-CoV-2 Delta variant.

### Materials and Methods

The monoclonal test antibody described above, was buffered in phosphate-buffered saline PBS was diluted to the final concentrations for administration (10 to 0.2 mg/kg in 200 μL). thereby preparing the test antibody dilution.

The test antibody dilution was made up at various concentrations so that 200 μL administrations to a 20 g mouse resulted in test antibody doses ranging from 10 mg/kg, 5 mg/kg, 1.7 mg/kg, 0.5 mg/kg to 0.2 mg/kg.

The antibody was stored at −80° C.±10° C. and dilutions were stored at 4° C. until usage and the temperature of the storage unit was monitored.

The virus strain tested was SARS-CoV-2 Delta variant (Aus/VIC/18440/2021 (B.1.617.2), which had been passed three times through Vero-hSLAM cells.

### Animals

The animal species used was the SARS-CoV-2 Delta K18 hACE2 Tg (B6.Cg-Tg (K18-ACE2) 2Prlmn/JArc; Jax™ Stock No.: 034860) mouse model, at a weight of c. 16.7-22.4 g at day of viral challenge. 59 mice were used at the age of 7-11 weeks of age and were exclusively female. Ten animals were allocated to 5 treatment groups, and nine animals to the 0.5 mg/kg group. All mice were housed in individually ventilated cages (IVCs), with corn cob bedding, tissues or shredded paper as nesting material, a wooden chew block, accessible food pellets and acidic water for animal nourishment and red plastic tunnels. Each cage system holds 3 to 5 mice per cage. All mice were transferred to fresh cages every 14 days. Food and water were inspected daily and topped up as needed. Each animal was weighed daily beginning at the day of infection (day 0).

### Study Design

The dose level of test antibody applied in the current example was based on the maximal dosing possible for the material stock concentration (4 mg/mL) and based on literature and previous experience with an anti-S2 antibody (CV3-25) showing protection IP at doses ≥0.5 mg/kg. A total number of 59 mice, 7-11 weeks of age, were allocated to 6 experimental groups according to Table 3 (see below). Mice were given a period of >3 days for acclimatization.

TABLE 3
Intraperitoneal experimental detail

| Test | | Admin. | Dose | Day of | No. of |
|---|---|---|---|---|---|
| group | Antibody | route | (mg/kg) | admin. | Animals |
| 10 | As disclosed | I.P. | 10 | −1 | 10 |
| 5 | above. | I.P. | 5 | −1 | 10 |
| 1.7 | | I.P. | 1.7 | −1 | 10 |
| 0.5 | | I.P. | 0.5 | −1 | 9 |
| 0.2 | | I.P. | 0.2 | −1 | 10 |
| Vehicle | — | I.N. | 0 | −1 | 10 |

Female SARS-CoV-2 Delta K18 hACE2 mice were treated via the intraperitoneal route of administration with the antibody at a dose between 10 mg/kg and 0.2 mg/kg based upon a mouse weight of 20 g. On Day 0, all mice were challenged with a lethal dose (10^{3.5} TCID₅₀) of the SARS-CoV-2 Delta variant and observed for weight loss and mortality until the end of the study at day 12.

### Antibody Administration

The test antibody was stored at −80° C.±10° C. upon arrival. The appropriate dose, according to the treatment schedule (Table 3), was formulated assuming a weight of 20 g per mouse.

Prior to dosing, the material was drawn into a 1 ml syringe with a 26G needle and then administered to each mouse. Mice in the treatment group received the indicated dose by intraperitoneal administration of 200 μL of antibody solution into the intraperitoneal cavity, mice were scruffed and held inverted at a −45 deg angle for administration (200 μL per mouse). Mice in the vehicle group that received PBS were anaesthetized with isoflurane (4% v/v with 2 L/min O₂) for two minutes. The mice were then scruffed and held at a +45 deg angle. 50 μL of compound or vehicle control was then slowly administered to the nares of each mouse (25 μL each nare). The mice were held for an additional 5 to 10 seconds to ensure compound delivery to the lower respiratory tract before being put back into the home box.

### Virus Administration

The virus material was stored at −80° C.±10° C. and was defrosted prior to administration, at a titer of 10^{6.3} TCID₅₀/mL. Once defrosted, the material was diluted in sterile PBS corresponding to approximately 10^{3.5} TCID₅₀/50 μL). As required, the animals were anesthetized by isoflurane (4% v/v with 2 L/min O₂) and each animal received approximately 50 μL (25 μL in each nare) of virus corresponding with approximately 10^{3.5} TCID₅₀ by intranasal inoculation. Unused material was frozen at −20° C. to −80° C. for back titration.

### Laboratory Analysis

Inoculum was back titrated and the actual dose of the virus administered was verified by titrating replicate samples on Vero cells.

### Terminal Investigations

At the end of the study, on day 12, mice were euthanized by cervical dislocation. Gross necropsy was not performed.

### Data Analysis and Statistical Methods

Survival proportions at day 12, survival times and change in bodyweight (Area Under the Curve) were compared to the vehicle (PBS solution) control group using Fisher's exact test, log-rank and Welch's t-test, respectively. P-values were adjusted according to Bonferroni (for two comparisons to vehicle) followed by a stepwise approach (starting with the highest antibody dose conditionally testing a lower dose if the previous step was statistically significant).

Statistical analysis was performed using R and statistical significance was set at α=0.05.

### Survival—Intraperitoneal

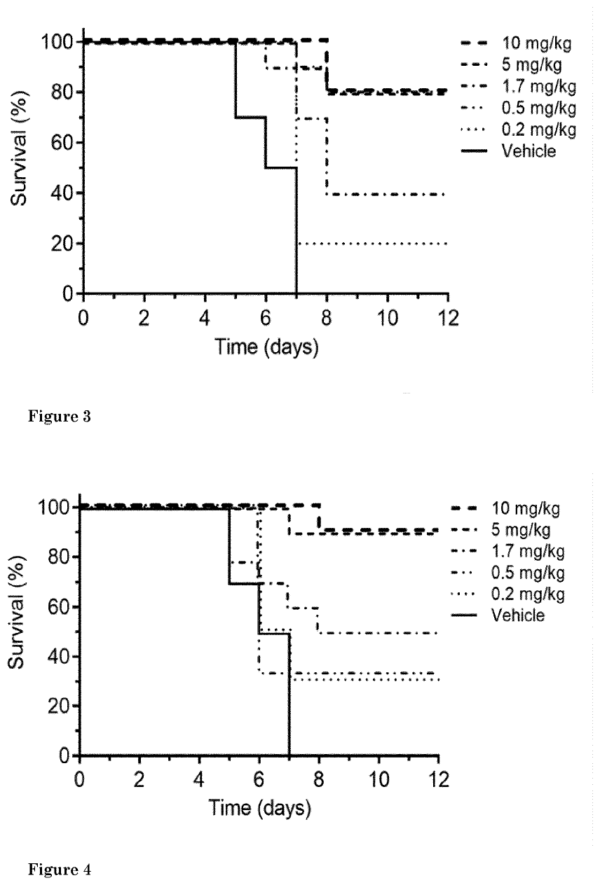

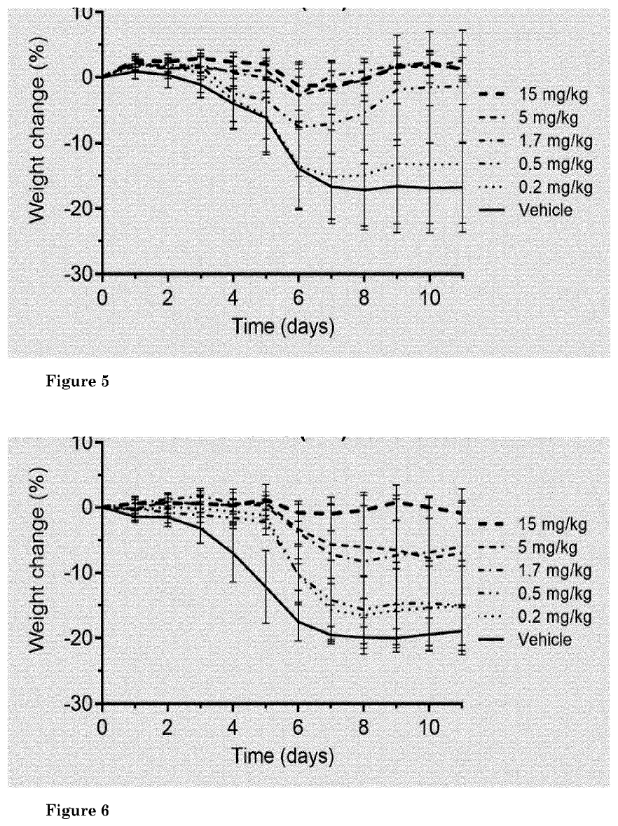

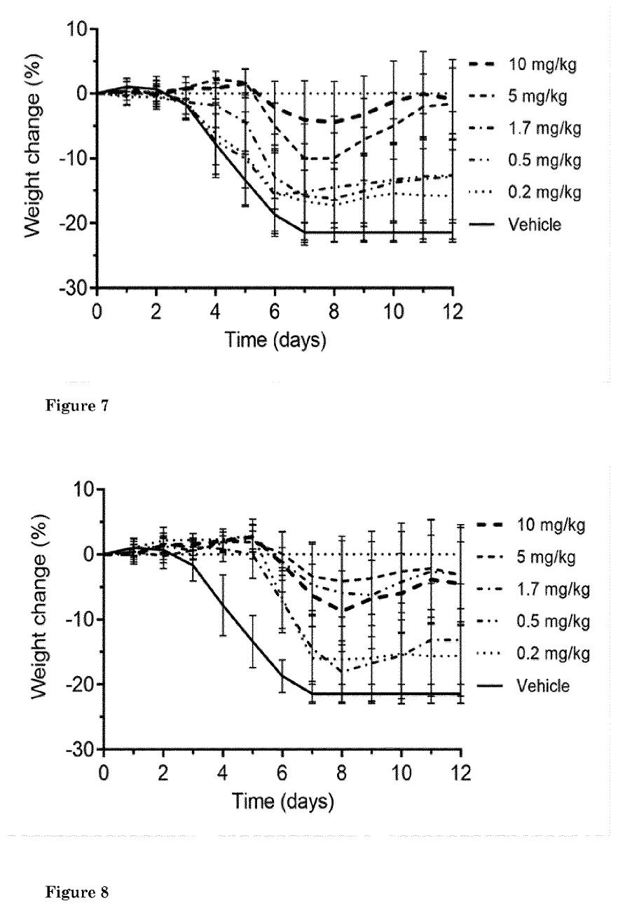

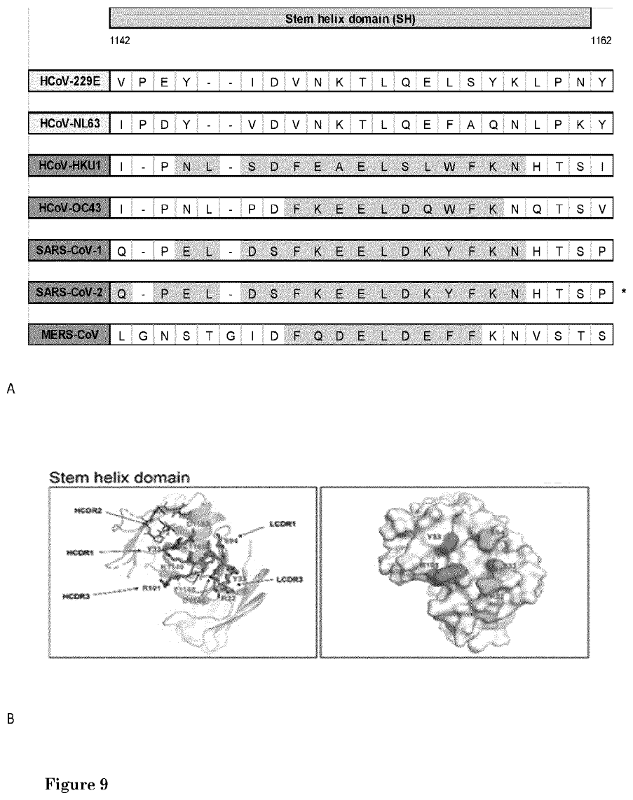

Prophylactic treatment with ≥5 mg/kg test antibody provides statistically significant increase in survival compared to vehicle (See FIG. 3). The animals that were treated with 5 and 10 mg/kg of the test antibody had a 90% survival rate, whereas the survival proportion at day 7 in the control group was 0%. The median survival time of the control group was 6.5 days.

Prophylactic treatment with ≥1.7 mg/kg test antibody resulted in a significant improvement in survival time.

### Body Weight

Change in bodyweight was analyzed using an Area Under the Curve (AUC) analysis in which the last observed body weight was carried forward if a mouse died/was euthanized during the study. The weight per mouse at day 0 was used as baseline and weight change was determined relative to baseline and the net AUC was defined as the summation of the area above and below the baseline using the percentage change per day. Prophylactic treatment with ≥1.7 mg/kg test antibody resulted in a significant reduction in weight loss compared to the control group.

### Conclusion

In this lethal SARS-CoV-2 Delta K18 hACE2 Tg mouse model, the prophylactic intraperitoneal administration of ≥5 mg/kg test antibody provides significant improvement in survival compared to vehicle and ≥1.7 mg/kg a reduction in weight loss whereas all the animals in the control group died.

### Example 4: Antibody According to the Invention

The aim of this study was to assess the pre-exposure efficacy of a monoclonal antibody according to the invention, having a heavy chain CDR1 as SEQ ID NO: 029, heavy chain CDR2 as SEQ ID NO: 136, heavy chain CDR3 as SEQ ID NO: 256, a light chain CDR1 as SEQ ID NO: 372, a light chain CDR2 as SEQ ID NO: 432, and a light chain CDR3 as SEQ ID NO: 573, following intranasal administration in a SARS-CoV-2 Delta K18 hACE2 Tg mouse model.

Animals (n=10 per group) were treated with an intranasal dose range of the test antibody described above (10 to 0.2 mg/kg) or PBS control at study day −1. At study day 0, animals were challenged with 10^{3.5} TCID₅₀ of SARS-CoV-2 Delta variant.

### Materials and Methods

The monoclonal antibody according to the invention was buffered in phosphate-buffered saline PBS was diluted to the final concentrations for administration (10 to 0.2 mg/kg in 50 μL)

The monoclonal dilution was made up at various concentrations so that 50 μL administrations to a 20 g mouse resulted in test antibody doses ranging from 10 mg/kg, 5 mg/kg, 1.7 mg/kg, 0.5 mg/kg to 0.2 mg/kg.

The antibody was stored at −80° C.±10° C. and dilutions were stored at 4° C. until usage and the temperature of the storage unit was monitored.

The virus strain tested was SARS-CoV-2 Delta variant (Aus/VIC/18440/2021 (B.1.617.2), which had been passed three times through Vero-hSLAM cells.

### Animals

The animal species used was the SARS-CoV-2 Delta K18 hACE2 Tg mouse model, at a weight of c. 17.2-21.9 g on the day of test article administration. 60 mice were used at the age of 7-11 weeks and were exclusively female. Ten animals were allocated to 6 treatment groups based on creating groups with similar mean weight and weight variability based on Day-5 weights. All mice were housed in individually ventilated cages (IVCs), with corn cob bedding, tissues or shredded paper as nesting material, a wooden chew block, accessible food pellets and acidic water for animal nourishment and red plastic tunnels. Each cage system holds 3 to 5 mice per cage. All mice were transferred to fresh cages every 14 days. Food and water were inspected daily and topped up as needed. Each animal was weighed daily beginning at the day of infection (Day 0).

### Study Design

The dose level of the monoclonal antibody applied in the current example was based on the maximal dosing possible for the material stock concentration (4 mg/mL) and based on literature and previous experience with an anti-S2 antibody (CV3-25) showing protection IN at doses ≥1.7 mg/kg. A total number of 60 mice, 7-11 weeks of age were transported to the animal facility and allocated to 6 experimental groups according to Table 4 (see below). Mice were given a period of >3 days for acclimatization.

TABLE 4
Intranasal experimental detail

| Test | | Admin. | Dose | Day of | No. of |
|---|---|---|---|---|---|
| group | Antibody | Route | (mg/kg) | admin. | Animals |
| 10 | As disclosed | I.N. | 10 | −1 | 10 |
| 5 | above. | I.N. | 5 | −1 | 10 |
| 1.7 | | I.N. | 1.7 | −1 | 10 |
| 0.5 | | I.N. | 0.5 | −1 | 10 |
| 0.2 | | I.N. | 0.2 | −1 | 10 |
| Vehicle | — | I.N. | 0 | −1 | 10 |

Female SARS-CoV-2 Delta K18 hACE2 mice were treated via the intranasal route of administration with the antibody at a dose between 10 mg/kg and 0.2 mg/kg based upon a mouse weight of 20 g. On Day 0, all mice were challenged with a lethal dose (10^{3.5} TCID₅₀) of the SARS-CoV-2 Delta variant and observed for survival and weight loss until the end of the study at day 12.

### Antibody Administration

The test antibody was stored at −80° C.±10° C. upon arrival. The appropriate dose, according to the treatment schedule (Table 4), was formulated assuming a mouse weight of 20 g.

Just prior to dosing, the material was drawn into a 1 ml syringe with a 26G needle, and then administered to each mouse. Mice were anaesthetized with isoflurane (4% v/v with 2 L/min O₂) for two minutes. The mice were then scruffed and held at a +45 deg angle. 50 μL of compound or vehicle control was then slowly administered to the nares of each mouse (25 μL each nare). The mice were held for an additional 5 to 10 seconds to ensure compound delivery to the lower respiratory tract before being put back into the home box.

### Virus Administration

The virus material was stored at −80° C.±10° C. and was defrosted prior to administration. Once defrosted, the material was diluted in cold PBS corresponding to approximately 10^{3.5} TCID₅₀/50 μL. As required, the animals were anesthetized by isoflurane (4% v/v with 2 L/min O₂) and each animal received approximately 50 μL (25 μL each nare) of virus corresponding with approximately 10^{3.5} TCID₅₀ by intranasal inoculation. Unused material was frozen at −20° C. to −80° C. for back titration.

### Laboratory Analysis

Inoculum was back titrated and the dose of the virus administered was verified by titrating replicate samples on Vero cells.

### Terminal Investigations

At the end of the study, on day 12, mice were euthanized by cervical dislocation. Gross necropsy was not performed.

### Data Analysis and Statistical Methods

Survival proportions at day 12, survival times and change in bodyweight (Area Under the Curve) were compared to the vehicle (PBS solution) control group using Fisher's exact test, log-rank and Welch's t-test, respectively. P-values were adjusted according to Bonferroni (for two comparisons to vehicle) followed by a stepwise approach (starting with the highest antibody dose conditionally testing a lower dose if the previous step was statistically significant).

Statistical analysis was performed using R and statistical significance was set at α=0.05.

### Survival—Intranasal

Prophylactic treatment with ≥5 mg/kg test antibody provides statistically significant increase in survival compared to vehicle (See FIG. 4). The animals that were treated with 5 and 10 mg/kg test antibody had a 80% survival rate, whereas the survival proportion at day 7 in the control group was 0%. The median survival time of the control group was 6.5 days.

Prophylactic treatment with ≥0.2 mg/kg test antibody resulted in a significant improvement in survival time.

### Body Weight

Change in bodyweight was analyzed using an Area Under the Curve (AUC) analysis in which the last observed body weight was carried forward if a mouse died/was euthanized during the study. The weight per mouse at day 0 was used as baseline and weight change was determined relative to baseline and the net AUC was defined as the summation of the area above and below the baseline using the percentage change per day. Prophylactic treatment with ≥0.2 mg/kg test antibody resulted in a significant reduction in weight loss compared to the control group.

### Conclusion

In this lethal SARS-CoV-2 Delta K18 hACE2 Tg mouse model, the prophylactic intranasal administration of ≥5 mg/kg test antibody provides significant improvement in survival and ≥0.2 mg/kg a reduction in weight loss compared to vehicle. In contrast, all the animals in the control group had died by day 7.

### Example 5: Bi-Specific Antibody According to the Invention

The aim of this intranasal murine study is to assess the pre- and post-exposure efficacy of a bispecific monoclonal antibody in comparison with the parental antibodies, according to the invention, having a first Fab capable of binding to the stem helix of SARS-CoV-2 and a second Fab capable of binding to the fusion peptide of SARS-CoV-2. The first parental antibody (“Parental mAb 1”) has the following CDRs as described in SEQ ID Numbers: 029, 136, 256, 372, 432 and 573. The second parental antibody (“Parental mAb 2”) has the following CDRs as described in SEQ ID Numbers: 645, 646, 647, 648, 649 to 650. The bi-specific antibody according to the invention has a first Fab that comprises a sequence comprising any one or more of SEQ ID Numbers: 029, 136, 256, 372, 432 or 573; wherein the second Fab comprises a sequence comprising any one or more of SEQ ID Numbers: 645, 646, 647, 648, 649 to 650.

Animals are treated with an intranasal nominal dose (0.001 mg/kg to 15 mg/kg) comprising either Parental mAb 1, or Parental mAb 2, or the bispecific antibody described above or vehicle control at study day −1.

### Materials and Methods

The monoclonal parental and bispecific antibodies according to the invention is provided in a sodium acetate buffer (e.g. 20 mM Sodium Acetate, 75 mM Sodium Chloride, 5% sucrose, pH 5.5) and is diluted to the final concentration for administration ranging from 0.001 mg/kg to 15 mg/kg, administered as 25 μL to 50 μL per nare.

The mice allocated to the control group (Sodium acetate buffer) are administered with vehicle (e.g. 20 mM Sodium Acetate, 75 mM Sodium Chloride, 5% sucrose, pH 5.5), administered as 25 μL to 50 μL per nare.

All mice receive intranasal challenge with SARS-CoV-2 Delta at day 0.

### Animals

The SARS-CoV-2 Delta K18 hACE2 Tg mice are used at a weight of c. 15 g to 22 g (e.g. 20 g per animal) on commencement of the study. Between 6 and 10 animals are allocated to each treatment group.

### Study Design

The dose level of the monoclonal and bispecific antibodies applied in the current example is based on the maximal dosing and based upon previous results from efficacy studies in mice with the parental antibodies alone. From the maximal dose of ≤15 mg/kg, a 3- or 4-fold dilution curve for each the parental or bispecific antibody is applied.

TABLE 5
Intranasal experimental detail per antibody

| Test | | Dose | Day of | No. of |
|---|---|---|---|---|
| group | Antibody | (mg/kg) | admin. | Animals |
| 1 | Parental mAb 1 | 0.001-15 | −1 | 6-10 |
| | (dose 1) | (e.g. 0.0027, 0.063) | | |
| 2 | Parental mAb 1 | 0.001-15 | −1 | 6-10 |
| | (dose 2) | (e.g. 0.106, 0.189) | | |
| 3 | Parental mAb 1 | 0.001-15 | −1 | 6-10 |
| | (dose 3) | (e.g. 0.425, 0.567) | | |
| 4 | Parental mAb 1 | 0.001-15 | −1 | 6-10 |
| | (dose 4) | (e.g. 1.7) | | |
| 5 | Parental mAb 2 | 0.001-15 | −1 | 6-10 |
| | (dose 1) | (e.g. 0.0027, 0.063) | | |
| 6 | Parental mAb 2 | 0.001-15 | −1 | 6-10 |
| | (dose 2) | (e.g. 0.106, 0.189) | | |
| 7 | Parental mAb 2 | 0.001-15 | −1 | 6-10 |
| | (dose 3) | (e.g. 0.425, 0.567) | | |
| 8 | Parental mAb 2 | 0.001-15 | −1 | 6-10 |
| | (dose 4) | (e.g. 1.7) | | |
| 9 | Bispecific mAb | 0.001-15 | −1 | 6-10 |
| | (dose 1) | (e.g. 0.007, 0.005) | | |
| 10 | Bispecific mAb | 0.001-15 | −1 | 6-10 |
| | (dose 2) | (e.g. 0.027, 0.020) | | |
| 11 | Bispecific mAb | 0.001-15 | −1 | 6-10 |
| | (dose 3) | (e.g. 0.106, 0.080) | | |
| 12 | Bispecific mAb | 0.001-15 | −1 | 6-10 |
| | (dose 4) | (e.g. 0.425, 0.310) | | |
| 13 | Bispecific mAb | 0.001-15 | −1 | 6-10 |
| | (dose 5) | (e.g. 1.7, 1.25) | | |
| 14 | Bispecific mAb | 0.001-15 | −1 | 6-10 |
| | (dose 6) | (e.g. 5.0) | | |
| Vehicle | — | 0 | −1 | 6-10 |

The mice are treated via the intranasal route of administration with compositions comprising either parental antibodies or the bispecific antibody at dose ranges between 0.001 and 15 mg/kg. On Day 0, all mice are challenged with a dose of the SARS-CoV-2 and observed for survival and body weight until the end of the study (e.g. Day 10-15).

### Antibody Administration

The parental monoclonal antibodies or bispecific antibody or vehicle only is administered to each nare (e.g. pipette or spray) according to the treatment schedule (Table 5), using volumes of between 25 μL to 50 μL per nare.

### Virus Administration

The virus material is defrosted prior to administration. Once defrosted, the material is diluted and each animal received between 25 μL to 50 μL of virus per nare. The mice are infected with between 10² and 10⁸ TCID₅₀ of SARS-CoV-2 (e.g. 10^{3.5} TCID₅₀ of SARS-CoV-2).

### Laboratory Analysis

Inoculum is returned to the lab and the dose of the virus administered is verified by titrating replicate samples on Vero cells.

### Clinical Monitoring

General health observations are performed on each animal from the day of arrival until the end of the study at least once daily (during normal servicing procedures). Each animal is weighed daily beginning one day prior to infection (day −1).

### Data Analysis and Statistical Methods

Survival proportions and survival times and change in bodyweight (Area Under the Curve) are compared to the corresponding control group using Fisher's exact test, log-rank and Welch's t-test, respectively. All groups are compared to the vehicle control group. P values are adjusted according to Bonferroni (for three comparisons to vehicle) followed by a stepwise approach within antibody (starting with the highest dose and conditionally testing a lower dose if the previous step was statistically significant).

Survival dose response curves are fitted for each of the treatments (Parental mAb 1, Parental mAb 2 and Bispecific) and the ED₅₀ is estimated. The efficacy of the bispecific antibody is then compared to the parental antibodies.

### Survival

Prophylactic treatment with Parental mAb 1, Parental mAb 2 or bispecific antibody (as indicated in Table 5) compared to the control group provides statistically significant protection against mortality and a significant improvement in survival time compared to the control group.

### Body Weight

Change in bodyweight is analyzed using an Area Under the Curve (AUC) analysis in which the last observed body weight is carried forward if a mouse died/is euthanized during the study. The weight per mouse at day 0 is used as baseline and weight change is determined relative to baseline with the net AUC defined as the summation of the area above and below the baseline using the percentage change per day.

Prophylactic treatment with the parental or bispecific antibodies (as indicated in Table 5) provides a statistically significant reduction in weight loss, compared to the control group.

### Conclusion

In this SARS-CoV-2 Delta mouse model, the prophylactic intranasal administration of a bispecific antibody (as indicated in Table 5) according to the invention provides significant improvement in survival and a reduction in body weight loss compared with Control Group. The outcome of the combination index is suggestive of at least a comparable, an additive or potentially synergistic effect for the bispecific compared to the parental antibodies.

### Example 6: ELISA Binding Assessment of an Antibody According to the Invention

The objective of this study was to assess the binding of the antibody against different Corona Spike antigens, a variety of alpha and beta coronaviruses.

Binding was tested in an enzyme-linked immunosorbent assay (ELISA). Briefly, a Spike antigen was used to coat the surface of a microwell of an ELISA plate. The antibody was then added in decreasing concentration to the coated and pre-blocked microwell surface in duplicates, attaching to the coated antigen. A detection antibody was added, labeled with horse radish peroxide (HRP), that then generates a colorimetric signal upon addition of the substrate. The readout was optical density and reflects binding. The antibody was assayed in duplicate against a number of alpha and beta coronavirus spike antigens, including but not limited to SARS-CoV-2 Delta, Omicron XBB.1.5 and human coronavirus NL63. The antibody was tested in comparison to other anti-S1 antibodies in the range of 10.0-0.000002 nM. Half maximal effective concentration (EC₅₀) was calculated for each antibody on each antigen as an estimate of binding affinity.

TABLE 6
ELISA EC₅₀ values (nM) for the antibody according to invention.

| | CoV-2 | CoV-2 | |
|---|---|---|---|
| | Delta | XBB.1.5 | NL63 |
| COV30-14 | 0.00031 | 0.000057 | N/D |
| Anti-S1 antibody | 6.9 | N/D | N/D |
| DZIF-10c | | | |
| Anti-S1 mAb | 0.010 | N/D | N/D |
| ImdevimAb | | | |
| Human IgG1 | N/D | N/D | N/D |
| Isotype | | | |

N/D = no EC₅₀value was determined, due to lack of binding.

The ELISA binding assay (Table 6) showed that the antibody according to invention was able to effectively bind all tested Spike antigens from beta coronaviruses, including SARS-CoV-2 Delta and Omicron XBB.1.5. Alpha coronavirus NL63 spike antigen was not bound by the antibody. The anti-S1 antibodies tested only bound the SARS-CoV-2 Delta and not the Omicron mutant, as expected from literature and did not show binding amongst alpha coronavirus NL63. The isotype control antibody did not show any binding to the coronavirus antigens.

### Example 7: Affinity Binding Assessment by MSD of an Antibody According to the Invention

The objective of this study was to assess relative binding affinity against Spike antigens in a multiplexed way, with very high specificity and low sample input. Therefore, an antibody was tested in a multiplexed assay, Meso Scale Discovery (MSD), which uses electrochemiluminescent labels that were conjugated to detection antibodies. In this assay, up to ten trimeric viral antigens are separately printed onto a spot each well of the plate, allowing for a multiplexed assay readout. The antibody according to the invention bind the viral antigens through Fab-mediated recognition, then an anti-human IgG Sulfo-Tag detection antibody is added, recognizing human IgG Fab. Upon addition of read buffer containing substrate, and running a current through the plate electrodes, an electro-(current) chemi-(buffer substrate) luminescent (light) cascade is initiated which results in light emission. The intensity of emitted light is measured per spot, revealing bound levels of analyte. The antibody was assayed in duplicate against Spike antigens of SARS-CoV-2, SARS-CoV-1, MERS, hCoV NL63, hCoV HKU, hCoV OC43 and hCoV 229E (V-Plex COVID-19 Coronavirus Panel 3 (IgG) Kit cat #K15399U-2) (Table 7), as well as a set of Omicron variants (V-Plex SARS-CoV-2 Panel 34 (IgG) Kit cat #K15690U-2) in a separate assay (Table 8). The antibody was tested in a dilution range starting from 555 ng/ml for the Panel 3 and 10 ng/ml in the Omicron Panel 24 kit. Light emission from the MSD sulfo-tag antibody was quantified with MSD Discovery workbench, calibration curves were used to calculate antibody concentrations, by fitting the raw electrochemiluminescent units (eCLU) from the calibrators to a logistic regression for curve fitting (Sigmoidal, 4PL, X=conc), and the model asymptotes were constrained to the lower limit of detection (LLOQ) and the upper limit of detection (ULOQ). The concentration (ng/ml) corresponding to the midpoint of the dynamic range (ie the range between the lower and upper limit of quantification, LLOQ and ULOQ) was reported.

TABLE 7
Antibody concentrations corresponding to the midpoint of the dynamic range
(LOQ + ULOQ)/2 (ng/ml) for the antibody according to invention against
different alpha and beta coronaviruses is shown.

| | | | | | | | | SARS- |
|---|---|---|---|---|---|---|---|---|
| | | | | | | | | CoV-2 |
| | SARS- | SARS- | MERS | hCoV- | hCoV- | hCoV- | hCoV- | S1 |
| | CoV-2 | CoV-1 | -CoV | OC43 | HKU1 | NL63 | 229E | RBD |
| COV30-14 | 0.52 | 0.26 | 0.16 | 3.72 | 1.85 | N/D | N/D | N/D |
| Anti-S1 mAb | 0.16 | N/D | N/D | N/D | N/D | N/D | N/D | 0.09 |
| DZIF-10c | | | | | | | | |
| isotype | N/D | N/D | N/D | N/D | N/D | N/D | N/D | N/D |
| control | | | | | | | | |

N/D = antibody concentration could not be determined.

The MSD binding array (see Table 7) showed that the antibody according to the invention was able to effectively bind all Spike antigens from beta coronaviruses, including SARS-CoV-2, MERS, HKU1 and OC43. Alpha coronaviruses including 229E and NL63 and SARS-CoV-2 receptor binding domain (RBD) on the S1 part of the Spike were not bound by the antibody. The anti-S1 antibody tested here bound the RBD domain and the wildtype SARS-CoV-2 very well, as expected from literature and did not show binding breadth amongst other tested alpha coronaviruses. This suggests that the antibody according to invention shows efficient binding across all tested beta coronavirus Spikes, while the anti-RBD antibody only bound SARS-CoV-2 Spike and RBD domain. The isotype control antibody did not show any binding to the coronavirus antigens.

TABLE 8
Antibody concentrations corresponding to the midpoint of the dynamic range (LOQ + ULOQ)/2 (pg/ml)
for antibody according to the invention against different SARS-CoV-2 Omicron viruses is shown.

| | | SARS- | SARS- | SARS- | SARS- | SARS- | SARS- | SARS- | SARS- | SARS- |
|---|---|---|---|---|---|---|---|---|---|---|
| | SARS- | CoV-2 | CoV-2 | CoV-2 | CoV-2 | CoV-2 | CoV-2 | CoV-2 | CoV-2 | CoV-2 |
| | CoV-2 | BA.1 | BA.2.75 | BA.5 | BF.7 | BN.1 | BQ.1 | BQ.1.1 | XBB.1 | XBB.1.5 |
| COV30-14 | 540 | 105 | 139 | 155 | 131 | 102 | 168 | 151 | 157 | 154 |
| Anti-S1 mAb | 117 | 93 | 81 | 630 | 13625 | 11558 | N/D | N/D | N/D | N/D |
| DZIF-10c | | | | | | | | | | |
| isotype | N/D | N/D | N/D | N/D | N/D | N/D | N/D | N/D | N/D | N/D |
| control | | | | | | | | | | |

N/D = antibody concentration could not be determined.

The MSD binding array showed that the antibody according to invention can effectively bind all Omicron variants tested in this assay, at even lower concentrations than SARS-CoV-2 Wuhan (Table 8), therefore retaining activity among the Omicron mutants. The SARS-CoV-2 BA.1 and BN.1 variants were bound at especially low concentrations. While the anti-S1 control antibody bound the SARS-CoV-2 Spike at lower concentrations than the antibody according to invention, the earlier Omicron mutants were only bound at very high concentrations and the later Omicron mutants such as BQ.1 and XBB.1 were not bound. This indicates that the antibody according to invention was able to bind to the latest Omicron mutants while the anti-RBD antibody did not bind these variants. The isotype control antibody did not show any binding to the coronavirus antigens.

### Example 8. Live Virus Neutralization of an Antibody According to the Invention

The objective of this study was to assess the ability of an antibody to neutralize live corona viruses. The antibody was tested for functional activity in a live virus microneutralization assay against MERS, SARS-CoV-1 and SARS-CoV-2 Wuhan. Briefly, dilution series of an antibody were pre-incubated with the corresponding virus and then added to the respective cell line (Vero: MERS and SARS-CoV-1, SARS-CoV-2: Vero E6 cells) in quadruplicates. After incubation, cells were fixed, stained with an anti-nucleocapsid antibody, and an enzymatically tagged detection antibody was added. Colored precipitate signaling nucleocapsid presence was read out via an Immunospot analyzer and 50% inhibitory concentration (IC₅₀), was reported via the Zielinska method (REF: https://doi.org/10.1186/1743-422X-2-84). The antibody was tested for neutralizing activity in the range of 0.025 to 500 μg/ml.

TABLE 9
Live virus neutralization assay. IC₅₀ values in μg/ml are
displayed for against three different beta coronaviruses.
A pooled convalescent serum from SARS-COV-2 patients
was used as a comparator and SARS-COV-2 positive control,
MN₅₀ titers are shown.

| | SARS- | | |
|---|---|---|---|
| | COV-2 | SARS- | MERS- |
| Live virus neutralization | Wuhan | COV-1 | COV |
| COV30-14 | 25.45 | 4.95 | 3.68 |
| SARS-COV-2 pooled | 142.00 | N/D | N/D |
| convalescent serum | | | |

N/D = MN₅₀ could not be determined

Overall, the antibody according to the invention was able to neutralize the tested viruses SARS-CoV-1, SARS-CoV-2 and MERS-COV at varying concentrations (see Table 9), with the lowest IC₅₀ concentrations for MERS-COV. This confirms that the antibody has neutralizing activity across these beta coronaviruses, as indicated by the binding assessments. The convalescent serum was only able to neutralize SARS-CoV-2 Wuhan at high MN₅₀ titers.

### Example 9. Pseudovirion Neutralization of an Antibody According to the Invention

The objective of this study was to assess the ability of an antibody to neutralize different pseudotyped virus particles. The advantage over a live virus assay was that pseudotyped viruses can be used at a lower biosafety level laboratory and new variants of concern can be produced and tested more rapidly, allowing for broader testing across variants of concern or interest. Briefly, pseudovirions were produced by co-transfecting virus expression plasmids with the pHIV-1NL43 ΔEnv-NanoLuc reporter virus plasmid in HEK293T cells. Then, dilution series of the antibody were pre-incubated with the corresponding pseudovirus and then added to HEK 293T cells expressing ACE2, the entry receptor for SARS-CoV-2, SARS-CoV-1 and NL63. For hCoV 229E, the diluted antibody mixed with virus was added to Huh7 cells which express the aminopeptidase N (APN receptor which facilitates entry of hCoV 229E into host cells. After incubation, cells were washed and lysed to measure the luciferase activity in cell lysates using the Nano-Glo Luciferase Assay System and GloMax system, with a readout of relative light units (RLUs). The 50% inhibitory concentrations (IC₅₀) were determined as the antibody concentration at which infectivity was inhibited by 50% using a four Parameter Logistic Regression (4PL) curve fit. The bispecific antibody was tested for neutralizing activity in the range of 0.0042 to 250 μg/ml against SARS-CoV-1, SARS-CoV-2 Wuhan, Delta, Omicron BA.4, hCoV NL63 and hCoV 229E.

TABLE 10
Pseudovirion neutralization assay. IC₅₀ values in μg/ml were displayed for
the anti-stem helix antibody against different alpha and beta coronaviruses.
For multiple runs, the geometric mean with the 95% normal range is shown.

| | | SARS- | SARS- | SARS- | SARS- | SARS- | | |
|---|---|---|---|---|---|---|---|---|
| | SARS- | CoV-2 | CoV-2 | CoV-2 | CoV-2 | CoV-2 | CoV | CoV |
| | CoV-1 | Wuhan | Delta | BA4 | XBB.1 | BQ.1.1 | 229E | NL63 |
| COV30-14 | 5.887 | 0.211 | 14.374 | 7.601 | 9.915 | 9.625 | >250 | >250 |
| | (5.286- | (0.033- | (7.194- | (3.697- | (8.536- | | | |
| | 6.556) | 1.337) | 28.723) | 15.626) | 11.516) | | | |

Overall, the antibody was able to neutralize the tested beta viruses SARS-CoV-1, SARS-CoV-2 including variants of concern at varying concentrations (see Table 10), with an overall trend of higher IC₅₀ concentrations for the Omicron variants and Delta. The antibody according to the invention did not neutralize alpha coronaviruses hCoV NL63 and 229E, as expected based on the lack of binding in the MSD binding assessment.

### Example 10. Epitope Mapping

The goal of this study was to precisely determine the amino acid sequence that the antibody binds to in the Spike protein across different alpha and beta coronaviruses. The method includes mapping of linear epitopes using libraries of overlapping synthetic peptides from the spike protein. Briefly, linear epitopes of the sequence of interest were synthesized directly on a solid chip, generating a library of linear mimics, aiding identification of the correct amino acid sequence of the target antibody. The library of peptides consists of overlapping 15-amino acid fragments, with an overlap of 14 amino acids. Binding of the antibody to each of the synthesized peptide chips was tested in an enzyme-linked immunosorbent assay (ELISA) assay via incubation of the peptide arrays with antibody solutions. After washing, peptide arrays were incubated with an antibody peroxidase conjugate and substrate was added, and the reaction was then stopped using hydrogen peroxide. Color development was measured, and intensity was reported.

The antibody bound the peptide array of alpha and beta coronaviruses in a specific epitope of a 10-20 amino acids (see FIG. 9). The method allowed for a high confidence in the epitope definition, with certain variance in the outer areas of the epitope, indicated by asterisk for the respective strain.

1. A method for the treatment of a coronavirus infection in an individual, the method comprising administering to an individual in need thereof an antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628, wherein the antibody is administered to the mucosa.

2. A mucosal composition comprising an antibody comprising a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628.

3. An antibody for use in a method of prevention or treatment of a coronavirus infection in an individual, wherein the antibody comprises a heavy chain variable domain that comprises a heavy chain CDR1 region comprising any one of SEQ ID NO: 004 to 009, 015 to 020, 029 to 034, 039 to 044, 047 to 052, 065 to 070, a heavy chain CDR2 region comprising any one of SEQ ID NO: 082 to 087, 103 to 108, 125 to 130, 136 to 141, 147 to 152, 191 to 196, and a heavy chain CDR3 region comprising any one of SEQ ID NO: 210 to 215, 221 to 226, 245 to 250, 256 to 261, 286 to 291, 297 to 302, a light chain variable domain that comprises a light chain CDR1 region comprising any one of SEQ ID NO: 319 to 324, 330 to 335, 350 to 355, 372 to 377, 383 to 388, 415 to 420, a light chain CDR2 region comprising any one of SEQ ID NO: 432 to 437, 443 to 448, 454 to 459, 465 to 470, 476 to 481, 508 to 513, and a light chain CDR3 region comprising any one of SEQ ID NO: 529 to 534, 540 to 545, 573 to 578, 601 to 606, 612 to 617, 623 to 628, wherein the antibody is administered to mucosa.

4. A method, composition or antibody according to any preceding claim, wherein the antibody comprises a heavy chain variable domain that comprises a heavy chain CDR1 region comprising SEQ ID NO: 029, a heavy chain CDR2 region comprising SEQ ID NO: 136, and a heavy chain CDR3 region comprising SEQ ID NO: 256, a light chain variable domain that comprises a light chain CDR1 region comprising SEQ ID NO: 372, a light chain CDR2 region comprising SEQ ID NO: 432, and a light chain CDR3 region comprising SEQ ID NO: 573.

5. A method, composition or antibody according to any preceding claim, wherein the method for treatment of coronavirus virus infection is a method for prophylactic and/or therapeutic treatment of a coronavirus, preferably wherein the method for treatment is for the prophylactic treatment of a SARS-CoV-2 infection.

6. A method, composition or antibody according to any preceding claim, wherein the antibody comprises a heavy chain variable domain having the sequence of SEQ ID NO: 629 having at most 15, preferably 0, 1, 2, 3, 4, 5, 6, 7, 8, 9 or 10 amino acid insertions, deletions, substitutions which are not in the heavy chain CDRs.

7. A method, composition or antibody according to any preceding claim, wherein the antibody comprises a light chain variable domain having the sequence of SEQ ID NO: 630 having at most 15, preferably 0, 1, 2, 3, 4, 5, 6, 7, 8, 9 or 10 amino acid insertions, deletions, substitutions which are not in the light chain CDRs.

8. A method, composition or antibody according to any preceding claim, wherein the antibody is an IgG antibody, preferably an IgG1 antibody.

9. A method, composition or antibody according to any preceding claim, wherein the antibody is provided to the individual prophylactically.

10. A method, composition or antibody according to any preceding claim, wherein the administration route comprises at least one of oral inhalation, nasal administration, ocular administration and oropharyngeal administration.

11. A method, composition or antibody according to any preceding claim, wherein the antibody is administered at least once or at least twice monthly.

12. A method, composition or antibody according to any preceding claim, wherein the antibody is administered to the individual at a dosage of between 0.01 mg and 20 mg.

13. A composition according to any preceding claim, comprising an antibody in a single dose unit of between 0.01 mg and 20 mg, preferably between 0.1 mg and 15 mg or preferably 0.5 mg and 10 mg, wherein the antibody is as defined in any one of claims 1 to 12.

14. A composition according to any preceding claim, wherein the composition is self-administered.

15. A medicament delivery device comprising a composition according to any preceding claim.


SEQUENCE LISTING (666 sequences)

SEQ ID NO: 1 - protein, 4 aa, Homo sapiens
FTSY

SEQ ID NO: 2 - protein, 5 aa, Homo sapiens
FTSYY

SEQ ID NO: 3 - protein, 6 aa, Homo sapiens
FTSYYM

SEQ ID NO: 4 - protein, 7 aa, Homo sapiens
FTSYYMH

SEQ ID NO: 5 - protein, 8 aa, Homo sapiens
FTSYYMHW

SEQ ID NO: 6 - protein, 9 aa, Homo sapiens
FTSYYMHWV

SEQ ID NO: 7 - protein, 10 aa, Homo sapiens
FTSYYMHWVR

SEQ ID NO: 8 - protein, 11 aa, Homo sapiens
FTSYYMHWVRQ

SEQ ID NO: 9 - protein, 12 aa, Homo sapiens
FTSYYMHWVRQA

SEQ ID NO: 10 - protein, 5 aa, Homo sapiens
GYTFT

SEQ ID NO: 11 - protein, 6 aa, Homo sapiens
GYTFTS

SEQ ID NO: 12 - protein, 7 aa, Homo sapiens
GYTFTSY

SEQ ID NO: 13 - protein, 8 aa, Homo sapiens
GYTFTSYY

SEQ ID NO: 14 - protein, 9 aa, Homo sapiens
GYTFTSYYM

SEQ ID NO: 15 - protein, 10 aa, Homo sapiens
GYTFTSYYMH

SEQ ID NO: 16 - protein, 11 aa, Homo sapiens
GYTFTSYYMHW

SEQ ID NO: 17 - protein, 12 aa, Homo sapiens
GYTFTSYYMHWV

SEQ ID NO: 18 - protein, 13 aa, Homo sapiens
GYTFTSYYMHWVR

SEQ ID NO: 19 - protein, 14 aa, Homo sapiens
GYTFTSYYMHWVRQ

SEQ ID NO: 20 - protein, 15 aa, Homo sapiens
GYTFTSYYMHWVRQA

SEQ ID NO: 21 - protein, 4 aa, Homo sapiens
HWVR

SEQ ID NO: 22 - protein, 5 aa, Homo sapiens
HWVRQ

SEQ ID NO: 23 - protein, 6 aa, Homo sapiens
HWVRQA

SEQ ID NO: 24 - protein, 4 aa, Homo sapiens
MHWV

SEQ ID NO: 25 - protein, 5 aa, Homo sapiens
MHWVR

SEQ ID NO: 26 - protein, 6 aa, Homo sapiens
MHWVRQ

SEQ ID NO: 27 - protein, 7 aa, Homo sapiens
MHWVRQA

SEQ ID NO: 28 - protein, 4 aa, Homo sapiens
SYYM

SEQ ID NO: 29 - protein, 5 aa, Homo sapiens
SYYMH

SEQ ID NO: 30 - protein, 6 aa, Homo sapiens
SYYMHW

SEQ ID NO: 31 - protein, 7 aa, Homo sapiens
SYYMHWV

SEQ ID NO: 32 - protein, 8 aa, Homo sapiens
SYYMHWVR

SEQ ID NO: 33 - protein, 9 aa, Homo sapiens
SYYMHWVRQ

SEQ ID NO: 34 - protein, 10 aa, Homo sapiens
SYYMHWVRQA

SEQ ID NO: 35 - protein, 4 aa, Homo sapiens
TFTS

SEQ ID NO: 36 - protein, 5 aa, Homo sapiens
TFTSY

SEQ ID NO: 37 - protein, 6 aa, Homo sapiens
TFTSYY

SEQ ID NO: 38 - protein, 7 aa, Homo sapiens
TFTSYYM

SEQ ID NO: 39 - protein, 8 aa, Homo sapiens
TFTSYYMH

SEQ ID NO: 40 - protein, 9 aa, Homo sapiens
TFTSYYMHW

SEQ ID NO: 41 - protein, 10 aa, Homo sapiens
TFTSYYMHWV

SEQ ID NO: 42 - protein, 11 aa, Homo sapiens
TFTSYYMHWVR

SEQ ID NO: 43 - protein, 12 aa, Homo sapiens
TFTSYYMHWVRQ

SEQ ID NO: 44 - protein, 13 aa, Homo sapiens
TFTSYYMHWVRQA

SEQ ID NO: 45 - protein, 4 aa, Homo sapiens
TSYY

SEQ ID NO: 46 - protein, 5 aa, Homo sapiens
TSYYM

SEQ ID NO: 47 - protein, 6 aa, Homo sapiens
TSYYMH

SEQ ID NO: 48 - protein, 7 aa, Homo sapiens
TSYYMHW

SEQ ID NO: 49 - protein, 8 aa, Homo sapiens
TSYYMHWV

SEQ ID NO: 50 - protein, 9 aa, Homo sapiens
TSYYMHWVR

SEQ ID NO: 51 - protein, 10 aa, Homo sapiens
TSYYMHWVRQ

SEQ ID NO: 52 - protein, 11 aa, Homo sapiens
TSYYMHWVRQA

SEQ ID NO: 53 - protein, 4 aa, Homo sapiens
WVRQ

SEQ ID NO: 54 - protein, 5 aa, Homo sapiens
WVRQA

SEQ ID NO: 55 - protein, 4 aa, Homo sapiens
YMHW

SEQ ID NO: 56 - protein, 5 aa, Homo sapiens
YMHWV

SEQ ID NO: 57 - protein, 6 aa, Homo sapiens
YMHWVR

SEQ ID NO: 58 - protein, 7 aa, Homo sapiens
YMHWVRQ

SEQ ID NO: 59 - protein, 8 aa, Homo sapiens
YMHWVRQA

SEQ ID NO: 60 - protein, 4 aa, Homo sapiens
YTFT

SEQ ID NO: 61 - protein, 5 aa, Homo sapiens
YTFTS

SEQ ID NO: 62 - protein, 6 aa, Homo sapiens
YTFTSY

SEQ ID NO: 63 - protein, 7 aa, Homo sapiens
YTFTSYY

SEQ ID NO: 64 - protein, 8 aa, Homo sapiens
YTFTSYYM

SEQ ID NO: 65 - protein, 9 aa, Homo sapiens
YTFTSYYMH

SEQ ID NO: 66 - protein, 10 aa, Homo sapiens
YTFTSYYMHW

SEQ ID NO: 67 - protein, 11 aa, Homo sapiens
YTFTSYYMHWV

SEQ ID NO: 68 - protein, 12 aa, Homo sapiens
YTFTSYYMHWVR

SEQ ID NO: 69 - protein, 13 aa, Homo sapiens
YTFTSYYMHWVRQ

SEQ ID NO: 70 - protein, 14 aa, Homo sapiens
YTFTSYYMHWVRQA

SEQ ID NO: 71 - protein, 4 aa, Homo sapiens
YYMH

SEQ ID NO: 72 - protein, 5 aa, Homo sapiens
YYMHW

SEQ ID NO: 73 - protein, 6 aa, Homo sapiens
YYMHWV

SEQ ID NO: 74 - protein, 7 aa, Homo sapiens
YYMHWVR

SEQ ID NO: 75 - protein, 8 aa, Homo sapiens
YYMHWVRQ

SEQ ID NO: 76 - protein, 9 aa, Homo sapiens
YYMHWVRQA

SEQ ID NO: 77 - protein, 16 aa, Homo sapiens
EWMGLITPSGDDTYYA

SEQ ID NO: 78 - protein, 17 aa, Homo sapiens
EWMGLITPSGDDTYYAQ

SEQ ID NO: 79 - protein, 18 aa, Homo sapiens
EWMGLITPSGDDTYYAQR

SEQ ID NO: 80 - protein, 19 aa, Homo sapiens
EWMGLITPSGDDTYYAQRF

SEQ ID NO: 81 - protein, 20 aa, Homo sapiens
EWMGLITPSGDDTYYAQRFQ

SEQ ID NO: 82 - protein, 21 aa, Homo sapiens
EWMGLITPSGDDTYYAQRFQG

SEQ ID NO: 83 - protein, 22 aa, Homo sapiens
EWMGLITPSGDDTYYAQRFQGR

SEQ ID NO: 84 - protein, 23 aa, Homo sapiens
EWMGLITPSGDDTYYAQRFQGRV

SEQ ID NO: 85 - protein, 24 aa, Homo sapiens
EWMGLITPSGDDTYYAQRFQGRVT

SEQ ID NO: 86 - protein, 25 aa, Homo sapiens
EWMGLITPSGDDTYYAQRFQGRVTM

SEQ ID NO: 87 - protein, 26 aa, Homo sapiens
EWMGLITPSGDDTYYAQRFQGRVTMT

SEQ ID NO: 88 - protein, 8 aa, Homo sapiens
GDDTYYAQ

SEQ ID NO: 89 - protein, 9 aa, Homo sapiens
GDDTYYAQR

SEQ ID NO: 90 - protein, 10 aa, Homo sapiens
GDDTYYAQRF

SEQ ID NO: 91 - protein, 11 aa, Homo sapiens
GDDTYYAQRFQ

SEQ ID NO: 92 - protein, 12 aa, Homo sapiens
GDDTYYAQRFQG

SEQ ID NO: 93 - protein, 13 aa, Homo sapiens
GDDTYYAQRFQGR

SEQ ID NO: 94 - protein, 14 aa, Homo sapiens
GDDTYYAQRFQGRV

SEQ ID NO: 95 - protein, 15 aa, Homo sapiens
GDDTYYAQRFQGRVT

SEQ ID NO: 96 - protein, 16 aa, Homo sapiens
GDDTYYAQRFQGRVTM

SEQ ID NO: 97 - protein, 17 aa, Homo sapiens
GDDTYYAQRFQGRVTMT

SEQ ID NO: 98 - protein, 13 aa, Homo sapiens
GLITPSGDDTYYA

SEQ ID NO: 99 - protein, 14 aa, Homo sapiens
GLITPSGDDTYYAQ

SEQ ID NO: 100 - protein, 15 aa, Homo sapiens
GLITPSGDDTYYAQR

SEQ ID NO: 101 - protein, 16 aa, Homo sapiens
GLITPSGDDTYYAQRF

SEQ ID NO: 102 - protein, 17 aa, Homo sapiens
GLITPSGDDTYYAQRFQ

SEQ ID NO: 103 - protein, 18 aa, Homo sapiens
GLITPSGDDTYYAQRFQG

SEQ ID NO: 104 - protein, 19 aa, Homo sapiens
GLITPSGDDTYYAQRFQGR

SEQ ID NO: 105 - protein, 20 aa, Homo sapiens
GLITPSGDDTYYAQRFQGRV

SEQ ID NO: 106 - protein, 21 aa, Homo sapiens
GLITPSGDDTYYAQRFQGRVT

SEQ ID NO: 107 - protein, 22 aa, Homo sapiens
GLITPSGDDTYYAQRFQGRVTM

SEQ ID NO: 108 - protein, 23 aa, Homo sapiens
GLITPSGDDTYYAQRFQGRVTMT

SEQ ID NO: 109 - protein, 11 aa, Homo sapiens
ITPSGDDTYYA

SEQ ID NO: 110 - protein, 12 aa, Homo sapiens
ITPSGDDTYYAQ

SEQ ID NO: 111 - protein, 13 aa, Homo sapiens
ITPSGDDTYYAQR

SEQ ID NO: 112 - protein, 14 aa, Homo sapiens
ITPSGDDTYYAQRF

SEQ ID NO: 113 - protein, 15 aa, Homo sapiens
ITPSGDDTYYAQRFQ

SEQ ID NO: 114 - protein, 16 aa, Homo sapiens
ITPSGDDTYYAQRFQG

SEQ ID NO: 115 - protein, 17 aa, Homo sapiens
ITPSGDDTYYAQRFQGR

SEQ ID NO: 116 - protein, 18 aa, Homo sapiens
ITPSGDDTYYAQRFQGRV

SEQ ID NO: 117 - protein, 19 aa, Homo sapiens
ITPSGDDTYYAQRFQGRVT

SEQ ID NO: 118 - protein, 20 aa, Homo sapiens
ITPSGDDTYYAQRFQGRVTM

SEQ ID NO: 119 - protein, 21 aa, Homo sapiens
ITPSGDDTYYAQRFQGRVTMT

SEQ ID NO: 120 - protein, 17 aa, Homo sapiens
LEWMGLITPSGDDTYYA

SEQ ID NO: 121 - protein, 18 aa, Homo sapiens
LEWMGLITPSGDDTYYAQ

SEQ ID NO: 122 - protein, 19 aa, Homo sapiens
LEWMGLITPSGDDTYYAQR

SEQ ID NO: 123 - protein, 20 aa, Homo sapiens
LEWMGLITPSGDDTYYAQRF

SEQ ID NO: 124 - protein, 21 aa, Homo sapiens
LEWMGLITPSGDDTYYAQRFQ

SEQ ID NO: 125 - protein, 22 aa, Homo sapiens
LEWMGLITPSGDDTYYAQRFQG

SEQ ID NO: 126 - protein, 23 aa, Homo sapiens
LEWMGLITPSGDDTYYAQRFQGR

SEQ ID NO: 127 - protein, 24 aa, Homo sapiens
LEWMGLITPSGDDTYYAQRFQGRV

SEQ ID NO: 128 - protein, 25 aa, Homo sapiens
LEWMGLITPSGDDTYYAQRFQGRVT

SEQ ID NO: 129 - protein, 26 aa, Homo sapiens
LEWMGLITPSGDDTYYAQRFQGRVTM

SEQ ID NO: 130 - protein, 27 aa, Homo sapiens
LEWMGLITPSGDDTYYAQRFQGRVTMT

SEQ ID NO: 131 - protein, 12 aa, Homo sapiens
LITPSGDDTYYA

SEQ ID NO: 132 - protein, 13 aa, Homo sapiens
LITPSGDDTYYAQ

SEQ ID NO: 133 - protein, 14 aa, Homo sapiens
LITPSGDDTYYAQR

SEQ ID NO: 134 - protein, 15 aa, Homo sapiens
LITPSGDDTYYAQRF

SEQ ID NO: 135 - protein, 16 aa, Homo sapiens
LITPSGDDTYYAQRFQ

SEQ ID NO: 136 - protein, 17 aa, Homo sapiens
LITPSGDDTYYAQRFQG

SEQ ID NO: 137 - protein, 18 aa, Homo sapiens
LITPSGDDTYYAQRFQGR

SEQ ID NO: 138 - protein, 19 aa, Homo sapiens
LITPSGDDTYYAQRFQGRV

SEQ ID NO: 139 - protein, 20 aa, Homo sapiens
LITPSGDDTYYAQRFQGRVT

SEQ ID NO: 140 - protein, 21 aa, Homo sapiens
LITPSGDDTYYAQRFQGRVTM

SEQ ID NO: 141 - protein, 22 aa, Homo sapiens
LITPSGDDTYYAQRFQGRVTMT

SEQ ID NO: 142 - protein, 14 aa, Homo sapiens
MGLITPSGDDTYYA

SEQ ID NO: 143 - protein, 15 aa, Homo sapiens
MGLITPSGDDTYYAQ

SEQ ID NO: 144 - protein, 16 aa, Homo sapiens
MGLITPSGDDTYYAQR

SEQ ID NO: 145 - protein, 17 aa, Homo sapiens
MGLITPSGDDTYYAQRF

SEQ ID NO: 146 - protein, 18 aa, Homo sapiens
MGLITPSGDDTYYAQRFQ

SEQ ID NO: 147 - protein, 19 aa, Homo sapiens
MGLITPSGDDTYYAQRFQG

SEQ ID NO: 148 - protein, 20 aa, Homo sapiens
MGLITPSGDDTYYAQRFQGR

SEQ ID NO: 149 - protein, 21 aa, Homo sapiens
MGLITPSGDDTYYAQRFQGRV

SEQ ID NO: 150 - protein, 22 aa, Homo sapiens
MGLITPSGDDTYYAQRFQGRVT

SEQ ID NO: 151 - protein, 23 aa, Homo sapiens
MGLITPSGDDTYYAQRFQGRVTM

SEQ ID NO: 152 - protein, 24 aa, Homo sapiens
MGLITPSGDDTYYAQRFQGRVTMT

SEQ ID NO: 153 - protein, 9 aa, Homo sapiens
PSGDDTYYA

SEQ ID NO: 154 - protein, 10 aa, Homo sapiens
PSGDDTYYAQ

SEQ ID NO: 155 - protein, 11 aa, Homo sapiens
PSGDDTYYAQR

SEQ ID NO: 156 - protein, 12 aa, Homo sapiens
PSGDDTYYAQRF

SEQ ID NO: 157 - protein, 13 aa, Homo sapiens
PSGDDTYYAQRFQ

SEQ ID NO: 158 - protein, 14 aa, Homo sapiens
PSGDDTYYAQRFQG

SEQ ID NO: 159 - protein, 15 aa, Homo sapiens
PSGDDTYYAQRFQGR

SEQ ID NO: 160 - protein, 16 aa, Homo sapiens
PSGDDTYYAQRFQGRV

SEQ ID NO: 161 - protein, 17 aa, Homo sapiens
PSGDDTYYAQRFQGRVT

SEQ ID NO: 162 - protein, 18 aa, Homo sapiens
PSGDDTYYAQRFQGRVTM

SEQ ID NO: 163 - protein, 19 aa, Homo sapiens
PSGDDTYYAQRFQGRVTMT

SEQ ID NO: 164 - protein, 8 aa, Homo sapiens
SGDDTYYA

SEQ ID NO: 165 - protein, 9 aa, Homo sapiens
SGDDTYYAQ

SEQ ID NO: 166 - protein, 10 aa, Homo sapiens
SGDDTYYAQR

SEQ ID NO: 167 - protein, 11 aa, Homo sapiens
SGDDTYYAQRF

SEQ ID NO: 168 - protein, 12 aa, Homo sapiens
SGDDTYYAQRFQ

SEQ ID NO: 169 - protein, 13 aa, Homo sapiens
SGDDTYYAQRFQG

SEQ ID NO: 170 - protein, 14 aa, Homo sapiens
SGDDTYYAQRFQGR

SEQ ID NO: 171 - protein, 15 aa, Homo sapiens
SGDDTYYAQRFQGRV

SEQ ID NO: 172 - protein, 16 aa, Homo sapiens
SGDDTYYAQRFQGRVT

SEQ ID NO: 173 - protein, 17 aa, Homo sapiens
SGDDTYYAQRFQGRVTM

SEQ ID NO: 174 - protein, 18 aa, Homo sapiens
SGDDTYYAQRFQGRVTMT

SEQ ID NO: 175 - protein, 10 aa, Homo sapiens
TPSGDDTYYA

SEQ ID NO: 176 - protein, 11 aa, Homo sapiens
TPSGDDTYYAQ

SEQ ID NO: 177 - protein, 12 aa, Homo sapiens
TPSGDDTYYAQR

SEQ ID NO: 178 - protein, 13 aa, Homo sapiens
TPSGDDTYYAQRF

SEQ ID NO: 179 - protein, 14 aa, Homo sapiens
TPSGDDTYYAQRFQ

SEQ ID NO: 180 - protein, 15 aa, Homo sapiens
TPSGDDTYYAQRFQG

SEQ ID NO: 181 - protein, 16 aa, Homo sapiens
TPSGDDTYYAQRFQGR

SEQ ID NO: 182 - protein, 17 aa, Homo sapiens
TPSGDDTYYAQRFQGRV

SEQ ID NO: 183 - protein, 18 aa, Homo sapiens
TPSGDDTYYAQRFQGRVT

SEQ ID NO: 184 - protein, 19 aa, Homo sapiens
TPSGDDTYYAQRFQGRVTM

SEQ ID NO: 185 - protein, 20 aa, Homo sapiens
TPSGDDTYYAQRFQGRVTMT

SEQ ID NO: 186 - protein, 15 aa, Homo sapiens
WMGLITPSGDDTYYA

SEQ ID NO: 187 - protein, 16 aa, Homo sapiens
WMGLITPSGDDTYYAQ

SEQ ID NO: 188 - protein, 17 aa, Homo sapiens
WMGLITPSGDDTYYAQR

SEQ ID NO: 189 - protein, 18 aa, Homo sapiens
WMGLITPSGDDTYYAQRF

SEQ ID NO: 190 - protein, 19 aa, Homo sapiens
WMGLITPSGDDTYYAQRFQ

SEQ ID NO: 191 - protein, 20 aa, Homo sapiens
WMGLITPSGDDTYYAQRFQG

SEQ ID NO: 192 - protein, 21 aa, Homo sapiens
WMGLITPSGDDTYYAQRFQGR

SEQ ID NO: 193 - protein, 22 aa, Homo sapiens
WMGLITPSGDDTYYAQRFQGRV

SEQ ID NO: 194 - protein, 23 aa, Homo sapiens
WMGLITPSGDDTYYAQRFQGRVT

SEQ ID NO: 195 - protein, 24 aa, Homo sapiens
WMGLITPSGDDTYYAQRFQGRVTM

SEQ ID NO: 196 - protein, 25 aa, Homo sapiens
WMGLITPSGDDTYYAQRFQGRVTMT

SEQ ID NO: 197 - protein, 4 aa, Homo sapiens
AGGF

SEQ ID NO: 198 - protein, 5 aa, Homo sapiens
AGGFD

SEQ ID NO: 199 - protein, 6 aa, Homo sapiens
AGGFDV

SEQ ID NO: 200 - protein, 7 aa, Homo sapiens
AGGFDVW

SEQ ID NO: 201 - protein, 8 aa, Homo sapiens
AGGFDVWG

SEQ ID NO: 202 - protein, 9 aa, Homo sapiens
AGGFDVWGQ

SEQ ID NO: 203 - protein, 10 aa, Homo sapiens
AGGFDVWGQG

SEQ ID NO: 204 - protein, 11 aa, Homo sapiens
AGGFDVWGQGT

SEQ ID NO: 205 - protein, 6 aa, Homo sapiens
AKMSRA

SEQ ID NO: 206 - protein, 7 aa, Homo sapiens
AKMSRAG

SEQ ID NO: 207 - protein, 8 aa, Homo sapiens
AKMSRAGG

SEQ ID NO: 208 - protein, 9 aa, Homo sapiens
AKMSRAGGF

SEQ ID NO: 209 - protein, 10 aa, Homo sapiens
AKMSRAGGFD

SEQ ID NO: 210 - protein, 11 aa, Homo sapiens
AKMSRAGGFDV

SEQ ID NO: 211 - protein, 12 aa, Homo sapiens
AKMSRAGGFDVW

SEQ ID NO: 212 - protein, 13 aa, Homo sapiens
AKMSRAGGFDVWG

SEQ ID NO: 213 - protein, 14 aa, Homo sapiens
AKMSRAGGFDVWGQ

SEQ ID NO: 214 - protein, 15 aa, Homo sapiens
AKMSRAGGFDVWGQG

SEQ ID NO: 215 - protein, 16 aa, Homo sapiens
AKMSRAGGFDVWGQGT

SEQ ID NO: 216 - protein, 7 aa, Homo sapiens
CAKMSRA

SEQ ID NO: 217 - protein, 8 aa, Homo sapiens
CAKMSRAG

SEQ ID NO: 218 - protein, 9 aa, Homo sapiens
CAKMSRAGG

SEQ ID NO: 219 - protein, 10 aa, Homo sapiens
CAKMSRAGGF

SEQ ID NO: 220 - protein, 11 aa, Homo sapiens
CAKMSRAGGFD

SEQ ID NO: 221 - protein, 12 aa, Homo sapiens
CAKMSRAGGFDV

SEQ ID NO: 222 - protein, 13 aa, Homo sapiens
CAKMSRAGGFDVW

SEQ ID NO: 223 - protein, 14 aa, Homo sapiens
CAKMSRAGGFDVWG

SEQ ID NO: 224 - protein, 15 aa, Homo sapiens
CAKMSRAGGFDVWGQ

SEQ ID NO: 225 - protein, 16 aa, Homo sapiens
CAKMSRAGGFDVWGQG

SEQ ID NO: 226 - protein, 17 aa, Homo sapiens
CAKMSRAGGFDVWGQGT

SEQ ID NO: 227 - protein, 4 aa, Homo sapiens
GFDV

SEQ ID NO: 228 - protein, 5 aa, Homo sapiens
GFDVW

SEQ ID NO: 229 - protein, 6 aa, Homo sapiens
GFDVWG

SEQ ID NO: 230 - protein, 7 aa, Homo sapiens
GFDVWGQ

SEQ ID NO: 231 - protein, 8 aa, Homo sapiens
GFDVWGQG

SEQ ID NO: 232 - protein, 9 aa, Homo sapiens
GFDVWGQGT

SEQ ID NO: 233 - protein, 4 aa, Homo sapiens
GGFD

SEQ ID NO: 234 - protein, 5 aa, Homo sapiens
GGFDV

SEQ ID NO: 235 - protein, 6 aa, Homo sapiens
GGFDVW

SEQ ID NO: 236 - protein, 7 aa, Homo sapiens
GGFDVWG

SEQ ID NO: 237 - protein, 8 aa, Homo sapiens
GGFDVWGQ

SEQ ID NO: 238 - protein, 9 aa, Homo sapiens
GGFDVWGQG

SEQ ID NO: 239 - protein, 10 aa, Homo sapiens
GGFDVWGQGT

SEQ ID NO: 240 - protein, 5 aa, Homo sapiens
KMSRA

SEQ ID NO: 241 - protein, 6 aa, Homo sapiens
KMSRAG

SEQ ID NO: 242 - protein, 7 aa, Homo sapiens
KMSRAGG

SEQ ID NO: 243 - protein, 8 aa, Homo sapiens
KMSRAGGF

SEQ ID NO: 244 - protein, 9 aa, Homo sapiens
KMSRAGGFD

SEQ ID NO: 245 - protein, 10 aa, Homo sapiens
KMSRAGGFDV

SEQ ID NO: 246 - protein, 11 aa, Homo sapiens
KMSRAGGFDVW

SEQ ID NO: 247 - protein, 12 aa, Homo sapiens
KMSRAGGFDVWG

SEQ ID NO: 248 - protein, 13 aa, Homo sapiens
KMSRAGGFDVWGQ

SEQ ID NO: 249 - protein, 14 aa, Homo sapiens
KMSRAGGFDVWGQG

SEQ ID NO: 250 - protein, 15 aa, Homo sapiens
KMSRAGGFDVWGQGT

SEQ ID NO: 251 - protein, 4 aa, Homo sapiens
MSRA

SEQ ID NO: 252 - protein, 5 aa, Homo sapiens
MSRAG

SEQ ID NO: 253 - protein, 6 aa, Homo sapiens
MSRAGG

SEQ ID NO: 254 - protein, 7 aa, Homo sapiens
MSRAGGF

SEQ ID NO: 255 - protein, 8 aa, Homo sapiens
MSRAGGFD

SEQ ID NO: 256 - protein, 9 aa, Homo sapiens
MSRAGGFDV

SEQ ID NO: 257 - protein, 10 aa, Homo sapiens
MSRAGGFDVW

SEQ ID NO: 258 - protein, 11 aa, Homo sapiens
MSRAGGFDVWG

SEQ ID NO: 259 - protein, 12 aa, Homo sapiens
MSRAGGFDVWGQ

SEQ ID NO: 260 - protein, 13 aa, Homo sapiens
MSRAGGFDVWGQG

SEQ ID NO: 261 - protein, 14 aa, Homo sapiens
MSRAGGFDVWGQGT

SEQ ID NO: 262 - protein, 4 aa, Homo sapiens
RAGG

SEQ ID NO: 263 - protein, 5 aa, Homo sapiens
RAGGF

SEQ ID NO: 264 - protein, 6 aa, Homo sapiens
RAGGFD

SEQ ID NO: 265 - protein, 7 aa, Homo sapiens
RAGGFDV

SEQ ID NO: 266 - protein, 8 aa, Homo sapiens
RAGGFDVW

SEQ ID NO: 267 - protein, 9 aa, Homo sapiens
RAGGFDVWG

SEQ ID NO: 268 - protein, 10 aa, Homo sapiens
RAGGFDVWGQ

SEQ ID NO: 269 - protein, 11 aa, Homo sapiens
RAGGFDVWGQG

SEQ ID NO: 270 - protein, 12 aa, Homo sapiens
RAGGFDVWGQGT

SEQ ID NO: 271 - protein, 4 aa, Homo sapiens
SRAG

SEQ ID NO: 272 - protein, 5 aa, Homo sapiens
SRAGG

SEQ ID NO: 273 - protein, 6 aa, Homo sapiens
SRAGGF

SEQ ID NO: 274 - protein, 7 aa, Homo sapiens
SRAGGFD

SEQ ID NO: 275 - protein, 8 aa, Homo sapiens
SRAGGFDV

SEQ ID NO: 276 - protein, 9 aa, Homo sapiens
SRAGGFDVW

SEQ ID NO: 277 - protein, 10 aa, Homo sapiens
SRAGGFDVWG

SEQ ID NO: 278 - protein, 11 aa, Homo sapiens
SRAGGFDVWGQ

SEQ ID NO: 279 - protein, 12 aa, Homo sapiens
SRAGGFDVWGQG

SEQ ID NO: 280 - protein, 13 aa, Homo sapiens
SRAGGFDVWGQGT

SEQ ID NO: 281 - protein, 8 aa, Homo sapiens
YCAKMSRA

SEQ ID NO: 282 - protein, 9 aa, Homo sapiens
YCAKMSRAG

SEQ ID NO: 283 - protein, 10 aa, Homo sapiens
YCAKMSRAGG

SEQ ID NO: 284 - protein, 11 aa, Homo sapiens
YCAKMSRAGGF

SEQ ID NO: 285 - protein, 12 aa, Homo sapiens
YCAKMSRAGGFD

SEQ ID NO: 286 - protein, 13 aa, Homo sapiens
YCAKMSRAGGFDV

SEQ ID NO: 287 - protein, 14 aa, Homo sapiens
YCAKMSRAGGFDVW

SEQ ID NO: 288 - protein, 15 aa, Homo sapiens
YCAKMSRAGGFDVWG

SEQ ID NO: 289 - protein, 16 aa, Homo sapiens
YCAKMSRAGGFDVWGQ

SEQ ID NO: 290 - protein, 17 aa, Homo sapiens
YCAKMSRAGGFDVWGQG

SEQ ID NO: 291 - protein, 18 aa, Homo sapiens
YCAKMSRAGGFDVWGQGT

SEQ ID NO: 292 - protein, 9 aa, Homo sapiens
YYCAKMSRA

SEQ ID NO: 293 - protein, 10 aa, Homo sapiens
YYCAKMSRAG

SEQ ID NO: 294 - protein, 11 aa, Homo sapiens
YYCAKMSRAGG

SEQ ID NO: 295 - protein, 12 aa, Homo sapiens
YYCAKMSRAGGF

SEQ ID NO: 296 - protein, 13 aa, Homo sapiens
YYCAKMSRAGGFD

SEQ ID NO: 297 - protein, 14 aa, Homo sapiens
YYCAKMSRAGGFDV

SEQ ID NO: 298 - protein, 15 aa, Homo sapiens
YYCAKMSRAGGFDVW

SEQ ID NO: 299 - protein, 16 aa, Homo sapiens
YYCAKMSRAGGFDVWG

SEQ ID NO: 300 - protein, 17 aa, Homo sapiens
YYCAKMSRAGGFDVWGQ

SEQ ID NO: 301 - protein, 18 aa, Homo sapiens
YYCAKMSRAGGFDVWGQG

SEQ ID NO: 302 - protein, 19 aa, Homo sapiens
YYCAKMSRAGGFDVWGQGT

SEQ ID NO: 303 - protein, 6 aa, Homo sapiens
ASQSIT

SEQ ID NO: 304 - protein, 7 aa, Homo sapiens
ASQSITG

SEQ ID NO: 305 - protein, 8 aa, Homo sapiens
ASQSITGR

SEQ ID NO: 306 - protein, 9 aa, Homo sapiens
ASQSITGRY

SEQ ID NO: 307 - protein, 10 aa, Homo sapiens
ASQSITGRYL

SEQ ID NO: 308 - protein, 11 aa, Homo sapiens
ASQSITGRYLA

SEQ ID NO: 309 - protein, 12 aa, Homo sapiens
ASQSITGRYLAW

SEQ ID NO: 310 - protein, 13 aa, Homo sapiens
ASQSITGRYLAWY

SEQ ID NO: 311 - protein, 14 aa, Homo sapiens
ASQSITGRYLAWYQ

SEQ ID NO: 312 - protein, 15 aa, Homo sapiens
ASQSITGRYLAWYQQ

SEQ ID NO: 313 - protein, 16 aa, Homo sapiens
ASQSITGRYLAWYQQK

SEQ ID NO: 314 - protein, 12 aa, Homo sapiens
ATLSCRASQSIT

SEQ ID NO: 315 - protein, 13 aa, Homo sapiens
ATLSCRASQSITG

SEQ ID NO: 316 - protein, 14 aa, Homo sapiens
ATLSCRASQSITGR

SEQ ID NO: 317 - protein, 15 aa, Homo sapiens
ATLSCRASQSITGRY

SEQ ID NO: 318 - protein, 16 aa, Homo sapiens
ATLSCRASQSITGRYL

SEQ ID NO: 319 - protein, 17 aa, Homo sapiens
ATLSCRASQSITGRYLA

SEQ ID NO: 320 - protein, 18 aa, Homo sapiens
ATLSCRASQSITGRYLAW

SEQ ID NO: 321 - protein, 19 aa, Homo sapiens
ATLSCRASQSITGRYLAWY

SEQ ID NO: 322 - protein, 20 aa, Homo sapiens
ATLSCRASQSITGRYLAWYQ

SEQ ID NO: 323 - protein, 21 aa, Homo sapiens
ATLSCRASQSITGRYLAWYQQ

SEQ ID NO: 324 - protein, 22 aa, Homo sapiens
ATLSCRASQSITGRYLAWYQQK

SEQ ID NO: 325 - protein, 8 aa, Homo sapiens
CRASQSIT

SEQ ID NO: 326 - protein, 9 aa, Homo sapiens
CRASQSITG

SEQ ID NO: 327 - protein, 10 aa, Homo sapiens
CRASQSITGR

SEQ ID NO: 328 - protein, 11 aa, Homo sapiens
CRASQSITGRY

SEQ ID NO: 329 - protein, 12 aa, Homo sapiens
CRASQSITGRYL

SEQ ID NO: 330 - protein, 13 aa, Homo sapiens
CRASQSITGRYLA

SEQ ID NO: 331 - protein, 14 aa, Homo sapiens
CRASQSITGRYLAW

SEQ ID NO: 332 - protein, 15 aa, Homo sapiens
CRASQSITGRYLAWY

SEQ ID NO: 333 - protein, 16 aa, Homo sapiens
CRASQSITGRYLAWYQ

SEQ ID NO: 334 - protein, 17 aa, Homo sapiens
CRASQSITGRYLAWYQQ

SEQ ID NO: 335 - protein, 18 aa, Homo sapiens
CRASQSITGRYLAWYQQK

SEQ ID NO: 336 - protein, 4 aa, Homo sapiens
ITGR

SEQ ID NO: 337 - protein, 5 aa, Homo sapiens
ITGRY

SEQ ID NO: 338 - protein, 6 aa, Homo sapiens
ITGRYL

SEQ ID NO: 339 - protein, 7 aa, Homo sapiens
ITGRYLA

SEQ ID NO: 340 - protein, 8 aa, Homo sapiens
ITGRYLAW

SEQ ID NO: 341 - protein, 9 aa, Homo sapiens
ITGRYLAWY

SEQ ID NO: 342 - protein, 10 aa, Homo sapiens
ITGRYLAWYQ

SEQ ID NO: 343 - protein, 11 aa, Homo sapiens
ITGRYLAWYQQ

SEQ ID NO: 344 - protein, 12 aa, Homo sapiens
ITGRYLAWYQQK

SEQ ID NO: 345 - protein, 10 aa, Homo sapiens
LSCRASQSIT

SEQ ID NO: 346 - protein, 11 aa, Homo sapiens
LSCRASQSITG

SEQ ID NO: 347 - protein, 12 aa, Homo sapiens
LSCRASQSITGR

SEQ ID NO: 348 - protein, 13 aa, Homo sapiens
LSCRASQSITGRY

SEQ ID NO: 349 - protein, 14 aa, Homo sapiens
LSCRASQSITGRYL

SEQ ID NO: 350 - protein, 15 aa, Homo sapiens
LSCRASQSITGRYLA

SEQ ID NO: 351 - protein, 16 aa, Homo sapiens
LSCRASQSITGRYLAW

SEQ ID NO: 352 - protein, 17 aa, Homo sapiens
LSCRASQSITGRYLAWY

SEQ ID NO: 353 - protein, 18 aa, Homo sapiens
LSCRASQSITGRYLAWYQ

SEQ ID NO: 354 - protein, 19 aa, Homo sapiens
LSCRASQSITGRYLAWYQQ

SEQ ID NO: 355 - protein, 20 aa, Homo sapiens
LSCRASQSITGRYLAWYQQK

SEQ ID NO: 356 - protein, 4 aa, Homo sapiens
QSIT

SEQ ID NO: 357 - protein, 5 aa, Homo sapiens
QSITG

SEQ ID NO: 358 - protein, 6 aa, Homo sapiens
QSITGR

SEQ ID NO: 359 - protein, 7 aa, Homo sapiens
QSITGRY

SEQ ID NO: 360 - protein, 8 aa, Homo sapiens
QSITGRYL

SEQ ID NO: 361 - protein, 9 aa, Homo sapiens
QSITGRYLA

SEQ ID NO: 362 - protein, 10 aa, Homo sapiens
QSITGRYLAW

SEQ ID NO: 363 - protein, 11 aa, Homo sapiens
QSITGRYLAWY

SEQ ID NO: 364 - protein, 12 aa, Homo sapiens
QSITGRYLAWYQ

SEQ ID NO: 365 - protein, 13 aa, Homo sapiens
QSITGRYLAWYQQ

SEQ ID NO: 366 - protein, 14 aa, Homo sapiens
QSITGRYLAWYQQK

SEQ ID NO: 367 - protein, 7 aa, Homo sapiens
RASQSIT

SEQ ID NO: 368 - protein, 8 aa, Homo sapiens
RASQSITG

SEQ ID NO: 369 - protein, 9 aa, Homo sapiens
RASQSITGR

SEQ ID NO: 370 - protein, 10 aa, Homo sapiens
RASQSITGRY

SEQ ID NO: 371 - protein, 11 aa, Homo sapiens
RASQSITGRYL

SEQ ID NO: 372 - protein, 12 aa, Homo sapiens
RASQSITGRYLA

SEQ ID NO: 373 - protein, 13 aa, Homo sapiens
RASQSITGRYLAW

SEQ ID NO: 374 - protein, 14 aa, Homo sapiens
RASQSITGRYLAWY

SEQ ID NO: 375 - protein, 15 aa, Homo sapiens
RASQSITGRYLAWYQ

SEQ ID NO: 376 - protein, 16 aa, Homo sapiens
RASQSITGRYLAWYQQ

SEQ ID NO: 377 - protein, 17 aa, Homo sapiens
RASQSITGRYLAWYQQK

SEQ ID NO: 378 - protein, 9 aa, Homo sapiens
SCRASQSIT

SEQ ID NO: 379 - protein, 10 aa, Homo sapiens
SCRASQSITG

SEQ ID NO: 380 - protein, 11 aa, Homo sapiens
SCRASQSITGR

SEQ ID NO: 381 - protein, 12 aa, Homo sapiens
SCRASQSITGRY

SEQ ID NO: 382 - protein, 13 aa, Homo sapiens
SCRASQSITGRYL

SEQ ID NO: 383 - protein, 14 aa, Homo sapiens
SCRASQSITGRYLA

SEQ ID NO: 384 - protein, 15 aa, Homo sapiens
SCRASQSITGRYLAW

SEQ ID NO: 385 - protein, 16 aa, Homo sapiens
SCRASQSITGRYLAWY

SEQ ID NO: 386 - protein, 17 aa, Homo sapiens
SCRASQSITGRYLAWYQ

SEQ ID NO: 387 - protein, 18 aa, Homo sapiens
SCRASQSITGRYLAWYQQ

SEQ ID NO: 388 - protein, 19 aa, Homo sapiens
SCRASQSITGRYLAWYQQK

SEQ ID NO: 389 - protein, 4 aa, Homo sapiens
SITG

SEQ ID NO: 390 - protein, 5 aa, Homo sapiens
SITGR

SEQ ID NO: 391 - protein, 6 aa, Homo sapiens
SITGRY

SEQ ID NO: 392 - protein, 7 aa, Homo sapiens
SITGRYL

SEQ ID NO: 393 - protein, 8 aa, Homo sapiens
SITGRYLA

SEQ ID NO: 394 - protein, 9 aa, Homo sapiens
SITGRYLAW

SEQ ID NO: 395 - protein, 10 aa, Homo sapiens
SITGRYLAWY

SEQ ID NO: 396 - protein, 11 aa, Homo sapiens
SITGRYLAWYQ

SEQ ID NO: 397 - protein, 12 aa, Homo sapiens
SITGRYLAWYQQ

SEQ ID NO: 398 - protein, 13 aa, Homo sapiens
SITGRYLAWYQQK

SEQ ID NO: 399 - protein, 5 aa, Homo sapiens
SQSIT

SEQ ID NO: 400 - protein, 6 aa, Homo sapiens
SQSITG

SEQ ID NO: 401 - protein, 7 aa, Homo sapiens
SQSITGR

SEQ ID NO: 402 - protein, 8 aa, Homo sapiens
SQSITGRY

SEQ ID NO: 403 - protein, 9 aa, Homo sapiens
SQSITGRYL

SEQ ID NO: 404 - protein, 10 aa, Homo sapiens
SQSITGRYLA

SEQ ID NO: 405 - protein, 11 aa, Homo sapiens
SQSITGRYLAW

SEQ ID NO: 406 - protein, 12 aa, Homo sapiens
SQSITGRYLAWY

SEQ ID NO: 407 - protein, 13 aa, Homo sapiens
SQSITGRYLAWYQ

SEQ ID NO: 408 - protein, 14 aa, Homo sapiens
SQSITGRYLAWYQQ

SEQ ID NO: 409 - protein, 15 aa, Homo sapiens
SQSITGRYLAWYQQK

SEQ ID NO: 410 - protein, 11 aa, Homo sapiens
TLSCRASQSIT

SEQ ID NO: 411 - protein, 12 aa, Homo sapiens
TLSCRASQSITG

SEQ ID NO: 412 - protein, 13 aa, Homo sapiens
TLSCRASQSITGR

SEQ ID NO: 413 - protein, 14 aa, Homo sapiens
TLSCRASQSITGRY

SEQ ID NO: 414 - protein, 15 aa, Homo sapiens
TLSCRASQSITGRYL

SEQ ID NO: 415 - protein, 16 aa, Homo sapiens
TLSCRASQSITGRYLA

SEQ ID NO: 416 - protein, 17 aa, Homo sapiens
TLSCRASQSITGRYLAW

SEQ ID NO: 417 - protein, 18 aa, Homo sapiens
TLSCRASQSITGRYLAWY

SEQ ID NO: 418 - protein, 19 aa, Homo sapiens
TLSCRASQSITGRYLAWYQ

SEQ ID NO: 419 - protein, 20 aa, Homo sapiens
TLSCRASQSITGRYLAWYQQ

SEQ ID NO: 420 - protein, 21 aa, Homo sapiens
TLSCRASQSITGRYLAWYQQK

SEQ ID NO: 421 - protein, 4 aa, Homo sapiens
ESSR

SEQ ID NO: 422 - protein, 5 aa, Homo sapiens
ESSRV

SEQ ID NO: 423 - protein, 6 aa, Homo sapiens
ESSRVT

SEQ ID NO: 424 - protein, 7 aa, Homo sapiens
ESSRVTG

SEQ ID NO: 425 - protein, 8 aa, Homo sapiens
ESSRVTGI

SEQ ID NO: 426 - protein, 9 aa, Homo sapiens
ESSRVTGIP

SEQ ID NO: 427 - protein, 10 aa, Homo sapiens
ESSRVTGIPD

SEQ ID NO: 428 - protein, 11 aa, Homo sapiens
ESSRVTGIPDR

SEQ ID NO: 429 - protein, 4 aa, Homo sapiens
GESS

SEQ ID NO: 430 - protein, 5 aa, Homo sapiens
GESSR

SEQ ID NO: 431 - protein, 6 aa, Homo sapiens
GESSRV

SEQ ID NO: 432 - protein, 7 aa, Homo sapiens
GESSRVT

SEQ ID NO: 433 - protein, 8 aa, Homo sapiens
GESSRVTG

SEQ ID NO: 434 - protein, 9 aa, Homo sapiens
GESSRVTGI

SEQ ID NO: 435 - protein, 10 aa, Homo sapiens
GESSRVTGIP

SEQ ID NO: 436 - protein, 11 aa, Homo sapiens
GESSRVTGIPD

SEQ ID NO: 437 - protein, 12 aa, Homo sapiens
GESSRVTGIPDR

SEQ ID NO: 438 - protein, 6 aa, Homo sapiens
LLMYGE

SEQ ID NO: 439 - protein, 7 aa, Homo sapiens
LLMYGES

SEQ ID NO: 440 - protein, 8 aa, Homo sapiens
LLMYGESS

SEQ ID NO: 441 - protein, 9 aa, Homo sapiens
LLMYGESSR

SEQ ID NO: 442 - protein, 10 aa, Homo sapiens
LLMYGESSRV

SEQ ID NO: 443 - protein, 11 aa, Homo sapiens
LLMYGESSRVT

SEQ ID NO: 444 - protein, 12 aa, Homo sapiens
LLMYGESSRVTG

SEQ ID NO: 445 - protein, 13 aa, Homo sapiens
LLMYGESSRVTGI

SEQ ID NO: 446 - protein, 14 aa, Homo sapiens
LLMYGESSRVTGIP

SEQ ID NO: 447 - protein, 15 aa, Homo sapiens
LLMYGESSRVTGIPD

SEQ ID NO: 448 - protein, 16 aa, Homo sapiens
LLMYGESSRVTGIPDR

SEQ ID NO: 449 - protein, 5 aa, Homo sapiens
LMYGE

SEQ ID NO: 450 - protein, 6 aa, Homo sapiens
LMYGES

SEQ ID NO: 451 - protein, 7 aa, Homo sapiens
LMYGESS

SEQ ID NO: 452 - protein, 8 aa, Homo sapiens
LMYGESSR

SEQ ID NO: 453 - protein, 9 aa, Homo sapiens
LMYGESSRV

SEQ ID NO: 454 - protein, 10 aa, Homo sapiens
LMYGESSRVT

SEQ ID NO: 455 - protein, 11 aa, Homo sapiens
LMYGESSRVTG

SEQ ID NO: 456 - protein, 12 aa, Homo sapiens
LMYGESSRVTGI

SEQ ID NO: 457 - protein, 13 aa, Homo sapiens
LMYGESSRVTGIP

SEQ ID NO: 458 - protein, 14 aa, Homo sapiens
LMYGESSRVTGIPD

SEQ ID NO: 459 - protein, 15 aa, Homo sapiens
LMYGESSRVTGIPDR

SEQ ID NO: 460 - protein, 4 aa, Homo sapiens
MYGE

SEQ ID NO: 461 - protein, 5 aa, Homo sapiens
MYGES

SEQ ID NO: 462 - protein, 6 aa, Homo sapiens
MYGESS

SEQ ID NO: 463 - protein, 7 aa, Homo sapiens
MYGESSR

SEQ ID NO: 464 - protein, 8 aa, Homo sapiens
MYGESSRV

SEQ ID NO: 465 - protein, 9 aa, Homo sapiens
MYGESSRVT

SEQ ID NO: 466 - protein, 10 aa, Homo sapiens
MYGESSRVTG

SEQ ID NO: 467 - protein, 11 aa, Homo sapiens
MYGESSRVTGI

SEQ ID NO: 468 - protein, 12 aa, Homo sapiens
MYGESSRVTGIP

SEQ ID NO: 469 - protein, 13 aa, Homo sapiens
MYGESSRVTGIPD

SEQ ID NO: 470 - protein, 14 aa, Homo sapiens
MYGESSRVTGIPDR

SEQ ID NO: 471 - protein, 7 aa, Homo sapiens
RLLMYGE

SEQ ID NO: 472 - protein, 8 aa, Homo sapiens
RLLMYGES

SEQ ID NO: 473 - protein, 9 aa, Homo sapiens
RLLMYGESS

SEQ ID NO: 474 - protein, 10 aa, Homo sapiens
RLLMYGESSR

SEQ ID NO: 475 - protein, 11 aa, Homo sapiens
RLLMYGESSRV

SEQ ID NO: 476 - protein, 12 aa, Homo sapiens
RLLMYGESSRVT

SEQ ID NO: 477 - protein, 13 aa, Homo sapiens
RLLMYGESSRVTG

SEQ ID NO: 478 - protein, 14 aa, Homo sapiens
RLLMYGESSRVTGI

SEQ ID NO: 479 - protein, 15 aa, Homo sapiens
RLLMYGESSRVTGIP

SEQ ID NO: 480 - protein, 16 aa, Homo sapiens
RLLMYGESSRVTGIPD

SEQ ID NO: 481 - protein, 17 aa, Homo sapiens
RLLMYGESSRVTGIPDR

SEQ ID NO: 482 - protein, 4 aa, Homo sapiens
RVTG

SEQ ID NO: 483 - protein, 5 aa, Homo sapiens
RVTGI

SEQ ID NO: 484 - protein, 6 aa, Homo sapiens
RVTGIP

SEQ ID NO: 485 - protein, 7 aa, Homo sapiens
RVTGIPD

SEQ ID NO: 486 - protein, 8 aa, Homo sapiens
RVTGIPDR

SEQ ID NO: 487 - protein, 4 aa, Homo sapiens
SRVT

SEQ ID NO: 488 - protein, 5 aa, Homo sapiens
SRVTG

SEQ ID NO: 489 - protein, 6 aa, Homo sapiens
SRVTGI

SEQ ID NO: 490 - protein, 7 aa, Homo sapiens
SRVTGIP

SEQ ID NO: 491 - protein, 8 aa, Homo sapiens
SRVTGIPD

SEQ ID NO: 492 - protein, 9 aa, Homo sapiens
SRVTGIPDR

SEQ ID NO: 493 - protein, 4 aa, Homo sapiens
SSRV

SEQ ID NO: 494 - protein, 5 aa, Homo sapiens
SSRVT

SEQ ID NO: 495 - protein, 6 aa, Homo sapiens
SSRVTG

SEQ ID NO: 496 - protein, 7 aa, Homo sapiens
SSRVTGI

SEQ ID NO: 497 - protein, 8 aa, Homo sapiens
SSRVTGIP

SEQ ID NO: 498 - protein, 9 aa, Homo sapiens
SSRVTGIPD

SEQ ID NO: 499 - protein, 10 aa, Homo sapiens
SSRVTGIPDR

SEQ ID NO: 500 - protein, 4 aa, Homo sapiens
VTGI

SEQ ID NO: 501 - protein, 5 aa, Homo sapiens
VTGIP

SEQ ID NO: 502 - protein, 6 aa, Homo sapiens
VTGIPD

SEQ ID NO: 503 - protein, 7 aa, Homo sapiens
VTGIPDR

SEQ ID NO: 504 - protein, 4 aa, Homo sapiens
YGES

SEQ ID NO: 505 - protein, 5 aa, Homo sapiens
YGESS

SEQ ID NO: 506 - protein, 6 aa, Homo sapiens
YGESSR

SEQ ID NO: 507 - protein, 7 aa, Homo sapiens
YGESSRV

SEQ ID NO: 508 - protein, 8 aa, Homo sapiens
YGESSRVT

SEQ ID NO: 509 - protein, 9 aa, Homo sapiens
YGESSRVTG

SEQ ID NO: 510 - protein, 10 aa, Homo sapiens
YGESSRVTGI

SEQ ID NO: 511 - protein, 11 aa, Homo sapiens
YGESSRVTGIP

SEQ ID NO: 512 - protein, 12 aa, Homo sapiens
YGESSRVTGIPD

SEQ ID NO: 513 - protein, 13 aa, Homo sapiens
YGESSRVTGIPDR

SEQ ID NO: 514 - protein, 4 aa, Homo sapiens
ASSP

SEQ ID NO: 515 - protein, 5 aa, Homo sapiens
ASSPP

SEQ ID NO: 516 - protein, 6 aa, Homo sapiens
ASSPPT

SEQ ID NO: 517 - protein, 7 aa, Homo sapiens
ASSPPTY

SEQ ID NO: 518 - protein, 8 aa, Homo sapiens
ASSPPTYT

SEQ ID NO: 519 - protein, 9 aa, Homo sapiens
ASSPPTYTF

SEQ ID NO: 520 - protein, 10 aa, Homo sapiens
ASSPPTYTFG

SEQ ID NO: 521 - protein, 11 aa, Homo sapiens
ASSPPTYTFGQ

SEQ ID NO: 522 - protein, 12 aa, Homo sapiens
ASSPPTYTFGQG

SEQ ID NO: 523 - protein, 13 aa, Homo sapiens
ASSPPTYTFGQGT

SEQ ID NO: 524 - protein, 11 aa, Homo sapiens
AVYYCQHFASS

SEQ ID NO: 525 - protein, 12 aa, Homo sapiens
AVYYCQHFASSP

SEQ ID NO: 526 - protein, 13 aa, Homo sapiens
AVYYCQHFASSPP

SEQ ID NO: 527 - protein, 14 aa, Homo sapiens
AVYYCQHFASSPPT

SEQ ID NO: 528 - protein, 15 aa, Homo sapiens
AVYYCQHFASSPPTY

SEQ ID NO: 529 - protein, 16 aa, Homo sapiens
AVYYCQHFASSPPTYT

SEQ ID NO: 530 - protein, 17 aa, Homo sapiens
AVYYCQHFASSPPTYTF

SEQ ID NO: 531 - protein, 18 aa, Homo sapiens
AVYYCQHFASSPPTYTFG

SEQ ID NO: 532 - protein, 19 aa, Homo sapiens
AVYYCQHFASSPPTYTFGQ

SEQ ID NO: 533 - protein, 20 aa, Homo sapiens
AVYYCQHFASSPPTYTFGQG

SEQ ID NO: 534 - protein, 21 aa, Homo sapiens
AVYYCQHFASSPPTYTFGQGT

SEQ ID NO: 535 - protein, 7 aa, Homo sapiens
CQHFASS

SEQ ID NO: 536 - protein, 8 aa, Homo sapiens
CQHFASSP

SEQ ID NO: 537 - protein, 9 aa, Homo sapiens
CQHFASSPP

SEQ ID NO: 538 - protein, 10 aa, Homo sapiens
CQHFASSPPT

SEQ ID NO: 539 - protein, 11 aa, Homo sapiens
CQHFASSPPTY

SEQ ID NO: 540 - protein, 12 aa, Homo sapiens
CQHFASSPPTYT

SEQ ID NO: 541 - protein, 13 aa, Homo sapiens
CQHFASSPPTYTF

SEQ ID NO: 542 - protein, 14 aa, Homo sapiens
CQHFASSPPTYTFG

SEQ ID NO: 543 - protein, 15 aa, Homo sapiens
CQHFASSPPTYTFGQ

SEQ ID NO: 544 - protein, 16 aa, Homo sapiens
CQHFASSPPTYTFGQG

SEQ ID NO: 545 - protein, 17 aa, Homo sapiens
CQHFASSPPTYTFGQGT

SEQ ID NO: 546 - protein, 4 aa, Homo sapiens
FASS

SEQ ID NO: 547 - protein, 5 aa, Homo sapiens
FASSP

SEQ ID NO: 548 - protein, 6 aa, Homo sapiens
FASSPP

SEQ ID NO: 549 - protein, 7 aa, Homo sapiens
FASSPPT

SEQ ID NO: 550 - protein, 8 aa, Homo sapiens
FASSPPTY

SEQ ID NO: 551 - protein, 9 aa, Homo sapiens
FASSPPTYT

SEQ ID NO: 552 - protein, 10 aa, Homo sapiens
FASSPPTYTF

SEQ ID NO: 553 - protein, 11 aa, Homo sapiens
FASSPPTYTFG

SEQ ID NO: 554 - protein, 12 aa, Homo sapiens
FASSPPTYTFGQ

SEQ ID NO: 555 - protein, 13 aa, Homo sapiens
FASSPPTYTFGQG

SEQ ID NO: 556 - protein, 14 aa, Homo sapiens
FASSPPTYTFGQGT

SEQ ID NO: 557 - protein, 5 aa, Homo sapiens
HFASS

SEQ ID NO: 558 - protein, 6 aa, Homo sapiens
HFASSP

SEQ ID NO: 559 - protein, 7 aa, Homo sapiens
HFASSPP

SEQ ID NO: 560 - protein, 8 aa, Homo sapiens
HFASSPPT

SEQ ID NO: 561 - protein, 9 aa, Homo sapiens
HFASSPPTY

SEQ ID NO: 562 - protein, 10 aa, Homo sapiens
HFASSPPTYT

SEQ ID NO: 563 - protein, 11 aa, Homo sapiens
HFASSPPTYTF

SEQ ID NO: 564 - protein, 12 aa, Homo sapiens
HFASSPPTYTFG

SEQ ID NO: 565 - protein, 13 aa, Homo sapiens
HFASSPPTYTFGQ

SEQ ID NO: 566 - protein, 14 aa, Homo sapiens
HFASSPPTYTFGQG

SEQ ID NO: 567 - protein, 15 aa, Homo sapiens
HFASSPPTYTFGQGT

SEQ ID NO: 568 - protein, 6 aa, Homo sapiens
QHFASS

SEQ ID NO: 569 - protein, 7 aa, Homo sapiens
QHFASSP

SEQ ID NO: 570 - protein, 8 aa, Homo sapiens
QHFASSPP

SEQ ID NO: 571 - protein, 9 aa, Homo sapiens
QHFASSPPT

SEQ ID NO: 572 - protein, 10 aa, Homo sapiens
QHFASSPPTY

SEQ ID NO: 573 - protein, 11 aa, Homo sapiens
QHFASSPPTYT

SEQ ID NO: 574 - protein, 12 aa, Homo sapiens
QHFASSPPTYTF

SEQ ID NO: 575 - protein, 13 aa, Homo sapiens
QHFASSPPTYTFG

SEQ ID NO: 576 - protein, 14 aa, Homo sapiens
QHFASSPPTYTFGQ

SEQ ID NO: 577 - protein, 15 aa, Homo sapiens
QHFASSPPTYTFGQG

SEQ ID NO: 578 - protein, 16 aa, Homo sapiens
QHFASSPPTYTFGQGT

SEQ ID NO: 579 - protein, 4 aa, Homo sapiens
SPPT

SEQ ID NO: 580 - protein, 5 aa, Homo sapiens
SPPTY

SEQ ID NO: 581 - protein, 6 aa, Homo sapiens
SPPTYT

SEQ ID NO: 582 - protein, 7 aa, Homo sapiens
SPPTYTF

SEQ ID NO: 583 - protein, 8 aa, Homo sapiens
SPPTYTFG

SEQ ID NO: 584 - protein, 9 aa, Homo sapiens
SPPTYTFGQ

SEQ ID NO: 585 - protein, 10 aa, Homo sapiens
SPPTYTFGQG

SEQ ID NO: 586 - protein, 11 aa, Homo sapiens
SPPTYTFGQGT

SEQ ID NO: 587 - protein, 4 aa, Homo sapiens
SSPP

SEQ ID NO: 588 - protein, 5 aa, Homo sapiens
SSPPT

SEQ ID NO: 589 - protein, 6 aa, Homo sapiens
SSPPTY

SEQ ID NO: 590 - protein, 7 aa, Homo sapiens
SSPPTYT

SEQ ID NO: 591 - protein, 8 aa, Homo sapiens
SSPPTYTF

SEQ ID NO: 592 - protein, 9 aa, Homo sapiens
SSPPTYTFG

SEQ ID NO: 593 - protein, 10 aa, Homo sapiens
SSPPTYTFGQ

SEQ ID NO: 594 - protein, 11 aa, Homo sapiens
SSPPTYTFGQG

SEQ ID NO: 595 - protein, 12 aa, Homo sapiens
SSPPTYTFGQGT

SEQ ID NO: 596 - protein, 10 aa, Homo sapiens
VYYCQHFASS

SEQ ID NO: 597 - protein, 11 aa, Homo sapiens
VYYCQHFASSP

SEQ ID NO: 598 - protein, 12 aa, Homo sapiens
VYYCQHFASSPP

SEQ ID NO: 599 - protein, 13 aa, Homo sapiens
VYYCQHFASSPPT

SEQ ID NO: 600 - protein, 14 aa, Homo sapiens
VYYCQHFASSPPTY

SEQ ID NO: 601 - protein, 15 aa, Homo sapiens
VYYCQHFASSPPTYT

SEQ ID NO: 602 - protein, 16 aa, Homo sapiens
VYYCQHFASSPPTYTF

SEQ ID NO: 603 - protein, 17 aa, Homo sapiens
VYYCQHFASSPPTYTFG

SEQ ID NO: 604 - protein, 18 aa, Homo sapiens
VYYCQHFASSPPTYTFGQ

SEQ ID NO: 605 - protein, 19 aa, Homo sapiens
VYYCQHFASSPPTYTFGQG

SEQ ID NO: 606 - protein, 20 aa, Homo sapiens
VYYCQHFASSPPTYTFGQGT

SEQ ID NO: 607 - protein, 8 aa, Homo sapiens
YCQHFASS

SEQ ID NO: 608 - protein, 9 aa, Homo sapiens
YCQHFASSP

SEQ ID NO: 609 - protein, 10 aa, Homo sapiens
YCQHFASSPP

SEQ ID NO: 610 - protein, 11 aa, Homo sapiens
YCQHFASSPPT

SEQ ID NO: 611 - protein, 12 aa, Homo sapiens
YCQHFASSPPTY

SEQ ID NO: 612 - protein, 13 aa, Homo sapiens
YCQHFASSPPTYT

SEQ ID NO: 613 - protein, 14 aa, Homo sapiens
YCQHFASSPPTYTF

SEQ ID NO: 614 - protein, 15 aa, Homo sapiens
YCQHFASSPPTYTFG

SEQ ID NO: 615 - protein, 16 aa, Homo sapiens
YCQHFASSPPTYTFGQ

SEQ ID NO: 616 - protein, 17 aa, Homo sapiens
YCQHFASSPPTYTFGQG

SEQ ID NO: 617 - protein, 18 aa, Homo sapiens
YCQHFASSPPTYTFGQGT

SEQ ID NO: 618 - protein, 9 aa, Homo sapiens
YYCQHFASS

SEQ ID NO: 619 - protein, 10 aa, Homo sapiens
YYCQHFASSP

SEQ ID NO: 620 - protein, 11 aa, Homo sapiens
YYCQHFASSPP

SEQ ID NO: 621 - protein, 12 aa, Homo sapiens
YYCQHFASSPPT

SEQ ID NO: 622 - protein, 13 aa, Homo sapiens
YYCQHFASSPPTY

SEQ ID NO: 623 - protein, 14 aa, Homo sapiens
YYCQHFASSPPTYT

SEQ ID NO: 624 - protein, 15 aa, Homo sapiens
YYCQHFASSPPTYTF

SEQ ID NO: 625 - protein, 16 aa, Homo sapiens
YYCQHFASSPPTYTFG

SEQ ID NO: 626 - protein, 17 aa, Homo sapiens
YYCQHFASSPPTYTFGQ

SEQ ID NO: 627 - protein, 18 aa, Homo sapiens
YYCQHFASSPPTYTFGQG

SEQ ID NO: 628 - protein, 19 aa, Homo sapiens
YYCQHFASSPPTYTFGQGT

SEQ ID NO: 629 - protein, 118 aa, Homo sapiens
QVQLVQSGAEVKKPGASVKVSCQTSGYTFTSYYMHWVRQAPGQGLEWMGLITPSGDDTYYAQRFQGRVTMTRDTSTSPTYMELSSLTSEDTAVYYCAKMSRAGGFDVWGQGTLVTVSS

SEQ ID NO: 630 - protein, 110 aa, Homo sapiens
EVVLTQSPGTLSLSPGERATLSCRASQSITGRYLAWYQQKPGQAPRLLMYGESSRVTGIPDRFSGGGSGTDFTLTISRLEPEDFAVYYCQHFASSPPTYTFGQGTKLEIR

SEQ ID NO: 631 - protein, 30 aa, Homo sapiens
QVQLVQSGAEVKKPGASVKVSCQTSGYTFT

SEQ ID NO: 632 - protein, 14 aa, Homo sapiens
WVRQAPGQGLEWMG

SEQ ID NO: 633 - protein, 32 aa, Homo sapiens
RVTMTRDTSTSPTYMELSSLTSEDTAVYYCAK

SEQ ID NO: 634 - protein, 11 aa, Homo sapiens
WGQGTLVTVSS

SEQ ID NO: 635 - protein, 23 aa, Homo sapiens
EVVLTQSPGTLSLSPGERATLSC

SEQ ID NO: 636 - protein, 15 aa, Homo sapiens
WYQQKPGQAPRLLMY

SEQ ID NO: 637 - protein, 32 aa, Homo sapiens
GIPDRFSGGGSGTDFTLTISRLEPEDFAVYYC

SEQ ID NO: 638 - protein, 10 aa, Homo sapiens
FGQGTKLEIR

SEQ ID NO: 639 - protein, 5 aa, Homo sapiens
DYRIH

SEQ ID NO: 640 - protein, 17 aa, Homo sapiens
RMNPKSGDTNFAQKFQG

SEQ ID NO: 641 - protein, 15 aa, Homo sapiens
LLIVGGFDPLDDFEV

SEQ ID NO: 642 - protein, 14 aa, Homo sapiens
SGTSSDVGGYNFVS

SEQ ID NO: 643 - protein, 7 aa, Homo sapiens
EVTKRPS

SEQ ID NO: 644 - protein, 10 aa, Homo sapiens
SSYGGTNNLL

SEQ ID NO: 645 - protein, 5 aa, Homo sapiens
GYAMH

SEQ ID NO: 646 - protein, 17 aa, Homo sapiens
VISRDARNKYYADSVKG

SEQ ID NO: 647 - protein, 17 aa, Homo sapiens
LIIPGITEPGSPDALDI

SEQ ID NO: 648 - protein, 11 aa, Homo sapiens
RASQDISKWLA

SEQ ID NO: 649 - protein, 7 aa, Homo sapiens
AASSLQS

SEQ ID NO: 650 - protein, 11 aa, Homo sapiens
QQASSFPWSIT

SEQ ID NO: 651 - protein, 5 aa, Homo sapiens
SHYMH

SEQ ID NO: 652 - protein, 17 aa, Homo sapiens
IINPSGSGTAYGQKFQG

SEQ ID NO: 653 - protein, 8 aa, Homo sapiens
GSGGLFAY

SEQ ID NO: 654 - protein, 12 aa, Homo sapiens
RASQIVRSNYLA

SEQ ID NO: 655 - protein, 7 aa, Homo sapiens
GASSRAT

SEQ ID NO: 656 - protein, 11 aa, Homo sapiens
LQYDSSPPTYI

SEQ ID NO: 657 - protein, 5 aa, Homo sapiens
YFYLH

SEQ ID NO: 658 - protein, 17 aa, Homo sapiens
IINPRGDGTRYAQKFQG

SEQ ID NO: 659 - protein, 9 aa, Homo sapiens
GADHGAFDI

SEQ ID NO: 660 - protein, 12 aa, Homo sapiens
RASQSVRRNYFA

SEQ ID NO: 661 - protein, 7 aa, Homo sapiens
DASTRAT

SEQ ID NO: 662 - protein, 11 aa, Homo sapiens
QQYDSSPPMYI

SEQ ID NO: 663 - protein, 124 aa, Homo sapiens
QMQLMQSGAEVKKPGASVTVSCKASGDTFSDYRIHWVRQAPGQGLEWMGRMNPKSGDTNFAQKFQGRVTMTRDMSINTAYMTLSGLTFDDTALYYCASLLIVGGFDPLDDFEVWGQGTMVTISS

SEQ ID NO: 664 - protein, 110 aa, Homo sapiens
QSALTQPPSASGSPGQSVTISCSGTSSDVGGYNFVSWYQHHPGKAPKILIYEVTKRPSGVPDRFSGSKSGNTASLTVSGLQAEDEADYYCSSYGGTNNLLFGGGTKLTVL

SEQ ID NO: 665 - protein, 126 aa, Homo sapiens [sequence omitted]

SEQ ID NO: 666 - protein, 109 aa, Homo sapiens [sequence omitted]